(19) United States
(12) Patent Application Publication
(10) Pub. No.: US 2026/0096991 A1
(43) Pub. Date: Apr. 9, 2026

(54) BIODEGRADABLE MICROCAPSULES AND METHOD FOR THEIR PREPARATION

(71) Applicant: XAMPLA LIMITED, Cambridge, Cambridgeshire (GB)

(72) Inventors: Lynette Anne Makins HOLLAND, Cambridge, Cambridgeshire (GB); Marc RODRIGUEZ GARCIA, Cambridge, Cambridgeshire (GB); Nigel Patrick SOMERVILLE ROBERTS, Cambridge, Cambridgeshire (GB); Polly Helena Ruth KEEN, Cambridge, Cambridgeshire (GB); James Ward TAYLOR, Cambridge, Cambridgeshire (GB); Juliette Marie Caroline DELARUE, Cambridge, Cambridgeshire (GB); Phoebe Jane WILLIAMS, Cambridge, Cambridgeshire (GB); Huafu WANG, Cambridge, Cambridgeshire (GB); Scott Edward STEVENS, Cambridge, Cambridgeshire (GB)

(21) Appl. No.: 19/115,705

(22) PCT Filed: Sep. 7, 2023
(86) PCT No.: PCT/EP2023/074682

§ 371 Date: Mar. 26, 2025

(30) Foreign Application Priority Data
Sep. 26, 2022 (LU) LU502841

(51) Int. Cl. A61K 9/16; A61K 9/00; A61K 9/50; A61K 31/592

(52) U.S. Cl. CPC A61K 9/1658; A61K 9/0095; A61K 9/1617; A61K 9/5015; A61K 9/5052; A61K 31/592

(57) ABSTRACT

The present invention relates to a method for preparing a microcapsule and to a method for preparing a microcapsule composition. The present invention also relates to the microcapsule and the microcapsule composition per se. The present invention also relates to uses of the microcapsules and to methods involving the microcapsule, including to prepare a formulated product. The present invention also relates to the formulated product per se.

### CROSS-REFERENCE TO RELATED APPLICATION

The present application is a National Stage Application of PCT International Application No.: PCT/EP2023/074682 filed on Sep. 7, 2023, which claims priority to Luxembourg Patent Application LU502841, filed with the Luxembourg Patent Office on Sep. 26, 2022, the disclosure of which is incorporated herein by reference in its entirety.

### FIELD OF THE INVENTION

The present invention relates to a method for preparing a biodegradable microcapsule and to a method for preparing a biodegradable microcapsule composition. The present invention also relates to the biodegradable microcapsule and the biodegradable microcapsule composition per se. The present invention also relates to uses of the biodegradable microcapsules and to methods involving the biodegradable microcapsule, including to prepare a formulated product. The present invention also relates to the formulated product per se.

### BACKGROUND

Encapsulation techniques, which involve embedding an active ingredient(s) in an external matrix, can be used to protect the active ingredient(s) from its external surroundings (e.g. exposure to chemicals, air, light etc.) and also to protect the external surroundings from the active ingredient itself such as when it is hazardous to handle. For example, this allows the active ingredient(s) to be protected when it is added to a product formulation (e.g. a beverage formulation, a fabric conditioner formulation etc.), meaning that the active ingredient(s) in the product formulation has an acceptable shelf life and/or does not activate prematurely. This also means that the external phase of the product formulation is not negatively impacted by the active ingredient(s). The active ingredient(s) is then released when needed (e.g. upon breaking the encapsulate or upon enzymatic degradation of the encapsulate).

Many encapsulation techniques available commonly rely on the use of synthetic polymers to form a protective shell around the active ingredient via a polymerisation process. However, synthetic polymers may not be suitable as shell materials in pharmaceutical and food applications or may have limited permitted exposure levels in cosmetic applications. Furthermore, synthetic polymer shell materials have inherently poor biodegradability and moreover can lead to the formation of microplastics, which are detrimental to the environment.

Drying techniques allow for the large-scale preparation of microcapsules, and spray drying of plant proteins has been attempted to try to achieve production of biodegradable microcapsules or microcapsules for food products. However, in order to achieve robust microcapsules of sufficiently small size via drying techniques, it is necessary that the material has a low viscosity at a high shear rate so that it can be sprayed (e.g. the material needs to be shear thinning to a low enough viscosity for successful spray drying to occur) whilst also having a sufficiently high protein solids content. Conventional methods to prepare a plant protein-based material suitable for spray drying involve hydrolysing the protein to break it down into lower molecular weight fragments (e.g. via treatment with acid or alkali or enzymes). However, the resultant spray-dried microcapsules are not structurally stable in water or under acidic or alkaline conditions, at elevated temperatures and/or under high shear forces, meaning that they are not suitable for incorporation into product formulations, particularly liquid aqueous product formulations, through common manufacturing processes. Furthermore, the processes involved may have a negative impact on the material being encapsulated, as many oils are unstable under alkaline conditions.

The use of hydrogels containing protein in microencapsulation processes is known e.g. from WO2022/221710, which describes a process using a hydrogel for making microcapsules containing an active, typically a flavour. As a fundamental part of this process, the active is first emulsified with an emulsifier (e.g. gum arabic) and a “filler” (e.g. maltodextrin). The emulsion is then mixed with a protein, such as faba bean protein. The mixture is then heated to at least 50° C. over an extended period (at least 30 minutes) to form a hydrogel in-situ which encapsulates the emulsified active. The mixture is then cooled and dried to form particles.

However, the process described in WO2022/221710 is not always well suited for actives that are temperature sensitive or very volatile. This is due to the need to heat the mixture containing the active for extended periods to form the hydrogel. Examples of actives which can be particularly temperature sensitive include volatile fragrances, vitamins and probiotics, which can be prone to thermal degradation. The temperatures experienced during spray drying are typically of less concern due to the very short timescales involved. Microencapsulation processes that avoid the need to subject an active to an extended high temperature step would be advantageous. It is also advantageous to have processes to make microcapsules that can be used in a wide range of products, such as liquid products, especially liquid aqueous products.

The present invention achieves this by forming a protein hydrogel slurry into which the active is dispersed, rather than forming a hydrogel in-situ around droplets of the active. The protein hydrogel slurry is formed by first treating plant protein with solvents and/or pH adjustment to below the isoelectric point etc. followed by shearing. The subsequent incorporation of the active into the protein hydrogel slurry can then be done at room temperature, thus avoiding limitations about active thermal sensitivity.

The hydrogels described and claimed in WO2022/221710 do contain protein, but the use of an emulsifier and “filler” are also required along with the protein. The examples describe the use of gum arabic and glucose at levels almost equal to or greater than the faba bean protein. The resulting hydrogels, and particles formed from them, will have very different properties to those of the present invention, especially regarding their solubility. It is relevant that the examples of WO2022/221710 are for dried products.

Microcapsules that have a significant wall solubility are obviously less suitable for use in liquid products, especially liquid aqueous products. Partial dissolution and/or solubilisation of microcapsule wall materials very typically results in rapid and high active leakage and reduced product stability.

The use of more soluble plant protein materials, such as highly hydrolysed plant proteins, as an encapsulation material for actives in spray drying is well known. The use of partially or fully aqueous soluble materials as an encapsulating material for an immiscible active in a drying process, e.g. spray drying, is typically beneficial to make a high integrity and low porosity microcapsule wall that can retain actives. During the drying process, soluble materials typically dry to make a lower porosity solid, as the dissolved species will progressively fill, and then block, small pores and gaps forming in the microcapsule wall. Use of such soluble materials in an encapsulation and spray drying process can help form high integrity microcapsules that can better retain actives during processing.

Such microcapsules are typically suitable for use in many dry products, such as protein powders and other foodstuffs. Examples are described e.g. in WO2021/165289 and in EP3042571. However, the use of soluble wall materials typically results in microcapsules that are less stable in liquid aqueous compositions with rapid and high loss of active.

The use of a pre-formed protein hydrogel slurry as an encapsulating material for dispersed actives, as described in the present invention, breaks this paradigm. The deformable nature of protein hydrogel fragments allows high integrity microcapsule walls to be formed from a poorly soluble material giving microcapsules that are suitable for use in liquid products. The present invention also allows use of more thermally sensitive materials in these applications.

Accordingly, there exists a need for a method to prepare active ingredient-containing microcapsules of a sufficiently small size for incorporation into a liquid aqueous product formulation, which are both biodegradable and stable in the liquid aqueous product formulation (i.e. the microcapsules retain their structural integrity under either acidic or alkaline conditions such that the active ingredient(s) is protected during manufacture and for the duration of the shelf-life of the product).

### SUMMARY OF THE INVENTION

Viewed from a first aspect, the present invention provides a method for the preparation of a biodegradable microcapsule, the method comprising:
(a) forming a mixture comprising one or more plant-based protein(s) in a solvent system, wherein the solvent system comprises miscible co-solvents; wherein a first co-solvent increases solubility of the plant-based protein(s), and a second co-solvent decreases solubility of the plant-based protein(s); wherein the co-solvents are added to the mixture either in concentrated or in a diluted form; and wherein the pH of the plant-based protein mixture is at least 0.5 pH units below the isoelectric point of the plant-based protein;
(b) subjecting the plant-based protein mixture to a shear treatment to form a plant-based protein hydrogel slurry;
(c) dispersing an active ingredient(s) in said plant-based hydrogel slurry to form a composition; and
(d) drying said composition to form a microcapsule.

Viewed from a further aspect, the present invention provides a biodegradable microcapsule obtained by or obtainable by the method as hereinbefore described.

Viewed from a further aspect, the present invention provides a method for the preparation of a biodegradable microcapsule composition, the method comprising:
(a) preparing a biodegradable microcapsule according to the method as hereinbefore described; and
(b) suspending the biodegradable microcapsule in an external phase.

Viewed from a further aspect, the present invention provides a biodegradable microcapsule composition obtained by or obtainable by the method as hereinbefore described.

Viewed from a further aspect, the present invention provides a spray-dried biodegradable microcapsule comprising an active ingredient(s) and a plant-based protein carrier comprising a plant-based protein(s), wherein the plant-based protein carrier encapsulates the active ingredient(s), and wherein the plant-based protein carrier has a solubility of less than 50% when measured at a protein concentration of 5% w/w in an aqueous solution at pH 7 and 25° C.

Viewed from a further aspect, the present invention provides a composition comprising a biodegradable microcapsule as hereinbefore described and an external phase.

Viewed from a further aspect, the present invention provides a formulated product comprising a biodegradable microcapsule as hereinbefore described.

Viewed from a further aspect, the present invention provides method of making a formulated product, comprising:
(a) preparing a biodegradable microcapsule according to the method as hereinbefore described; and
(b) mixing said biodegradable microcapsule with a product formulation.

Viewed from a further aspect, the present invention provides the use of biodegradable microcapsule as hereinbefore described in a formulated product.

### Definitions

As used herein, the term “microcapsule” refers to any form of microparticle. For example, the term encompasses core-shell microcapsules (i.e. a microcapsule having a central core containing the active ingredient(s), wherein the core is surrounded by a plant-based protein hydrogel in the form of a shell). As will be understood by a skilled person, a core-shell microcapsule may have a multicore morphology (i.e. wherein the core phase is in the form of a plurality of droplets) or a single core morphology (wherein the core phase is in the form of a single droplet). The term also encompasses matrix microcapsules (i.e. a microcapsule comprised of a plant-based protein hydrogel matrix in which the active ingredient(s) has been dispersed throughout). A microcapsule may have a d₅₀ as determined by laser diffraction of between 500 nm and 2 mm.

As used herein, the term “lower shear step” may refer to a process step in which low levels of mechanical energy are applied to a material, preferably by a cutting action, to cause it to break or fragment primarily into large discrete fragments. “Lower shear” does not typically include any milling step that shatters or fragments a material by high-speed impact, for example impacts at a differential velocity of greater than 2 ms⁻¹. Nor does it typically include milling processes based on cavitation. In a particular embodiment, during the lower shear step, a hydrogel is fragmented to give fragments such that at least 80% by weight of the hydrogel fragments have a maximum dimension as determined by optical microscopy, of between 1 mm and 100 mm.

As used herein, the term “higher shear step” may refer to a process step which applies energy to reduce the hydrogel to small fragments, such as to form e.g. a colloidal dispersion. In a particular embodiment, during the higher shear step, a hydrogel is fragmented to give fragments having a d₅₀ particle size as determined by laser diffraction of 0.2 to 50 microns. Laser diffraction can be performed according to the methods defined herein.

For the avoidance of doubt, a higher shear step subjects the hydrogel to higher levels of shear than the lower shear step. In the instance the method involves both a lower shear step and a higher shear step, the higher shear step must happen after the lower shear step (i.e. they are discrete steps occurring in this particular order).

As used herein, the term “sol-gel transition temperature” refers to the temperature at which a plant-based protein transforms from a liquid state into a hydrogel state. Thus, at temperatures above the sol-gel transition temperature, the plant-based protein will be in a liquid state, and at temperatures below the sol-gel transition temperature the plant-based protein will be in a hydrogel state. As used herein, the term “fragrance” (used interchangeably with the term “perfume”) refers to the component of a formulation that is capable of imparting or modifying the odour of a product, such as a fabric conditioner or a hair conditioner or a substrate such as fabric or hair. A fragrance is typically used to impart an overall pleasant odour or odour profile to a product either to provide a pleasurable experience, such as a Fine Fragrance, or to provide sensory cues as to the product's benefit and function, such as a calming effect for a lavender sleep aid, the idea of cleanliness for a laundry product, or to mask an unpleasant odour, such as in an insect repellent product. A “fragrance” may be composed of one or more components that can be a single chemical entity, referred to herein as a “fragrance material” (used interchangeably with the term “perfume material”), or a mixture of different “fragrance materials”. Fragrance materials can be created by either synthetic processes or extracted from nature, particularly plants, to obtain naturally occurring plant essential oils and plant extracts such as orange oil. Fragrance materials created by synthetic processes can be either new-to-the-world chemicals or nature-identical fragrance materials. Synthetic and naturally derived fragrance materials can then be blended into fragrances by skilled perfumers, also called noses, for use in consumer products. Fragrance materials can be obtained from specialist fragrance suppliers, known as fragrance houses, as individual chemicals, natural blends or as proprietary specialty blends where the full composition is not disclosed. The individual fragrance materials which comprise a known natural blend can be found by reference to Journals commonly used by those skilled in the art such as “Perfume and Flavourist” or “Journal of Essential Oil Research”, or listed in reference texts such as the book by S. Arctander, Perfume and Flavor Chemicals, 1969, Montclair, New Jersey, USA and more recently re-published by Allured Publishing Corporation Illinois (1994) and “Perfume and Flavour Materials of Natural Origin”, S. Arctander, Ed., Elizabeth, N.J., 1960. It will be understood that for the purposes of this invention, a “fragrance material” includes a pro-fragrance such as an acetal pro-fragrance, ketal pro-fragrance, ester pro-fragrance, hydrolysable inorganic-organic pro-fragrance, and combinations thereof. The fragrance materials may be released from the pro-fragrances in a number of ways, for example, by hydrolysis, or by a shift in an equilibrium reaction, or by a pH-change, or by enzymatic release, or by UV-radiation.

A fragrance material can be described in terms of its odour strength, detection threshold, odour saturation and its character. In fragrance encapsulates it is preferable to use fragrance materials with a low odour detection threshold and a high strength so as to maximise the noticeability of even small levels of fragrance encapsulated and released.

In order to impart an odour, a fragrance material must be volatile, even if only to a small degree, as it is necessary for the molecule to be airborne to enter the nose, where it gets attached to specific neuroreceptors and elicits a signal within the olfactory system. Fragrance materials can be classified according to their volatility. Preferably fragrance materials are liquid at 20° C. and atmospheric pressure but occasionally they may be solid and can be blended with other liquid fragrance materials or solvents. Typically, the fragrance industry refers to the volatility and substantivity by loosely categorising materials into one of 3 categories: base notes for the least volatile and most substantive, heart notes for those of moderate volatility and substantivity and top notes for the most volatile and least substantive. This is based on the odour perception of the materials and is quite subjective. One way of objectively classifying the volatility of fragrance materials is according to their vapour pressure.

As used herein, the term “vapour pressure” means the partial pressure in air at a defined temperature (e.g., 25° C.) and standard atmospheric pressure (760 mmHg) for a given chemical species. It defines a chemical species' affinity for the gas phase rather than the liquid or solid state. The higher the vapour pressure the greater the proportion of the material that will, at equilibrium, be in a closed headspace. It is also related to the rate of evaporation of a fragrance material which is defined in an open environment where the material is leaving the system. The vapour pressure can be readily determined according to the reference program ACD/Percepta Desktop Software, Version 14.0 (Build: Aug. 26, 2021), Advanced Chemistry Development, Inc (ACD/Labs), Toronto, Canada, www.acdlabs.com.

A physical parameter that is relevant to the encapsulation of a fragrance material is its hydrophobicity, which may be defined in terms of its partition coefficient P. As used herein, the term “partition coefficient” refers to the ratio between the equilibrium concentration of that substance in n-octanol and in water, and is a measure of the differential solubility of said substance between these two solvents. As used herein, the term “log P” refers to the logarithm to the base 10 of the partition coefficient P. The log P can be readily determined according to the reference program ACD/Percepta Desktop Software, Version 14.0 (Build: Aug. 26, 2021), Advanced Chemistry Development, Inc (ACD/Labs), Toronto, Canada, www.acdlabs.com Log P values are predicted from the SMILE string of fragrance materials molecules. Three different types of log P values can be selected from the software. The log P Classic is based on an algorithm which takes into account a database of experimental log P values while using the principal of isolating carbons. The log P GALAS is based on an algorithm taking into account a database of a training set of compounds as well as adjusting the values with data from structurally close compounds. The consensus log P is a model based on the previous two algorithms, which can be expressed as: consensus log P=a×log P Classic+b×log P GALAS, where a and b are coefficients of the model. The latter value, consensus log P, is the log P value indicated herein.

Another aspect that is relevant to the encapsulation of a fragrance material is its Hansen Solubility Parameters (HSPs). The term HSP refers to a solubility parameter approach proposed by Charles Hansen first used to predict polymer solubility in a given solvent as described in, The Three Dimensional Solubility Parameter and Solvent Diffusion Coefficient, by Charles Hansen, Danish Technical Press (Copenhagen, 1967). This approach has since been reapplied to many other molecules. The fragrance material (or flavour material or solvent) and its interactions with its environment are defined by 3 forces: atomic dispersion forces, molecular permanent dipole forces, and molecular hydrogen bonding forces. Materials with similar HSP parameters are more likely to be miscible. These forces can be quantified by 3 values: δD, the Hansen dispersion value which relates to the Van der Waals interactions (intermolecular forces); δP, the Hansen polarity value which relates to the dipole moment (electrical charges); and δH, the Hansen Hydrogen-bonding (“h-bonding”) value. The solubility parameter δ (MPa^{1/2}) is defined as δ²=δ_{D}²+δ_{P}²+δ_{H}²=E/V, where E is the cohesive energy of a solvent and V is the molar volume. The HSP values for a given material can be obtained from the HSPiP (Hansen Solubility Parameters in Practice) software available from www.hansen-solubility.com through two main different ways. Those values can either be retrieved from the Master Dataset, which comprises over 20,000 materials, by searching by name or CAS number; or be predicted by entering the SMILE string of a given molecule in the DIY section of the software, using the Y-MB (Yamamoto-Molecular Breaking) method. Furthermore, the determination of the HSP sphere relative to a given fragrance material is a good way to predict the solubility preferences within a blend of fragrance materials. The radius Ro of the HSP sphere is defined as Ro=Ra/RED, where Ra is the HSP distance between two molecules (1 and 2) expressed by: Ra²=4 (δ_{D1}−δ^{D2})²+(δ^{P1}−δ_{P2})²+(δ_{H1}−δ_{H2})², and RED is the Relative Energy Difference. This RED value can also be extracted from or predicted through the HSPiP software, and a good solvent for a given material should exhibit a RED value lower or equal to 1, whereas a solvent displaying a RED value greater than 1 should be considered as a bad solvent for the given material.

A fragrance material may be selected from an alcohol, an aldehyde, a ketone, an ester, an ether, an acetate, an alkene, a nitrile, a nitrogenous heterocyclic compound, a sulfurous heterocyclic compound, and a Schiff base.

Preferred aldehyde fragrance materials include, without limitation, alpha-amylcinnamaldehyde, anisic aldehyde, decyl aldehyde, lauric aldehyde, methyl n-nonyl acetaldehyde, methyl octyl acetaldehyde, nonylaldehyde, benzenecarboxaldehyde, neral, geranial, 1,1-diethoxy-3,7-dimethylocta-2,6-diene, 4-isopropylbenzaldehyde, 2,4-dimethyl-3-cyclohexene-1-carboxaldehyde, alpha-methyl-p-isopropyldihydrocinnamaldehyde, 3-(3-isopropylphenyl) butanal, alpha-hexylcinnamaldehyde, 7-hydroxy-3,7-dimethyloctan-1-al, 2,4-dimethyl-3-cyclohexene-1-carboxaldehyde, octyl aldehyde, phenylacetaldehyde, 2,4-dimethyl-3-cyclohexene-1-carboxaldehyde, hexanal, 3,7-dimethyloctanal, 6,6-dimethylbicyclo[3.1.1]hept-2-ene-2-butanal, nonanal, octanal, 2-nonenal undecenal, 2-methyl-4-(2,6,6-trimethyl-1-cyclohexenyl-1)-2-butenal, 2,6-dimethyloctanal, 3-(p-isopropylphenyl) propionaldehyde, 3-phenyl-4-pentenal citronellal, o/p-ethyl-alpha, alpha, 9-decenal, dimethyldihydrocinnamaldehyde, p-isobutyl-alphamethylydrocinnamaldehyde, cis-4-decen-1-al, 2,5-dimethyl-2-ethenyl-4-hexenal, trans-2-methyl-2-butenal, 3-methylnonanal, alpha-sinensal, 3-phenylbutanal, 2,2-dimethyl-3-phenylpropionaldehyde, m-tertbutyl-alpha-methyldihydrocinnamic aldehyde, geranyl oxyacetaldehyde, trans-4-decen-1-al, methoxycitronellal, and mixtures thereof.

Preferred ester fragrance materials include, without limitation, allyl cyclohexane-propionate, allyl heptanoate, allyl amyl glycolate, allyl caproate, amyl acetate (n-pentyl acetate), amyl propionate, benzyl acetate, benzyl propionate, benzyl salicylate, cis-3-hexenylacetate, citronellyl acetate, citronellyl propionate, cyclohexyl salicylate, dihydro isojasmonate, dimethyl benzyl carbinyl acetate, ethyl acetate, ethyl acetoacetate, ethyl butyrate, ethyl-2-methyl butryrate, ethyl-2-methyl pentanoate, fenchyl acetate (1,3,3-trimethyl-2-norbornanyl acetate), tricyclodecenyl acetate, tricyclodecenyl propionate, geranyl acetate, cis-3-hexenyl isobutyrate, hexyl acetate, cis-3-hexenyl salicylate, n-hexyl salicylate, isobornyl acetate, linalyl acetate, para-tertiary-butyl cyclohexyl acetate, (−)-L-menthyl acetate, ortho-tertiary-butylcyclohexyl acetate, methyl benzoate, methyl dihydro isojasmonate, alpha-methylbenzyl acetate, methyl salicylate, 2-phenylethyl acetate, prenyl acetate, cedryl acetate, cyclabute, phenethyl phenylacetate, terpinyl formate, citronellyl anthranilate, ethyl tricyclo[5.2.1.0-2,6]decane-2-carboxylate, n-hexyl ethyl acetoacetate, 2-tertbutyl-4-methyl cyclohexyl acetate, formic acid, 3,5,5-trimethylhexyl ester, phenethyl crotonate, cyclogeranyl acetate, geranyl crotonate, ethyl geranate, geranyl isobutyrate, 3,7-dimethyl-ethyl 2-nonynoate-2,6-octadienoic acid methyl ester, citronellyl valerate, 2-hexenylcyclopentanone, cyclohexyl anthranilate, L-citronellyl tiglate, butyl tiglate, pentyl tiglate, geranyl caprylate, 9-decenyl acetate, 2-isopropyl-5-methylhexyl-1 butyrate, n-pentyl benzoate, 2-methylbutyl benzoate (and mixtures thereof with pentyl benzoate), dimethyl benzyl carbinyl propionate, dimethyl benzyl carbinyl acetate, trans-2-hexenyl salicylate, dimethyl benzyl carbinyl isobutyrate, 3,7-dimethyloctyl formate, rhodinyl formate, rhodinyl isovalerate, rhodinyl acetate, rhodinyl butyrate, rhodinyl propionate, cyclohexylethyl acetate, neryl butyrate, tetrahydrogeranyl butyrate, myrcenyl acetate, 2,5-dimethyl-2-ethenylhex-4-enoic acid, methyl ester, 2,4-dimethylcyclohexane-1-methyl acetate, ocimenyl acetate, linalyl isobutyrate, 6-methyl-5-heptenyl-1 acetate, 4-methyl-2-pentyl acetate, n-pentyl 2-methylbutyrate, propyl acetate, isopropenyl acetate, isopropyl acetate, 1-methylcyclohex-3-ene-carboxylic acid, methyl ester, propyl tiglate, propyl/isobutyl cyclopent-3-enyl-1-acetate (alphavinyl), butyl 2-furoate, ethyl 2-pentenoate, (E)-methyl 3-pentenoate, 3-methoxy-3-methylbutyl acetate, n-pentyl crotonate, n-pentyl isobutyrate, propyl formate, furfuryl butyrate, methyl angelate, methyl pivalate, prenyl caproate, furfuryl propionate, diethyl malate, isopropyl 2-methylbutyrate, dimethyl malonate, bornyl formate, styralyl acetate, 1-(2-furyl)-1-propanone, 1-citronellyl acetate, 3,7-dimethyl-1,6-nonadien-3-yl acetate, neryl crotonate, dihydromyrcenyl acetate, tetrahydromyrcenyl acetate, lavandulyl acetate, 4-cyclooctenyl isobutyrate, cyclopentyl isobutyrate, 3-methyl-3-butenyl acetate, allyl acetate, geranyl formate, cis-3-hexenyl caproate, and mixtures thereof.

Preferred alcohol fragrance materials include, without limitation, benzyl alcohol, beta-gamma-hexenol (2-hexen-1-ol), cedrol, citronellol, cinnamic alcohol, p-cresol, cumic alcohol, dihydromyrcenol, 3,7-dimethyl-1-octanol, dimethyl benzyl carbinol, eucalyptol, eugenol, fenchyl alcohol, geraniol, hydratopic alcohol, isononyl alcohol (3,5,5-trimethyl-1-hexanol), linalool, methyl chavicol (estragole), methyl eugenol (eugenyl methyl ether), nerol, 2-octanol, patchouli alcohol, phenyl hexanol (3-methyl-5-phenyl-1-pentanol), phenethyl alcohol, alpha-terpineol, tetrahydrolinalool, tetrahydromyrcenol, 4-methyl-3-decen-5-ol, 1-3,7-dimethyloctane-1-ol, 2-(furfuryl-2)-heptanol, 6,8-dimethyl-2-nonanol, ethyl norbornyl cyclohexanol, beta-methyl cyclohexane ethanol, 3,7-dimethyl-(2),6-octen (adien)-1-ol, trans-2-undecen-1-ol, 2-ethyl-2-prenyl-3-hexenol, isobutyl benzyl carbinol, dimethyl benzyl carbinol, ocimenol, 3,7-dimethyl-1,6-nonadien-3-ol (cis & trans), tetrahydromyrcenol, alpha-terpineol, 9-decenol-1,2-(2-hexenyl)-cyclopentanol, 2,6-dimethyl-2-heptanol, 3-methyl-1-octen-3-ol, 2,6-dimethyl-5-hepten-2-ol, 3,7,9-trimethyl-1,6-decadien-3-ol, 3,7-dimethyl-6-nonen-1-ol, 3,7-dimethyl-1-octyn-3-ol, 2,6-dimethyl-1,5,7-octatrienol-3, dihydromyrcenol, 2,6-trimethyl-5,9-undecadienol, 2,5-dimethyl-2-propylhex-4-enol-1, (Z)-3-hexenol, o,m,p-methyl-phenylethanol, 2-methyl-5-phenyl-1-pentanol, 3-methylphenethyl alcohol, para-methyl dimethyl benzyl carbinol, methyl benzyl carbinol, p-methylphenylethanol, 3,7-dimethyl-2-octen-1-ol, 2-methyl-6-methylene-7-octen-4-ol, and mixtures thereof.

Preferred ketone fragrance materials include, without limitation, oxacycloheptadec-10-en-2-one, benzylacetone, benzophenone, L-carvone, cis-jasmone, 4-(2,6,6-trimethyl-3-cyclohexen-1-yl)-but-3-en-4-one, ethyl amyl ketone, alpha-ionone, ionone beta, ethanone, octahydro-2,3,8,8-tetramethyl-2-acetonaphthalene, alpha-irone, 1-(5,5-dimethyl-1-cyclo-hexen-1-yl)-4-penten-1-one, 3-nonanone, ethyl hexyl ketone, menthone, 4-methyl-acetophenone, gamma-methyl ionone, methyl pentyl ketone, methyl heptenone (6-methyl-5-hepten-2-one), methyl heptyl ketone, methyl hexyl ketone, delta muscenone, 2-octanone, 2-pentyl-3-methyl-2-cyclopenten-1-one, 2-heptylcyclopentanone, alpha-methylionone, 3-methyl-2-(trans-2-pentenyl)-cyclopentenone, octenyl cyclopentanone, n-amylcyclopentenone, 6-hydroxy-3,7-dimethyloctanoic acid lactone, 2-hydroxy-2-cyclohexen-1-one, 3-methyl-4-phenyl-3-buten-2-one, 2-pentyl-2,5,5-trimethylcyclopentanone, 2-cyclopentylcyclopentanol-1,5-methylhexan-2-one, gamma-dodecalactone, delta-dodecalactone, gamma-nonalactone, delta-nonalactone, gamma-octalactone, delta-undecalactone, gamma-undecalactone, alpha damascone, beta damascone, gamma damascone, delta damascone and mixtures thereof.

Preferred ether fragrance materials include, without limitation, diphenyl oxide, p-cresyl methyl ether, 4,6,6,7,8,8-hexamethyl-1,3,4,6,7,8-hexahydro-cyclopenta (G)-2-benzopyran, beta-naphthyl methyl ether, methyl isobutenyl tetrahydropyran, 5-acetyl-1,1,2,3,3,6-hexamethylindan (phantolide), 7-acetyl-1,1,3,4,4,6-hexamethyltetralin (tonalid), 2-phenylethyl-3-methylbut-2-enyl ether, ethyl geranyl ether, phenylethyl isopropyl ether, and mixtures thereof.

Preferred alkene fragrance materials include, without limitation, allo-ocimene, camphene, beta-caryophyllene, cadinene, diphenylmethane, d-limonene, lymolene, beta-myrcene, para-cymene, 2-alpha-pinene, beta-pinene, alpha-terpinene, gamma-terpinene, terpineolene, 7-methyl-3-methylene-1,6-octadiene, and mixtures thereof.

Preferred nitrile fragrance materials include, without limitation, 3,7-dimethyl-6-octenenitrile, 3,7-dimethyl-2 (3), 6-nonadienenitrile, (2E,6Z)-2,6-nonadienenitrile, n-dodecane nitrile, and mixtures thereof.

Preferred Schiff base fragrance materials include, without limitation, citronellyl nitrile, nonanal/methyl anthranilate, N-octylidene-anthranilic acid methyl ester, hydroxycitronellal/methyl anthranilate, cyclamen aldehyde/methyl anthranilate, methoxyphenylpropanal/methyl anthranilate, ethyl p-aminobenzoate/hydroxycitronellal, citral/methyl anthranilate, 2,4-dimethylcyclohex-3-enecarbaldehyde methyl anthranilate, hydroxycitronellal-indole, and mixtures thereof.

As used herein, the term “flavour” refers to the component of a formulation that is capable of imparting or modifying the taste and smell of a product, such as a toothpaste or foodstuff. A flavour is typically used to impart an overall pleasant taste and smell, or a taste and smell profile, to a product either to simply provide a pleasurable experience, such as in a foodstuff, or to mask an unpleasant taste or smell, such as in a medicine. A flavour or flavour material can be described in terms of its aroma strength, detection threshold and quality. A “flavour” may be composed of one or more components that can be a single chemical entity, referred to herein as a “flavour material”, or a mixture of different “flavour materials”. Flavour materials can be created by either synthetic processes or extracted from nature, particularly from plants, to create naturally occurring plant and animal oils and exudate, such as vanilla. Synthetic and naturally derived flavour materials can then be blended into flavours by skilled flavourists for use in consumer products. Flavour materials can be obtained from specialist flavour suppliers, known as flavour houses, as individual chemicals, natural blends or as proprietary specialty blends where the full composition is not disclosed. The individual flavour materials which comprise a known natural blend can be found by reference to Journals commonly used by those skilled in the art such as “Perfume and Flavourist” or “Journal of Essential Oil Research”, or listed in reference texts such as the book by S. Arctander, Perfume and Flavor Chemicals, 1969, Montclair, New Jersey, USA and more recently re-published by Allured Publishing Corporation Illinois (1994); “Perfume and Flavour Materials of Natural Origin”, S. Arctander, Ed., Elizabeth, N.J., 1960; and “Flavourings”, E. Ziegler and H. Ziegler (ed.), Wiley-VCH Weinheim, 1998. It will be understood that flavours can be volatile or have volatile components which are detected by the nose in the same way as fragrances. As such, flavour materials can also be classified according to their physical characteristics, such as volatility and hydrophobicity, using the methods described above for fragrance materials. Flavour materials can also be described according to their Hansen Solubility Parameters using the methods described above for fragrance materials.

Sources of flavour materials include essential oils, concretes, absolutes, resins, resinoids, balsams, and tinctures. Preferred flavour materials include anise oil, ethyl-2-methyl butyrate, vanillin, cis-3-heptenol, cis-3-hexenol, trans-2-heptenal, butyl valerate, 2,3-diethyl pyrazine, methylcyclo-pentenolone, benzaldehyde, valerian oil, 3,4-dimeth-oxyphenol, amyl acetate, amyl cinnamate, γ-butyryl lactone, trimethyl pyrazine, phenyl acetic acid, isovaleraldehyde, ethyl maltol, ethyl vanillin, ethyl valerate, ethyl butyrate, cocoa extract, coffee extract, peppermint oil, spearmint oil, clove oil, anethol, cardamom oil, wintergreen oil, cinnamic aldehyde, ethyl-2-methyl valerate, g-hexenyl lactone, 2,4-decadienal, 2,4-heptadienal, methyl thiazole alcohol (4-methyl-5-b-hydroxyethyl thiazole), 2-methyl butanethiol, 4-mercapto-2-butanone, 3-mercapto-2-pentanone, 1-mercapto-2-propane, benzaldehyde, furfural, furfuryl alcohol, 2-mercapto propionic acid, alkyl pyrazine, methyl pyrazine, 2-ethyl-3-methyl pyrazine, tetramethyl pyrazine, polysulfides, dipropyl disulphide, methyl benzyl disulphide, alkyl thiophene, 2,3-dimethyl thiophene, 5-methyl furfural, acetyl furan, 2,4-decadienal, guiacol, phenyl acetaldehyde, b-decalactone, d-limonene, acetoin, amyl acetate, maltol, ethyl butyrate, levulinic acid, piperonal, ethyl acetate, n-octanal, n-pentanal, n-hexanal, diacetyl, monosodium glutamate, monopotassium glutamate, sulfur-containing amino acids, e.g., cysteine, 2-methylfuran-3-thiol, 2-methyldihydrofuran-3-thiol, 2,5-dimethylfuran-3-thiol, tetramethyl pyrazine, propylpropenyl disulphide, propylpropenyl trisulfide, diallyl disulphide, diallyl trisulfide, dipropenyl disulphide, dipropenyl trisulfide, 4-methyl-2-[(methylthio)-ethyl]-1,3-dithiolane, 4,5-dimethyl-2-(methylthiomethyl)-1,3-dithiolane, and 4-methyl-2-(methylthiomethyl)-1,3-dithiolane, hop oils, and citrus oils such as lemon, orange, lime, and grapefruit.

### DETAILED DESCRIPTION OF THE INVENTION

The inventors of the present invention have uncovered a method to prepare a high solids content protein colloidal dispersion of controlled particle size and low viscosity comprising an active ingredient(s), which allows for the formation of stable microcapsules upon drying. The resultant microcapsules are stable in liquid product formulations, and also under the conditions necessary to prepare such a liquid product formulation (e.g. pasteurisation conditions in the case of a beverage formulation).

The present invention is directed to a method for the preparation of a biodegradable microcapsule, the method comprising:
(a) forming a mixture comprising one or more plant-based protein(s) in a solvent system, wherein the solvent system comprises miscible co-solvents; wherein a first co-solvent increases solubility of the plant-based protein(s), and a second co-solvent decreases solubility of the plant-based protein(s); wherein the co-solvents are added to the mixture either in concentrated or in a diluted form; and wherein the pH of the plant-based protein mixture is at least 0.5 pH units below the isoelectric point of the plant-based protein;
(b) subjecting the plant-based protein mixture to a shear treatment to form a plant-based protein hydrogel slurry;
(c) dispersing an active ingredient(s) in said plant-based hydrogel slurry to form a composition; and
(d) drying said composition to form a microcapsule.

Any suitable plant-based proteins may be used in the present invention. In preferred methods of the present invention, the plant-based protein(s) is obtained from fava bean, mung bean, pea, rice, potato, rapeseed, lentil, chickpea, sunflower seed, pumpkin seed, flax, chia, canola, lupine, alfalfa, moringa, wheat, corn zein or sorghum; preferably the plant protein(s) is selected from pea protein, potato protein, rapeseed protein, lentil protein, chickpea protein, fava bean protein, mung bean protein, sunflower seed protein, pumpkin seed protein, flax protein, chia protein, canola protein, lupine protein, alfalfa protein, moringa protein and/or rice protein. More preferably, the plant-based protein is pea protein and/or potato protein. Such proteins are considered to be low allergenicity proteins.

Suitable plant-based proteins further include:

Brassicas: including Brassica balearica: Mallorca cabbage, Brassica carinata: Abyssinian mustard or Abyssinian cabbage, Brassica elongata: elongated mustard, Brassica fruticulosa: Mediterranean cabbage, Brassica hilarionis: St Hilarion cabbage, Brassica juncea: Indian mustard, brown and leaf mustards, Sarepta mustard, Brassica napus: rapeseed, canola, rutabaga, Brassica narinosa: broadbeaked mustard, Brassica nigra: black mustard, Brassica oleracea: kale, cabbage, collard greens, broccoli, cauliflower, kai-lan, Brussels sprouts, kohlrabi, Brassica perviridis: tender green, mustard spinach, Brassica rapa (syn. B. campestris): Chinese cabbage, turnip, rapini, komatsuna, Brassica rupestris: brown mustard, Brassica tournefortii: Asian mustard

Solanaceae: including tomatoes, potatoes, eggplant, bell and chili peppers;
cereals: including maize, rice, wheat, barley, sorghum, millet, oats, rye, triticale, fonio pseudocereals: including amaranth (love-lies-bleeding, red amaranth, prince-of-Wales-feather), breadnut, buckwheat, chia, cockscomb (also called quail grass or soko), pitseed Goosefoot, qañiwa, quinoa and, wattleseed (also called acacia seed);

Legume: including Acacia alata (Winged Wattle), Acacia decipiens, Acacia saligna (commonly known by various names including coojong, golden wreath wattle, orange wattle, blue-leafed wattle), Arachis hypogaea (peanut), Astragalus galegiformis, Cytisus laburnum (the common laburnum, golden chain or golden rain), Cytisus supinus, Dolichios lablab (common names include hyacinth bean, lablab-bean bonavist bean/pea, dolichos bean, seim bean, lablab bean, Egyptian kidney bean, Indian bean, bataw and Australian pea.), Ervum lens (Lentil), Genista tinctorial (common names include dyer's whin, waxen woad and waxen wood), Glycine max (Soybean), Lathyrus clymenum (peavines or vetchlings), Lathyrus odoratus (peavines or vetchlings), Lathyrus staivus (peavines or vetchlings), Lathyrus Silvetris (peavines or vetchlings), Lotus tetragonolobus (asparagus-pea or winged pea), Lupinus albus (Lupin), Lupinus angustifolius (lupin), Lupinus luteus (Lupin), Lupinus polyphyllus (Lupin), Medicago sativa (Alfalfa), Phaseolus aureus (Mung bean), Phaseolus coccineus (Runner bean), Phaseolus nanus (Green bean/French bean), Phaseolus vulgaris (Green bean/French bean), Pisum sativum (pea), Trifolium hybridum (Clover), Trifolium pretense (Red clover), Vicia faba (Broad bean), Vicia sativa (Vetch), Vigna unguiculate (cowpea)

Non-Legumes: including: Acanshosicyos horrida (Acanshosicyos horrida), Aesculus hyppocastanum (Conker tree/Horsechestnut), Anacardium occidentale (Cashew tree), Balanites aegyptica, Bertholletia excels (Brazil nut), Beta vulgaris (Sugar beet), Brassica napus (Rapeseed), Brassica juncea (Brown mustard), Brassica nigra (Black mustard), Brassica hirta (Eurasian mustard), Cannabis sativa (marijuana), Citrullus vulgaris (Sort of watermelon), Citrus aurantiaca (Citrus), Cucurbita maxima (squash), Fagopyrum esculentum (knotweed), Gossypium barbadense (Extra-long staple cotton), Heianthus annuus (sunflower), Nicotiana sp. (Tobacco plant), Prunus avium (cherry), Prunus cerasus (Sour cherry), Prunus domestica (plum), Prunus amygdalus (almond), Rricinus communis (Caster bean/caster oil plant), Sasamum indicum (Sesame), Sinapis alba (White mustard), Terlfalrea pedata (Oyster nut).

For the avoidance of doubt, the plant-based microcapsules of the present invention do not encompass plants in their natural state, e.g. naturally formed plant cells, organelles or vesicles are not plant-based microcapsules of the present invention.

In step (a), the first co-solvent increases solubility of the plant-based protein(s). The first co-solvent may be considered a solubilising co-solvent. There may be one or more solubilising co-solvent(s) and the solubilising co-solvent(s) may fully or partially solubilise the plant-based protein(s). The co-solvents may be added in step (a) in a highly concentrated form or in a diluted form.

Examples of solubilising co-solvents are organic acids. An organic acid is an organic compound with acidic properties. Preferably, the organic acids are sourced from natural plant-based or bio-based feed-stocks.

In preferred methods of the present invention, the first co-solvent is an organic acid. Preferably, the organic acid is acetic acid, lactic acid, formic acid, propionic acid, an α-hydroxy acid and/or a β-hydroxy acid. Preferred α-hydroxy acids include glycolic acid, lactic acid, acetic acid, malic acid, citric acid, maleic acid, gluconic and/or tartaric acid, preferably lactic acid or acetic acid. Preferred β-hydroxy acid may include β-hydroxypropionic acid, β-hydroxybutyric acid, β-hydroxy β-methylbutyric acid, 2-hydroxybenzoic acid and carnitine. In particularly preferred methods of the present invention, the organic acid is acetic acid and/or lactic acid.

Using an organic acid enables solubilisation of the plant protein and also allows for mild hydrolysis of the protein. For example, without wishing to be bound by theory, the solubility of plant-based proteins in organic acid is possible due to: i) the protonation of proteins and ii) the presence of an anion solvation layer which contributes to a reduction of hydrophobic interactions. Once initially dissolved in organic acid, the protonation of plant-based proteins can help to stabilise them in its non-solvent, for example water.

In step (a), the second co-solvent has decreased solubility of the plant based protein(s), as compared to the first co-solvent. The second co-solvent may be considered a de-solubilising co-solvent. There may be one or more de-solubilising co-solvent(s).

In preferred methods of the present invention, the second co-solvent is selected from water, ethanol, and/or ethyl acetate, more preferably water and/or ethanol, even more preferably water.

In preferred methods of the present invention, the solvent system comprises a co-solvent ratio of first co-solvent to second co-solvent of about 10-90% v/v, preferably about 20-90% v/v, preferably about 20-80% v/v, preferably about 20-60% v/v, about 25-55% v/v, about 30-50% v/v, about 20%, about 30%, about 40% about 50% or about 60% v/v, most preferably about 30-50% v/v.

In accordance with the present invention it may be preferred that the first co-solvent is present in the mixture of step (a) at a concentration based on weight-% of equal or greater than the concentration of the protein.

In preferred methods of the present invention the pH of the plant-based protein mixture in step (a) is at least 0.5 pH units, more preferably at least 1.0 pH units, below the isoelectric point of the plant-based protein.

In preferred methods of the present invention, the concentration of the plant-based protein(s) in the solvent system is 25-200 mg/ml, more preferably 50-150 mg/ml.

The ratio of organic acid may vary depending on protein concentration, e.g. using a higher organic acid ratio with increasing protein concentration.

In preferred methods of the present invention, the degree of protein hydrolysis (i.e. the percentage of cleaved peptide bonds in a protein hydrolysate) is controlled to modify the properties of the resultant hydrogel. For example, increasing the acid concentration present during formation will increase the degree of protein hydrolysis. Higher degree of protein hydrolysis leads to the formation of less rigid hydrogels.

In preferred methods of the present invention, the degree of protein hydrolysis is 0.1 to 10%, preferably 0.1 to 5%, even more preferably 0.1 to 2.5%.

In order to form the mixture comprising one or more plant-based protein(s), it may be necessary to apply physical stimulus to the protein/solvent system mixture to enable interaction between the solvent and the protein. Suitable physical stimulus includes heating, ultrasonication, agitation, high-shear mixing, high-shear homogenisation or other physical techniques. A preferred technique is heating, optionally with subsequent ultrasonication.

Preferably, the protein/solvent system mixture is subjected to a physical stimulus which is heating, wherein the mixture is heated to about or above 70° C. More preferably, the protein/solvent system mixture is heated to about or above 75° C., about or above 80° C., about or above 85° C. or about 90° C. Even more preferably, the protein/solvent system mixture is heated to 85° C.

Preferably, the protein/solvent system mixture is subjected to a physical stimulus which is heating for a period of about 5 minutes, 10 minutes, 15 minutes, 20 minutes, 25 minutes, 30 minutes, or greater than 30 minutes. The heated protein/solvent system mixture is optionally subjected to subsequent ultrasonication.

In preferred methods of the present invention, the protein mixture is heated to a first temperature above the sol-gel transition temperature of the one or more plant-based protein(s) mixture, then reduced to a second temperature below the sol-gel transition temperature of the one or more plant-based protein(s) mixture to form a hydrogel, preferably between step (a) and step (b) or during step (b).

The protein mixture is heated such that the liquid mixture is held above the sol-gel transition for the protein(s). By modifying the solvent system (for example through selection of the choice of organic acid, the ratio of organic acid to further solvent or through further means) it is possible to modify the sol-gel transition temperature for the protein(s). Through appropriate selection of conditions, it is possible to carefully control the sol-gel transition of the protein thereby controlling the formation of the hydrogel.

Preferably, the protein mixture is heated to about or above 70° C. More preferably, the protein is heated to about or above 75° C., about or above 80° C., about or above 85° C. or about 90° C. Even more preferably, the protein is heated to 85° C.

The protein mixture may be held at elevated temperature for a time period of about 5 minutes, 10 minutes, 15 minutes, 20 minutes, 25 minutes, 30 minutes, 45 minutes or 1 hour. A preferred time period is at least 30 minutes to enable the proteins to fully solubilise. It is possible to hold the protein mixture at an elevated temperature for a longer period of time.

Having heated the protein mixture to above the sol-gel transition temperature, the temperature of the protein mixture can be reduced to a second temperature below the sol-gel transition temperature to facilitate formation of the hydrogel. The second temperature may be room temperature. The second temperature may be in the range 5 to 25° C., preferably 10 to 20° C. The protein mixture may be held at the reduced temperature for long periods of time, e.g. days, weeks prior to performing the shear treatment in step (b). The protein mixture may be held at the reduced temperature for a time period of about 5 minutes, 10 minutes, 15 minutes, 20 minutes, 25 minutes or about 30 minutes. A particular reduced time period is about 5 minutes.

The particular temperatures will depend on the properties of the protein source, the solvent conditions used and therefore the sol-gel transition temperature. Alternatively, the elevated and reduced temperatures may be relatively fixed (for example about 85° C. then about room temperature) and the co-solvent mixture conditions are adjusted to ensure a suitable sol-gel transition temperature for the selected plant-based protein.

Without wishing to be bound by theory, it is believed that when the plant protein is added to the solvent system the plant protein forms a dispersion of insoluble colloidal protein aggregates. Aggregate size may be measured by Dynamic Light Scattering (DLS). Suitable apparatus to measure aggregate size is a Zetasizer Nano S (Malvern).

It is believed that upon heating the protein mixture in the presence of a co-solvent system to above the sol-gel transition temperature, the plant proteins partially unfold, resulting in the exposure of hydrophobic amino acids initially buried within the protein native structure. Once partially unfolded, the co-solvents are able to interact with the unfolded protein molecules. For example, an organic acid has greater access to protonate amino acid residues, as well as enabling the formation of anion salt bridges that stabilise hydrophobic interactions. Also, upon heating at elevated temperatures, protein-protein non-covalent intermolecular contacts are disrupted.

Further, it is believed that the application of mechanical agitation, for example ultrasonication, disrupts large colloidal protein aggregates into smaller ones, as well as disrupting protein intermolecular interactions.

Further, it is believed that when there is cooling of the protein mixture to below the sol-gel transition temperature, protein-protein non-covalent intermolecular contacts are enabled, thus promoting the self-assembly of plant protein molecules into a hydrogel of inter-connected protein aggregates.

It is believed that the method of the present invention allows the plant proteins to aggregate into supramolecular structures held by intermolecular hydrogen bonding interactions, and in particular between the β-strands.

The methods of the present invention enable materials to be formed in which there are high levels of β-sheet intermolecular interactions. Thus, in methods of the present invention, the plant-based protein(s) have a protein secondary structure with at least 40% intermolecular β-sheet, at least 50% intermolecular β-sheet, at least 60% intermolecular β-sheet, at least 70% intermolecular β-sheet, at least 80% intermolecular β-sheet, or at least 90% intermolecular β-sheet, wherein the % intermolecular β-sheet content is measured by FTIR (Fourier transform infrared spectroscopy).

In preferred methods of the present invention, the plant-based protein hydrogel is subjected to a solvent reduction step, preferably a solubilising solvent reduction step, preferably between step (a) and step (b) or during step (b).

By solubilising solvent, we mean a solvent or mixture of solvents in which the plant-based protein hydrogel dissolves. Examples include organic acids: such as acetic acid, lactic acid, formic acid, propionic acid, an α-hydroxy acid and/or a B-hydroxy acid. The a-hydroxy acid may preferably be selected from glycolic acid, acetic acid, lactic acid, malic acid, citric acid, maleic acid, gluconic acid and/or tartaric acid. The B-hydroxy acid may preferably be selected from B-hydroxypropionic acid, β-hydroxybutyric acid, β-hydroxy β-methylbutyric acid, 2-hydroxybenzoic acid and carnitine.

In preferred methods of the present invention, said solvent reduction step involves:
(i) contacting the plant-based protein hydrogel with a non-solubilising solvent;
(ii) separating the plant-based hydrogel from the non-solubilising solvent to give a washed plant-based protein hydrogel; and
(iii) optionally repeating steps (i) and (ii).

Step (i) involves contacting the plant-based protein hydrogel with a non-solubilising solvent. By non-solubilising solvent, we mean a solvent or mixture of solvents in which the plant-based protein hydrogel does not dissolve. Examples include water or a mixture of water and ethanol.

In preferred methods of the present invention, said shear treatment comprises one step (i.e. a single shear step). The single shear step may be a higher shear step. Preferably, said single shear step involves fragmenting the plant-based protein hydrogel into fragments.

In preferred methods of the present invention, said fragments produced in the single shear step have a d₅₀ as determined by laser diffraction of 0.2 to 50 microns, preferably 1 to 40 microns, preferably 2 to 30 microns.

In preferred methods of the present invention, said single shear step involves ultrasonication (e.g. using equipment such as a Bandelin HD4200 or a Hielscher UIP1000hdT), high-shear mechanical stirring (e.g. using equipment such as a Silverson rotor-stator high-shear mixer), high-pressure homogenisation, or cavitation, preferably ultrasonication.

In preferred methods of the present invention, the single shear step is conducted at a temperature that is below the sol-gel transition temperature of the plant-based protein mixture. In preferred methods of the present invention, said first shear step is conducted for a duration of at least 5 minutes, more preferably at least 1 minute.

In preferred methods of the present invention, said shear treatment comprises two steps. Preferably, said shear treatment comprises a first shear step followed by a second shear step. The first shear step may be a lower shear step and the second shear step may be a higher shear step.

In preferred methods of the present invention, said first shear step involves fragmenting the plant-based protein hydrogel into fragments. Preferably, at least 50 wt % of said fragments produced in said first shear step have a particle dimension in the range 1 mm to 100 mm, preferably 1 mm to 50 mm, preferably 5 mm to 30 mm, more preferably 10 mm to 30 mm. More preferably, at least 80 wt % of said fragments produced in said first shear step have a particle dimension in the range 1 mm to 100 mm, preferably 1 mm to 50 mm, preferably 5 mm to 30 mm, more preferably 10 mm to 30 mm. This can be measured by optical microscopy, or visually.

In preferred methods of the present invention, the first shear step is conducted at a temperature that is below the sol-gel transition temperature of the plant-based protein mixture.

In preferred methods of the present invention, said first shear step involves mechanical cutting. By mechanical cutting, we mean cutting using a knife edge (e.g. a knife, an extruder blade etc.)

In alternative preferred methods of the present invention, said first shear step involves extrusion. For example, the plant-based protein mixture formed in step (a) can be extruded into a non-solubilising solvent (e.g. water) to form the plant-based protein hydrogel in large discrete fragments, e.g. the large discrete fragments may take the form of extrudates having a thread or string form. In this way, the fragments can be directly subjected to a solvent reduction step, as described in more detail below. A first shear step of this nature is more amenable to large scale processing. In this case, the first shear step may reduce the at least one dimension of the large fragment to between 1 mm and 100 mm, for example a diameter of the extrudate. Preferably, at least 50 wt % of said fragments produced in said first shear step have at least one internal dimension in the range 1 mm to 100 mm, preferably 1 mm to 50 mm, preferably 5 mm to 30 mm, more preferably 10 mm to 30 mm. More preferably, at least 80 wt % of said fragments produced in said first shear step have at least one internal dimension in the range 1 mm to 100 mm, preferably 1 mm to 50 mm, preferably 5 mm to 30 mm, more preferably 10 mm to 30 mm. This can be measured by optical microscopy, or visually.

In preferred methods of the present invention, said second shear step involves further fragmenting the plant-based protein hydrogel. Preferably, said fragments produced in the second shear step have a d₅₀ as determined by laser diffraction of 0.2 to 50 microns, preferably 1 to 40 microns, preferably 2 to 30 microns.

In preferred methods of the present invention, the particle size distribution of the hydrogel fragments in the plant-based protein hydrogel slurry can be adjusted by varying the nature and the intensity of the second shear step. In another preferred method, the particle size distribution of the hydrogel fragments in the plant-based protein hydrogel slurry can be adjusted by blending or combining two or more different hydrogel slurries that have been subjected to different second shear steps and having different particle size distributions.

In preferred methods of the present invention, said second shear step is conducted at a temperature that is below the sol-gel transition temperature of the plant-based protein(s).

In preferred methods of the present invention, said second shear step is conducted at a temperature that is below the protein denaturation temperature of the plant-based protein(s).

In preferred methods of the present invention, said second shear step is conducted for a duration of at least 5 minutes, more preferably at least 1 minute.

In preferred methods of the present invention, said second shear step involves ultrasonication (e.g. using equipment such as a Bandelin HD4200 or a Hielscher UIP1000hdT), high-shear mechanical stirring (e.g. using equipment such as a Silverson rotor-stator high-shear mixer), high pressure homogenisation, or cavitation, preferably ultrasonication.

In preferred methods of the present invention, step (b) further comprises subjecting the plant-based protein hydrogel slurry to a solvent reduction step, preferably a solubilising solvent reduction step, between said first shear step and said second shear step.

In preferred methods of the present invention, said solvent reduction step comprises the steps of:
(i) contacting the fragments of the plant-based hydrogel slurry with a non-solubilising solvent;
(ii) separating the fragments of the plant-based hydrogel slurry from the non-solubilising solvent to give a washed plant-based protein hydrogel; and
(iii) optionally repeating steps (i) and (ii).

Solubilising solvents are as defined above.

Step (i) involves contacting the fragments of the plant-based protein hydrogel slurry with a non-solubilising solvent. Non-solubilising solvents are as defined above.

In preferred methods of the present invention, step (ii) involves mesh filtration or centrifugation. More preferably, step (ii) involves mesh filtration using multiple meshes of decreasing size.

As would be understood by a skilled person, if the fragments produced in the first shear step are too small the solvent reduction step can prove difficult as the fragments can end up blocking the meshes or the collected yield is low. However, if the fragments produced in the first shear step are too large, the solvent reduction step can take excessive amounts of time due to the slow mass transport of solvent from the core of the fragments.

Without wishing to be bound by theory, it is thought that due to the porous nature of the hydrogel, the solvent reduction step can remove some or all of the solvent (e.g. organic acid) from the hydrogel via a solvent exchange.

The strength of a protein hydrogel can be altered by varying the concentration of protein and organic acid, amongst other variables.

It is useful for the strength of the hydrogel used to form the hydrogel slurries to be within certain limits. This can be measured by oscillatory rheometry. A suitable measure of hydrogel strength is the storage modulus, G′, of the hydrogel. Suitable test conditions are 1% strain at an oscillatory frequency of 1 Hz at 20° C. Suitable equipment is an Anton Paar MCR 92 Rheometer with a 50 mm diameter, 1-degree angle cone and plate measurement geometry.

Thus, in preferred methods of the present invention, prior to washing said plant-based protein hydrogel has a storage modulus (G′) at 10 rad/s of greater than 1000 Pa, preferably greater than 2000 Pa, more preferably greater than 5000 Pa, even more preferably greater than 6000 Pa, most preferably greater than 8000 Pa. As would be understood by a skilled person, 2× rad/s is equivalent to 1 Hz.

In preferred methods of the present invention, prior to washing said plant-based protein hydrogel has a storage modulus (G′) at 10 rad/s of less than 20,000 Pa, preferably less than 15,000 Pa, more preferably less than 10,000 Pa.

In preferred methods of the present invention, prior to washing said plant-based protein hydrogel has a storage modulus (G′) at 10 rad/s of between about 1000 to 20,000 Pa, preferably between about 2000 to 15,000 Pa, more preferably between about 2000 to 10,000 Pa.

Further, in preferred methods of the present invention, the washed plant-based protein hydrogel has a storage modulus (G′) at 10 rad/s of greater than 200 Pa, preferably greater than 250 Pa, more preferably greater than 300 Pa, even more preferably greater than 350 Pa, more preferably greater than 400 Pa, more preferably greater than 450 Pa.

In preferred methods of the present invention, the washed plant-based protein hydrogel has a storage modulus (G′) at 10 rad/s of less than 5000 Pa, preferably less than 2500 Pa, preferably less than 1000 Pa, preferably less than 950 Pa, more preferably less than 900 Pa, more preferably less than 850 Pa, more preferably less than 800 Pa, more preferably less than 750 Pa.

In preferred methods of the present invention, the washed plant-based protein hydrogel has a storage modulus (G′) at 10 rad/s of between about 200 to 5000 Pa, between about 200 to 2500 Pa, between about 200 to 1000 Pa, between about 250 to 950 Pa, between about 300 to 900 Pa, between about 350 to 850 Pa, between about 400 to 800 Pa, or between about 450 to 750 Pa.

Preferred methods of the present invention further comprise adding an additional ingredient to the plant-based protein hydrogel slurry in step (b) or between steps (b) and (c), or to the composition of step (c). Preferably, said additional ingredient is selected from plasticisers, surfactants, rheology modifiers, opacifiers, preservatives, pigments, carbohydrates, gums, polymers, and nanoparticles, or mixtures thereof.

In preferred methods of said present invention, said additional ingredient is a plasticiser. Preferably, said plasticiser is selected from glycerol, ethylene glycol, triethylene glycol, tetraethylene glycol, polyethylene glycol, propylene glycol, sorbitol, mannitol, xylitol, fatty acids, glucose, mannose, fructose, sucrose, lactic acid, citric acid, glycolic acid, malic acid, gluconic acid, tartaric acid, ethanolamine, urea, triethanolamine, vegetable oils, lecithin, waxes and amino acids.

Preferred methods of the present invention further comprise a step of altering the pH of the plant-based protein hydrogel slurry such that it is different to the isoelectric point of the plant-based protein by more than 1 pH unit.

Preferably, said step of altering the pH of the plant-based protein hydrogel slurry is carried out after step (b). Alternatively, said step of altering the pH of the plant-based protein hydrogel slurry is carried out sequentially with step (b).

During adjustment of the pH of the plant-based protein hydrogel slurry, it is possible for the slurry to pass through the isoelectric point of the protein. Due to the lack of charge repulsion at the isoelectric point, the dispersed protein fragments in the plant-based protein hydrogel slurry can quickly coagulate. To avoid this, pH modification materials can be used to rapidly change the pH and therefore minimise the time that the slurry is at the isoelectric point. The isoelectric point of a particular plant-based protein can be determined using the method described in Helmick et al., Food Biophysics (2021) 16:474-483, or may be available in the literature, e. g. in Guldekin et al., Food Hydrocolloids (2023), 145:109029.

Thus, in preferred methods of the present invention, said step of altering the pH of the plant-based protein hydrogel slurry involves adding a pH-modification material to the plant-based protein hydrogel slurry. Preferably, said pH-modification material is a solution comprising monovalent metal ions, divalent metal ions or ammonium ions, preferably an aqueous alkaline solution comprising monovalent metal ions, divalent metal ions or ammonium ions. More preferably, said pH-modification material is an aqueous hydroxide solution, preferably sodium hydroxide, potassium hydroxide, or ammonium hydroxide.

In preferred methods of the present invention, the pH of the plant-based protein hydrogel slurry after said step of altering the pH of the plant-based protein hydrogel slurry is below the isoelectric point of the plant-based protein by at least 1 pH unit.

In preferred methods of the present invention, the pH of the plant-based protein hydrogel slurry after said step of altering the pH of the plant-based protein hydrogel slurry is above the isoelectric point of the plant-based protein by at least 1 pH unit.

As would be understood by a skilled person, the addition of an additional ingredient(s) to the plant-based protein hydrogel slurry in step (b) or between step (b) and (c) or to the composition of step (c) may impact the pH of the slurry. As such, the step of altering the pH of the plant-based protein hydrogel slurry preferably takes place after the addition of any additional ingredient(s).

In preferred methods of the present invention, the composition formed in step (c) is a shear thinning composition.

In preferred methods of the present invention, the composition formed in step (c) has a viscosity in the range 1 to 10000 cP at 20° C. and 50 s⁻¹, preferably in the range 10 to 7500 cP at 20° C. and 50 s⁻¹, preferably in the range 15 to 5000 cP at 20° C. and 50 s⁻¹.

In preferred methods of the present invention, the composition formed in step (c) has a protein solids content in the range 1 wt % to 25 wt % based upon the total weight of the composition, preferably 2 wt % to 20 wt %, more preferably 4 wt % to 15 wt %, even more preferably 5 wt % to 12 wt %. The term “protein solids” refers to the dried dispersed 30 hydrogel solids that originate from the plant protein added in step (a). The plant protein is typically a plant protein isolate that is mainly composed of material chemically identifiable as protein but also includes a low level of other insoluble plant materials such as fibres. The % of protein solids is measured according to the method described herein.

In preferred methods of the present invention, the active ingredient(s) is selected from a vitamin, a mineral, a flavour material, a fragrance material, a pro-flavour, a pro-fragrance, a flavour enhancer, a malodour counteractant, a nutraceutical, a live organism (e.g. a probiotic), a pharmaceutical, an anti-microbial agent, an anti-viral agent, an anti-inflammatory agent, a pesticide, a herbicide, a fertiliser, a fungicide, an insecticide, an animal repellent, an anti-acne agent, a skin lightening agent, an emollient, a skin moisturizing agent, a wrinkle control agent, a fabric softener active, a surface cleaning active, a skin conditioning agent, a hair conditioning agent, a dye, a pigment, and an adhesive, or combinations thereof.

In particularly preferred methods of the present invention, the active ingredient(s) is at least one fragrance material or flavour material. Preferably, the at least one fragrance material or flavour material is selected from an alcohol, an aldehyde, a ketone, an ester, an ether, an acetate, an alkene, a nitrile, a nitrogenous heterocyclic compound, a sulfurous heterocyclic compound, and a Schiff base.

The fragrance materials and flavour materials employed in the present invention may be of natural origin (i.e. they are extracted from a natural source and are not synthetically modified in any way). Preferred fragrance materials or flavour materials of natural origin include nutmeg extract, cardamon extract, ginger extract, cinnamon extract, patchouli oil, geranium oil, orange oil, mandarin oil, orange flower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomile oil, clove oil, sage oil, neroli oil, labdanum oil, eucalyptus oil, verbena oil, mimosa extract, narcissus extract, jasmine extract, olibanum extract, rose extract, vanillin, coffee extract, hop oil or combinations thereof. Preferably, the fragrance materials or flavour materials of natural origin are plant-derived. The fragrance materials or flavour materials of natural origin may be used alone, or in combination, or in combination with synthetic fragrance materials.

In preferred methods of the present invention, the at least one fragrance material or flavour material has a vapour pressure greater than or equal to 0.00001 Torr at 25° C.

In preferred methods of the present invention, the at least one fragrance material or flavour material has a log P greater than or equal to 3.0, preferably greater than or equal to 3.5, more preferably greater than or equal to 4.0.

In preferred methods of the present invention, the at least one fragrance material or flavour material has at least two Hansen solubility parameters selected from: an atomic dispersion force (δD) from 14 to 20, a dipole moment (δP) of less than 8, and a hydrogen bonding (δH) from 2.5 to 11.

In preferred methods of the present invention, the at least one fragrance material or flavour material is part of a fragrance or flavour.

Preferably, the fragrance or flavour contains at least 20 wt % of fragrance material(s) or flavour material(s) with a log P greater than or equal to 3.0 based upon the total weight of the fragrance or flavour, more preferably greater than or equal to 3.5, more preferably greater than or equal to 4.0.

Preferably, the fragrance or flavour contains at least 40 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 50 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 60 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 10 wt % of fragrance material(s) or flavour material(s) of natural origin based upon the total weight of the fragrance or flavour, preferably at least 30 wt %, more preferably at least 50 wt %, more preferably at least 70 wt %.

Preferably, the fragrance or flavour contains at least 10 wt % of fragrance material(s) or flavour material(s) which have a biodegradation percentage based upon O₂ consumption as measured according to ISO-14851 version 2019 after 28 days of 60 to 100% based upon the ratio of the Biological Oxygen Demand (BOD) to the Theoretical Oxygen Demand, more preferably 65%, even more preferably 70 to 100%, even more preferably 75 to 100%, even more preferably 80 to 100%, even more preferably 85 to 100%, most preferably 90 to 100%. Due to their low aqueous solubility, fragrance or 30 flavours can be added to the biodegradation test on an inert support according to the method in ISO 10634 version 2018: Water quality—Preparation and treatment of poorly water-soluble organic compounds for the subsequent evaluation of their biodegradability in an aqueous medium. In addition, the fragrance or flavour can be checked for any inhibitory effect on the microorganisms as specified in the method.

Preferably, the fragrance or flavour contains at least 80 wt %, preferably at least 90 wt %, more preferably at least 95 wt % of fragrance material(s) or flavour material(s) having at least two Hansen solubility parameters selected from: an atomic dispersion force (δD) from 14 to 20, a dipole moment (δP) of less than 8, and a hydrogen bonding (δH) from 2.5 to 11, based upon the total weight of the fragrance or flavour.

Preferably, the fragrance or flavour contains only low levels of materials with an alcohol functionality (e.g. a primary alcohol functionality). In preferred methods of the present invention, the fragrance or flavour comprises less than 40% wt alcohol-containing material based upon the total weight of the fragrance or flavour, more preferably less than 20% wt. In particularly preferred methods of the present invention, the fragrance or flavour does not comprise an alcohol-containing material. Without wishing to be bound by theory, it is thought that alcohols, and particularly primary alcohols having a straight chain alkyl group, can easily diffuse through the shell of a microcapsule due to their structure, meaning that they can be difficult materials to encapsulate.

Preferably, the fragrance or flavour material is of high odour impact. This is advantageous as it ensures that even low levels of fragrance are perceived when released from the microcapsules.

In alternative particularly preferred methods of the present invention, the active ingredient(s) is a vitamin or a mineral. Preferably, said active ingredient(s) is a vitamin or a mineral selected from Vitamin A, Vitamin B1, Vitamin B2, Vitamin B3, Vitamin B5, Vitamin B6, Vitamin B7, Vitamin B9, Vitamin B12, Vitamin C, Vitamin D, Vitamin E, Vitamin K, magnesium, sodium, potassium, zinc, iron, calcium, iodine, flaxseed oil, omega 3 fatty acid, folic acid, thiamin, riboflavin, niacin and phosphorous, or mixtures thereof. More preferably, said active ingredient is Vitamin D.

In alternative particularly preferred methods of the present invention, an additive can be included in the microcapsule core to act as an active protectant to reduce any potential degradation of the active ingredient(s) during processing and storage. Preferably, the core additive is an antioxidant or free radical scavenger such as Vitamin E and curcumin. Preferably, the core additive is a pigment such as carotenoids, preferably beta-carotene. Preferably, the core additive is a natural product with a mixture of antioxidants, such as polyphenols, and other compounds such as olive oil and turmeric.

The vitamins and minerals employed in the present invention may be sourced from a vitamin-containing and/or mineral-containing material. An example of such a material is seaweed.

In alternatively preferred methods of the present invention the active ingredient is an agrochemical, such as a pesticide, a herbicide, a fertiliser, a fungicide, an insecticide, an animal repellent, or combinations thereof.

Preferably, the agrochemicals are a natural alternative to synthetic materials and are based on plant extracts and/or plant essential oils (EOs) or components of essential oils, such as thymol. Preferably, the agrochemical is a biopesticide. Preferably, the agrochemicals are suitable for use in formulations for plant care and production that can be certified as organic by organisations, such as the USDA (US Department of Agriculture) or Ecocert in Europe.

In preferred methods of the present invention, the active ingredient(s) is part of a composition comprising the active ingredient(s) and an active carrier phase. Preferably, the active carrier phase is a solvent, a fat or a wax.

In preferred methods of the present invention, the active carrier phase is a solvent. Preferably, the solvent is a solvent with low volatility (e.g. having a vapour pressure of less than 0.1 Torr at 25° C., preferably less than 0.01 Torr at 25° C., preferably less than 0.001 Torr at 25° C.). Preferably, the solvent has low or no odour.

Preferably, the solvent has at least two Hansen solubility parameters selected from: an atomic dispersion force (δD) of less than 20, a dipole moment (δP) of less than 8, and a hydrogen bonding (δH) of less than 11. More preferably, the solvent has at least two Hansen solubility parameters selected from: an atomic dispersion force (δD) of less than 20, a dipole moment (δP) of less than 4, and a hydrogen bonding (δH) of less than 5.

Preferably, the solvent has a density of greater than 1.07 g/cm³. Solvents having this property are advantageously able to prevent creaming of the encapsulates (e.g. in a final product formulation).

Preferably, the solvent contains only low levels of materials with an alcohol functionality (e.g. a primary alcohol functionality). In preferred methods of the present invention, the solvent comprises less than 40% wt alcohol-containing material based upon the total weight of the solvent, more preferably less than 20% wt. In particularly preferred methods of the present invention, the solvent does not comprise an alcohol-containing material.

In preferred methods of the present invention, the active carrier phase is a solvent selected from a carboxylic acid ester, a fatty acid ester, a phthalate ester, a triol, a diol, a rosin resin, an isoparaffin, a terpene, and a vegetable oil, or combinations thereof.

Preferably, the solvent is selected from Miglyol® 840, Miglyol® 812 N, Miglyol® 829, Miglyol® 829 ECO, Miglyol® Coco 810, Miglyol® 810 N, Miglyol® 128, Miglyol® 808, Miglyol® T-C7, Miglyol® 8810, Miglyol® PPG 810, Miglyol® OE, Miglyol® DO, and Miglyol® 818, Abalyn®, limonene, benzyl benzoate, diethyl phthalate, isopropyl myristate, triethyl citrate, dipropylene glycol, and propylene glycol, triacetin, glycerin, 1,3 propanediol or combinations thereof, preferably Miglyol® 812 N.

Preferably, the solvent is a vegetable oil selected from coconut oil, corn oil, canola oil, cottonseed oil, olive oil, palm oil, peanut oil, rapeseed oil, safflower oil, sesame oil, soybean oil, and sunflower oil. Other examples of vegetable oils are given in the CTFA Cosmetic Ingredient Handbook, J. M. Nikitakis (ed.), 1st ed., The Cosmetic, Toiletry and Fragrance Association, Inc., Washington, 1988. A vegetable oil is an oil that comes from plant sources. Alternatively, the solvent is derived from a vegetable oil.

In preferred methods of the present invention, the active carrier phase is a fat or wax having a melting point of less than 60° C., preferably less than 45° C., preferably less than 25° C.

Preferably, the wax is selected from Softisan® 100, Softisan® 142, and Softisan® 154, or combinations thereof, preferably Softisan® 100.

Preferably the vegetable oil, fat or wax is present at a weight ratio of plant protein to active carrier phase is greater than 1:1, more preferably greater than 1.5:1, even more preferably greater than or equal to 2:1.

In the methods of the present invention, the method used to form the composition in step (c) is not particularly limited. For example, step (c) may involve membrane emulsification, mechanical stirring (e.g. low or high shear mechanical stirring), ultrasonication, high-shear mechanical stirring, high-pressure homogenisation and/or cavitation.

In preferred methods of the present invention, step (c) is conducted at a temperature in the range 30° C. to 50° C., preferably 35° C. to 45° C.

In preferred methods of the present invention, the composition formed in step (c) comprises droplets of said active ingredient(s) or said composition comprising the active ingredient(s) and an active carrier phase having a d₅₀ as determined by laser diffraction of 0.5 to 50 microns, preferably 1 to 40 microns, preferably 2 to 30 microns.

In preferred methods of the present invention, in step (b) or between step (b) and (c) or in step (c) an additive is added to the composition, preferably a water-soluble additive.

Preferably, said additive is selected from plasticisers, surfactants, rheology modifiers, opacifiers, preservatives, pigments, carbohydrates, gums, polymers, and nanoparticles, or mixtures thereof.

In preferred methods of the present invention, the additive is a plasticiser. Preferably, the additive is selected from glycerol, ethylene glycol, triethylene glycol, tetraethylene glycol, polyethylene glycol, propylene glycol, sorbitol, mannitol, xylitol, fatty acids, glucose, mannose, fructose, sucrose, lactic acid, citric acid, glycolic acid, malic acid, gluconic acid, tartaric acid, ethanolamine, urea, triethanolamine, vegetable oils, lecithin, waxes and amino acids.

In preferred methods of the present invention, the additive is a polysaccharide, preferably maltodextrin, starches or modified starches, such as Capsul® Starch or octenyl succinic anhydride (OSA)-modified starch. The use of polysaccharide additives allows for control over the composition of the matrix of the resultant microcapsule. For example, the use of polysaccharides means that the amount of plant-based protein(s) can be controlled.

In preferred methods of the present invention, the water-soluble additive is a water-soluble vitamin, preferably Vitamin C. The presence of additives, such as water-soluble vitamins, means that the final dried microcapsule is able to deliver the additive in addition to the encapsulated active ingredient(s) during end use, e.g. upon enzymatic degradation of the plant-based protein hydrogel matrix in the oral cavity or digestive tract of a human or animal.

In preferred methods of the present invention the weight ratio in the composition formed in step (c) of plant-based protein(s) to the composition comprising the active ingredient(s) and an active carrier phase is in the range 20:1 to 1:20.

In preferred methods of the present invention, the composition formed in step (c) is a homogenous composition.

In preferred methods of the present invention, the composition formed in step (c) is a two-phase system (e.g. an aqueous two-phase system).

In the methods of the present invention, step (d) involves drying the composition formed in step (c) to form a microcapsule or an agglomerate of microcapsules or larger bodies. A skilled person will be familiar with drying techniques and would know how to choose the necessary conditions to achieve dried microcapsules or larger bodies. Suitable drying techniques include spray drying as used herein, electrostatic spray drying, spray freeze drying and spray vacuum drying wherein the slurry is sprayed into a chamber under reduced pressure such that solvent can be removed at lower temperature, thus avoiding issues of thermal degradation. Advantageously, in the methods of the present invention, step (d) can be conducted at relatively low temperatures (e.g. at temperatures less than 100° C.). This aids the formation of small-sized microcapsules, and also prevents the loss or degradation of any volatile or heat-sensitive materials being encapsulated. Advantageously, in the methods of the present invention, step (d) can be conducted relatively quickly (e.g. residence time of seconds). This reduces the loss or degradation of any volatile or heat-sensitive materials being encapsulated.

Drying techniques that can be used either independently or in combination to evaporate the solvents from the composition of step (c) include spray-drying, fluidised bed drying, film drying, drum drying, belt drying, conduction drying, infrared drying or a combination thereof. The composition of step (c) can be dispersed and at least partially dried by spray-drying to form microcapsules which are then further dried by secondary processes, such as fluid-bed drying. The extended drying times typical of fluid-bed drying typically allow for lower drying temperatures to be used. Some designs of spray-dryer incorporate this two-stage drying process as an integral part of their design. Some fluid bed drying processes, which typically comprises spraying or dispersing the composition of step (c) onto or into a fluidised bed of particles, for example encapsulates of already dried composition (c), are preferred when larger encapsulates are required. The composition can be sprayed onto the fluidised bed or can be sprayed within the fluidised bed. Depending on process conditions, the composition spray will agglomerate and/or coat (and hence build up) passing microcapsules. One example of a fluidised bed coating and drying process is the Wurster-type of powder coater. The skilled person will be readily able to define conditions for control of encapsulate size from a fluid-bed drying process.

Other drying processes can be selected depending on the required form of the dried composition from step (d). Film drying, wherein the composition is cast as a thin film on a heated belt or drum, can be used to form aesthetic shapes comprising dried composition prepared in step (c). Belt drying, which can use conduction heating or infra-red heating, can be used in combination with other drying techniques to finish off the drying under controlled conditions.

During drying in step (d), the plant-based protein hydrogel slurry forms a plant-based protein carrier that encapsulates the active ingredient(s) in the form of a microcapsule. During drying in step (d), at least part of both the first and second co-solvent are evaporated to enable the microcapsule to form. This evaporation does not occur at the same rate for the two co-solvents so that during the process the slurry will typically become more concentrated in the least volatile co-solvent. The microcapsules formed during drying can still contain both first and second co-solvents trapped within the dry microcapsules and their ratio can be different to their starting ratio in the mixture in step (a).

In preferred methods of the present invention, the biodegradation percentage based upon O₂ consumption of the plant-based protein carrier as measured according to ISO-14851 version 2019 after 28 days is 60 to 100% based upon the ratio of the Biological Oxygen Demand (BOD) to the Theoretical Oxygen Demand, more preferably 65 to 10%, even more preferably 70 to 100%, even more preferably 75 to 100%, even more preferably 80 to 100%, even more preferably 85 to 100%, most preferably 90 to 100%. ISO-14851 version 2019 describes a method whereby the biological oxygen demand in a closed respirometer is used to determine the degree of biodegradation of materials in a natural aqueous environment. This is achieved by exposure of the material under lab conditions in an aqueous standard test medium to an inoculum from unadapted activated sludge that has not been pre-exposed. The measured value is calculated as a percentage of the theoretical oxygen demand calculated from the molecular formula. An internal reference of microcrystalline cellulose is also tested and the test is valid if its biodegradation % is greater than 60% at the end of the test.

In preferred methods of the present invention, the biodegradation percentage based upon 02 consumption of the microcapsules as measured according to ISO-14851 version 2019 after 28 days is 60 to 100% based upon the ratio of the Biological Oxygen Demand (BOD) to the Theoretical Oxygen Demand, more preferably 65 to 10%, even more preferably 70 to 100%, even more preferably 75 to 100%, even more preferably 80 to 100%, even more preferably 85 to 100%, most preferably 90 to 100%. ISO-14851 version 2019 describes a method whereby the biological oxygen demand in a closed respirometer is used to determine the degree of biodegradation of materials in a natural aqueous environment. This is achieved by exposure of the material under lab conditions 15 in an aqueous standard test medium to an inoculum from unadapted activated sludge that has not been pre-exposed. The measured value is calculated as a percentage of the theoretical oxygen demand calculated from the molecular formula. An internal reference of microcrystalline cellulose is also tested and the test is valid if its biodegradation % is greater than 60% at the end of the test.

In preferred methods of the present invention, step (d) involves electrostatic spray drying.

In preferred methods of the present invention, plant-based protein residues formed during step (d) but not incorporated into the microcapsule are recycled, preferably being added back into step (a).

In preferred methods of the present invention, the microcapsule has a d₅₀ as determined by laser diffraction of less than or equal to 250 μm, less than or equal to 200 μm, less than or equal to 150 μm, less than or equal to 100 μm, less than or equal to 50 μm, less than or equal to 30 μm, less than or equal to 10 μm.

In preferred methods of the present invention, the microcapsule has a diameter as determined by optical microscopy of less than or equal to 250 μm, less than or equal to 200 μm, less than or equal to 150 μm, less than or equal to 100 μm, less than or equal to 50 μm, less than or equal to 30 μm, less than or equal to 10 μm.

Preferred methods of the present invention further comprise subjecting the microcapsule to a post-treatment step. Preferably, the post-treatment step comprises a non-covalent cross-linking step, a covalent cross-linking step or a coating formation step.

In preferred methods of the present invention, the post-treatment step comprises a non-covalent cross-linking step. Preferably, the non-covalent cross-linking step comprises treating the microcapsule with a non-covalent cross linker selected from sodium tripolyphosphate (NaTPP), sodium hexametaphosphate, and phenolic compounds (e.g. tannic acid, caffeic acid etc.).

In preferred methods of the present invention, the post-treatment step comprises a covalent cross-linking step. Preferably, the covalent cross-linking step comprises treating the microcapsule with a covalent cross linker selected from genipin, epoxy compounds, glyceraldehyde, glutaraldehyde, formaldehyde, glyoxal, dialdehyde starch, microbial transglutaminase, and PolyCup® crosslinking resin, or combinations thereof.

In preferred methods of the present invention, the post-treatment step comprises a coating formation step.

In preferred methods of the present invention, the coating formation step comprises treating the microcapsule with a metal compound. Preferably, the metal compound is selected from a silver compound or a gold compound, preferably a silver compound.

In preferred methods of the present invention, the coating formation step comprises subjecting the microcapsule to a complex coacervation step using a polysaccharide. Preferably, the polysaccharide is selected from xanthan gum, gellan gum, and chitosan, or combinations thereof.

In preferred methods of the present invention, the coating formation step comprises treating the microcapsule with an aqueous mineral solution to form a mineral coating. Preferably, the aqueous mineral solution comprises iron salts, calcium salts, phosphate salts, carbonate salts, titanium salts or zinc salts, or combinations thereof.

In preferred methods of the present invention, the coating formation step comprises treating the microcapsule with a silicon-containing compound to form a silicon-based coating. Preferably, said silicon-containing compound is selected from sodium silicate, tetraethyl orthosilicate, tetrapropyl orthosilicate, dimethyl diethoxysilane and tetramethyl orthosilicate, or combinations thereof, more preferably wherein the sodium silicate is selected from sodium metasilicate, sodium orthosilicate and sodium pyrosilicate, with sodium metasilicate being most preferred. As will be understood by a skilled person, depending in which phase the silicon compound is present, the silicon-based coating can be formed on either the internal surface of the plant-based protein hydrogel (i.e. at the oil droplet interface) or the external surface of the plant-based protein hydrogel (i.e. to form a shell around the microcapsule). Water-soluble silicates are most preferred when coating the external surface of the plant-based protein hydrogel. Organic silicates are most preferred when formed on the internal surface of the plant-based protein hydrogel.

As will be understood by a skilled person, it is possible for multiple post-treatment steps to be performed. For example, in a preferred method of the present invention, the microcapsule is subjected to a non-covalent cross-linking step (e.g. using NaTPP) followed by a coating formation step (e.g. using chitosan), and optionally a further non-covalent cross-linking step (e.g. using NaTPP). For example, the microcapsule is subjected to an electrostatic/non-covalent cross-linking step using sodium tripolyphosphate (NaTPP) followed by a silica coating using poly-L-lysine as deposition aid. In a preferred method of the present invention, the microcapsule is subjected to a non-covalent cross-linking step using tannic acid followed by a coacervate coating formation step using xanthan gum.

Preferred methods of the present invention further comprise adding a flow aid to the microcapsule after step (d). Preferably, the flow aid is fumed silica. The addition of a flow aid can avoid clumping of the newly formed microcapsules.

Preferred methods of the present invention comprise further drying the microcapsule after step (d). Preferably, the further drying is selected from fluid bed drying and/or tray drying.

Preferred methods of the present invention further comprise re-suspending the microcapsule in an external phase, preferably an external aqueous phase, more preferably hard water or an acidic buffer solution.

Preferred methods of the present invention further comprise sieving the microcapsule, e.g. for size classification.

The present invention also provides a biodegradable microcapsule obtained by or obtainable by the method as hereinbefore described.

The present invention also provides a method for the preparation of a biodegradable microcapsule composition, the method comprising:
(a) preparing a biodegradable microcapsule according to the method as hereinbefore described; and
(b) suspending the biodegradable microcapsule in an external phase.

Preferably, said external phase is an external aqueous phase, preferably hard water or an acidic buffer solution.

Preferred methods of the present invention further comprise adding a suspending agent(s) to said external phase.

Preferably, the suspending agent(s) is selected from acacia gum, alginic acid, pectin, xanthan gum, gellan gum, carbomer, dextrin, gelatin, guar gum, hydrogenated vegetable oil category 1, aluminum magnesium silicate, maltodextrin, carboxymethyl cellulose, polymethacrylate, poly vinyl pyrrolidone, sodium alginate, starch, zein, water-insoluble cross-linked polymers such as cross-linked cellulose, cross-linked starch, cross-linked CMC, cross-linked carboxymethyl starch, cross-linked polyacrylate, and cross-linked polyvinylpyrrolidone, and expanded clays such as bentonite and laponite.

The present invention also provides a biodegradable microcapsule composition obtained by or obtainable by the method as hereinbefore described.

The present invention also provides a dried biodegradable microcapsule comprising an active ingredient(s) and a plant-based protein carrier comprising a plant-based protein(s), wherein the plant-based protein carrier encapsulates the active ingredient(s), and wherein the plant-based protein carrier has a solubility of less than 50% when measured at a protein concentration of 5% w/w in an aqueous solution at pH 7 and 25° C.

Protein solubility is determined using the following protocol: a known amount of spray-dried microcapsules is added to an aqueous solution, which is then centrifuged to separate the soluble and insoluble fractions. After centrifugation, the liquid supernatant (i.e. the soluble fraction) is removed without obtaining any of the solids that precipitate at the bottom (i.e. the insoluble fraction). The resultant supernatant is analysed for nitrogen content. Protein content in the supernatant is then calculated based on nitrogen content using the factor of 6.25. Protein solubility is defined as the amount of protein in the supernatant divided by the amount of protein in the whole aqueous solution.

In preferred biodegradable microcapsules of the present invention, the plant-based protein carrier has a solubility of less than 30% when measured at a protein concentration of 5% w/w in an aqueous solution at pH 7 and 25° C., more preferably a solubility of less than 10%.

In preferred biodegradable microcapsules of the present invention, at least 25%, more preferably at least 40%, even more preferably at least 50%, even more preferably at least 60% of the active ingredient(s) initially encapsulated remains present inside the microcapsule after incubation in water at 20° C. for 10 days, as determined by high-performance liquid chromatography (HPLC). In alternative preferred spray-dried biodegradable microcapsules of the present invention, at least 25%, more preferably at least 40%, even more preferably at least 50%, even more preferably at least 60% of the active ingredient(s) initially encapsulated remains present inside the microcapsule after incubation in water at 20° C. for 10 days, as determined by gas chromatography (GC).

More preferably, said water has been acidified such that it has a pH of less than 4.0, more preferably less than 3.0, more preferably less than 2.0, more preferably less than 1.0. Thus, the biodegradable microcapsules of the present invention are insoluble in water and are also stable under strongly acidic conditions. This has the advantage that the biodegradable microcapsules of the present invention can be used in a variety of applications in which such harsh conditions are prevalent. For example, the biodegradable microcapsules can be added to beverage formulations, which are generally acidic, in order to deliver an active ingredient (e.g. a vitamin or a mineral) to the consumer. The acid stability of the biodegradable microcapsules of the present invention also means that, once consumed, the microcapsules can survive the harsh acidic conditions of the stomach, such that the active ingredient(s) will not be released until it is in the small intestine and can be better absorbed. Thus, the biodegradable microcapsules of the present invention can provide controlled release of the encapsulated active ingredient(s). This is because the plant-based protein hydrogel matrix acts as an enteric coating.

In preferred biodegradable microcapsules of the present invention, the plant-based protein(s) is obtained from fava bean, mung bean, pea, rice, potato, rapeseed, lentil, chickpea, sunflower seed, pumpkin seed, flax, chia, canola, lupine, alfalfa, moringa, wheat, corn zein or sorghum; preferably the plant protein(s) is selected from pea protein, potato protein, rapeseed protein, lentil protein, chickpea protein, fava bean protein, mung bean protein, sunflower seed protein, pumpkin seed protein, flax protein, chia protein, canola protein, lupine protein, alfalfa protein, moringa protein and/or rice protein. More preferably, the plant-based protein is pea protein and/or potato protein. Such proteins are considered to be low allergenicity proteins.

In preferred biodegradable microcapsules of the present invention, the plant-based protein(s) has been pre-treated with an organic acid. Preferably, the organic acid is acetic acid, formic acid, propionic acid, an α-hydroxy acid and/or a β-hydroxy acid. Particularly preferably, the organic acid is acetic acid and/or lactic acid.

Preferred a-hydroxy acids include glycolic acid, lactic acid, acetic acid, malic acid, citric acid and/or tartaric acid, more preferably lactic acid or acetic acid. Preferred β-hydroxy acids include β-hydroxypropionic acid, β-hydroxybutyric acid, β-hydroxy β-methylbutyric acid, 2-hydroxybenzoic acid and carnitine.

In preferred biodegradable microcapsules of the present invention, the active ingredient(s) is selected from a vitamin, a mineral, a flavour material, a fragrance material, a pro-favour, a pro-fragrance, a flavour enhancer, a malodour counteractant, a nutraceutical, a live organism (e.g. a probiotic), a pharmaceutical, an anti-microbial agent, an anti-viral agent, an anti-inflammatory agent, a pesticide, a herbicide, a fertiliser, a fungicide, an insecticide, an animal repellent, an anti-acne agent, a skin lightening agent, an emollient, a skin moisturizing agent, a wrinkle control agent, a fabric softener active, a surface cleaning active, a skin conditioning agent, a hair conditioning agent, a dye, a pigment, and an adhesive, or combinations thereof.

In particularly biodegradable microcapsules of the present invention, the active ingredient(s) is at least one fragrance material or flavour material. Preferably, the at least one fragrance material or flavour material is selected from an alcohol, an aldehyde, a ketone, an ester, an ether, an acetate, an alkene, a nitrile, a nitrogenous heterocyclic compound, a sulfurous heterocyclic compound, and a Schiff base.

The fragrance materials and flavour materials employed in the present invention may be of natural origin (i.e. they are extracted from a natural source and are not synthetically modified in any way). Preferred fragrance materials or flavour materials of natural origin include nutmeg extract, cardamon extract, ginger extract, cinnamon extract, patchouli oil, geranium oil, orange oil, mandarin oil, orange flower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomile oil, clove oil, sage oil, neroli oil, labdanum oil, eucalyptus oil, verbena oil, mimosa extract, narcissus extract, jasmine extract, olibanum extract, rose extract, vanillin, coffee extract, hop oil or combinations thereof. Preferably, the fragrance materials or flavour materials of natural origin are plant-derived. The fragrance materials or flavour materials of natural origin may be used alone, or in combination, or in combination with synthetic fragrance materials.

In preferred biodegradable microcapsules of the present invention, the at least one fragrance material or flavour material has a vapour pressure greater than or equal to 0.00001 Torr at 25° C.

In preferred biodegradable microcapsules of the present invention, the at least one fragrance material or flavour material has a log P greater than or equal to 3.0, preferably greater than or equal to 3.5, more preferably greater than or equal to 4.0.

In preferred biodegradable microcapsules of the present invention, the at least one fragrance material or flavour material has at least two Hansen solubility parameters selected from: an atomic dispersion force (δD) from 14 to 20, a dipole moment (δP) of less than 8, and a hydrogen bonding (δH) from 2.5 to 11.

In preferred biodegradable microcapsules of the present invention, the at least one fragrance material or flavour material is part of a fragrance or flavour.

Preferably, the fragrance or flavour contains at least 20 wt % of fragrance material(s) or flavour material(s) with a log P greater than or equal to 3.0 based upon the total weight of the fragrance or flavour, more preferably greater than or equal to 3.5, more preferably greater than or equal to 4.0.

Preferably, the fragrance or flavour contains at least 40 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 50 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 60 wt % of fragrance material(s) or flavour material(s) with a log P greater than 3.0 based upon the total weight of the fragrance or flavour, more preferably greater than 3.5, more preferably greater than 4.0.

Preferably, the fragrance or flavour contains at least 10 wt % of fragrance material(s) or flavour material(s) of natural origin based upon the total weight of the fragrance or flavour, preferably at least 30 wt %, more preferably at least 50 wt %, more preferably at least 70 wt %.

Preferably, the fragrance or flavour contains at least 10 wt % of fragrance material(s) or flavour material(s) which have a biodegradation percentage based upon O2 consumption as measured according to ISO-14851 version 2019 after 28 days of 60 to 100% based upon the ratio of the Biological Oxygen Demand (BOD) to the Theoretical Oxygen Demand, more preferably 65%, even more preferably 70 to 100%, even more preferably 75 to 100%, even more preferably 80 to 100%, even more preferably 85 to 100%, most preferably 90 to 100%. Due to their low aqueous solubility, fragrance or 10 flavours can be added to the biodegradation test on an inert support according to the method in ISO 10634 version 2018: Water quality-Preparation and treatment of poorly water-soluble organic compounds for the subsequent evaluation of their biodegradability in an aqueous medium. In addition, the fragrance or flavour can be checked for any inhibitory effect on the microorganisms as specified in the method.

Preferably, the fragrance or flavour contains at least 80 wt %, preferably at least 90 wt %, more preferably at least 95 wt % of fragrance material(s) or flavour material(s) having at least two Hansen solubility parameters selected from: an atomic dispersion force (δD) from 14 to 20, a dipole moment (δP) of less than 8, and a hydrogen bonding (δH) from 2.5 to 11, based upon the total weight of the fragrance or flavour.

Preferably, the fragrance or flavour contains only low levels of materials with an alcohol functionality (e.g. a primary alcohol functionality. In preferred biodegradable microcapsules of the present invention, the fragrance or flavour comprises less than 40% wt alcohol-containing material based upon the total weight of the fragrance or flavour, more preferably less than 20% wt. In particularly preferred biodegradable microcapsules of the present invention, the fragrance or flavour does not comprise an alcohol-containing material.

Preferably, the fragrance or flavour material is of high odour impact. This is advantageous as it ensures that even low levels of fragrance are perceived when released from the microcapsules.

In alternative particularly preferred biodegradable microcapsules of the present invention, the active ingredient(s) is a vitamin or a mineral. Preferably, said active ingredient(s) is a vitamin or a mineral selected from Vitamin A, Vitamin B1, Vitamin B2, Vitamin B3, Vitamin B5, Vitamin B6, Vitamin B7, Vitamin B9, Vitamin B12, Vitamin C, Vitamin D, Vitamin E, Vitamin K, magnesium, sodium, potassium, zinc, iron, calcium, iodine, flaxseed oil, omega 3 fatty acid, folic acid, thiamin, riboflavin, niacin and phosphorous, or mixtures thereof. More preferably, said active ingredient is Vitamin D.

In alternative particularly preferred biodegradable microcapsules of the present invention, an additive can be included in the microcapsule core to act as an active protectant to reduce any potential degradation of the active ingredient(s) during processing and storage. Preferably, the core additive is an antioxidant or free radical scavenger such as Vitamin E and curcumin. Preferably, the core additive is a pigment such as carotenoids, preferably beta-carotene. Preferably, the core additive is a natural product with a mixture of antioxidants, such as polyphenols, and other compounds such as olive oil and turmeric.

The vitamins and minerals employed in the present invention may be sourced from a vitamin-containing and/or mineral-containing material. An example of such a material is seaweed.

In preferred biodegradable microcapsules of the present invention, the active ingredient(s) is part of a composition comprising the active ingredient(s) and an active carrier phase. Preferably, the active carrier phase is a solvent, a fat or a wax.

In preferred biodegradable microcapsules of the present invention, the active carrier phase is a solvent. Preferably, the solvent is a solvent with low volatility (e.g. having a vapour pressure of less than 0.1 Torr at 25° C., preferably less than 0.01 Torr at 25° C., preferably less than 0.001 Torr at 25° C.).

Preferably, the solvent has low or no odour.

Preferably, the solvent has at least two Hansen solubility parameters selected from: an atomic dispersion force (δD) of less than 20, a dipole moment (δP) of less than 8, and a hydrogen bonding (δH) of less than 11. More preferably, the solvent has at least two Hansen solubility parameters selected from: an atomic dispersion force (δD) of less than 20, a dipole moment (δP) of less than 4, and a hydrogen bonding (δH) of less than 5.

Preferably, the solvent has a density of greater than 1.07 g/cm³. Solvents having this property are advantageously able to prevent creaming of the encapsulates (e.g. in a final product formulation).

Preferably, the solvent contains only low levels of materials with an alcohol functionality (e.g. a primary alcohol functionality). In preferred spray-dried biodegradable microcapsules of the present invention, the solvent comprises less than 40% wt alcohol-containing material based upon the total weight of the solvent, more preferably less than 20% wt. In particularly preferred spray-dried biodegradable microcapsules of the present invention, the solvent does not comprise an alcohol-containing material.

In preferred biodegradable microcapsules of the present invention, the active carrier phase is a solvent selected from a carboxylic acid ester, a fatty acid ester, a phthalate ester, a triol, a diol, a rosin resin, an isoparaffin, a terpene, and a vegetable oil, or combinations thereof.

Preferably, the solvent is selected from Miglyol® 840, Miglyol® 812 N, Miglyol® 829, Miglyol® 829 ECO, Miglyol® Coco 810, Miglyol® 810 N, Miglyol® 128, Miglyol® 808, Miglyol® T-C7, Miglyol® 8810, Miglyol® PPG 810, Miglyol® OE, Miglyol® DO, and Miglyol® 818, Abalyn®, limonene, benzyl benzoate, diethyl phthalate, isopropyl myristate, triethyl citrate, dipropylene glycol, and propylene glycol, triacetin, glycerin, 1,3 propanediol or combinations thereof, preferably Miglyol® 812 N.

Preferably, the solvent is a vegetable oil selected from coconut oil, corn oil, canola oil, cottonseed oil, olive oil, palm oil, peanut oil, rapeseed oil, safflower oil, sesame oil, soybean oil, and sunflower oil. Other examples of vegetable oils are given in the CTFA Cosmetic Ingredient Handbook, J. M. Nikitakis (ed.), 1st ed., The Cosmetic, Toiletry and Fragrance Association, Inc., Washington, 1988. Alternatively, the solvent is derived from a vegetable oil.

In preferred biodegradable microcapsules of the present invention, the active carrier phase is a fat or a wax having a melting point of less than 60° C., preferably less than 45° C., preferably less than 25° C.

Preferably, the wax is selected from Softisan® 100, Softisan® 142, and Softisan® 154, or combinations thereof, preferably Softisan® 100.

In preferred biodegradable microcapsules of the present invention, the plant-based protein carrier further comprises an additive, preferably a water-soluble additive.

Preferably, said additive is selected from plasticisers, surfactants, rheology modifiers, opacifiers, preservatives, pigments, carbohydrates, gums, polymers, and nanoparticles, or mixtures thereof.

In preferred biodegradable microcapsules of the present invention, the additive is a plasticiser. Preferably, the additive is selected from glycerol, ethylene glycol, triethylene glycol, tetraethylene glycol, polyethylene glycol, propylene glycol, sorbitol, mannitol, xylitol, fatty acids, glucose, mannose, fructose, sucrose, lactic acid, citric acid, glycolic acid, malic acid, gluconic acid, tartaric acid, ethanolamine, urea, triethanolamine, vegetable oils, lecithin, waxes and amino acids.

In preferred biodegradable microcapsules of the present invention, the additive is a polysaccharide, preferably maltodextrin or OSA starch. The use of polysaccharide additives for allows control over the composition of the matrix of the resultant microcapsule. For example, the use of polysaccharides means that the amount of plant-based protein(s) can be controlled.

In preferred biodegradable microcapsules of the present invention, the water-soluble additive is a water-soluble vitamin, preferably Vitamin C. The presence of additives, such as water-soluble vitamins, means that the final microcapsule is able to deliver the additive in addition to the encapsulated active ingredient(s) during end use, e.g. upon enzymatic degradation of the plant-based protein hydrogel matrix in the oral cavity or digestive tract of a human or animal.

In preferred biodegradable microcapsules of the present invention, the microcapsule has a d₅₀ as determined by laser diffraction of less than or equal to 250 μm, less than or equal to 200 μm, less than or equal to 150 μm, less than or equal to 100 μm, less than or equal to 50 μm, less than or equal to 30 μm, less than or equal to 10 μm.

In preferred biodegradable microcapsules of the present invention, the microcapsule has a diameter as determined by optical microscopy of less than or equal to 250 μm, less than or equal to 200 μm, less than or equal to 150 μm, less than or equal to 100 μm, less than or equal to 50 μm, less than or equal to 30 μm, less than or equal to 10 μm.

In preferred biodegradable microcapsules of the present invention, the plant-based protein(s) have a protein secondary structure with at least 40% intermolecular β-sheet, at least 50% intermolecular β-sheet, at least 60% intermolecular β-sheet, at least 70% intermolecular β-sheet, at least 80% intermolecular β-sheet, or at least 90% intermolecular β-sheet, wherein the % intermolecular β-sheet content is measured by FTIR.

Preferred biodegradable microcapsules of the present invention do not contain or do not substantially contain a cross-linking agent.

In alternative preferred biodegradable microcapsules of the present invention, the plant-based protein carrier has been non-covalently modified by a non-covalent cross-linker. Preferably, the non-covalent cross linker is selected from sodium tripolyphosphate, sodium hexametaphosphate, and phenolic compounds (e.g. tannic acid, caffeic acid etc.).

In alternative preferred biodegradable microcapsules of the present invention, the plant-based protein carrier has been covalently modified by a covalent cross-linker. Preferably, the non-covalent cross-linker is selected from genipin, epoxy compounds, glyceraldehyde, glutaraldehyde, formaldehyde, glyoxal, dialdehyde starch, microbial transglutaminase, and PolyCup® crosslinking resin, or combinations thereof.

In preferred biodegradable microcapsules of the present invention, the plant-based protein carrier has a coating deposited thereon. Preferably, the coating is a metal coating, a coacervate coating or a mineral coating.

In preferred biodegradable microcapsules of the present invention, the metal coating is a silver coating or a gold coating, preferably a silver coating.

In preferred biodegradable microcapsules of the present invention, said coating is a silicon-based coating which is formed from a silicon-containing compound. Preferably, said silicon-containing compound is selected from sodium metasilicate, tetraethyl orthosilicate, tetrapropyl orthosilicate, dimethyl diethoxysilane and tetramethyl orthosilicate, or combinations thereof, preferably sodium metasilicate.

In preferred biodegradable microcapsules of the present invention, the coacervate coating is formed from a polysaccharide. Preferably, the polysaccharide is selected from xanthan gum, gellan gum, and chitosan, or combinations thereof.

In preferred biodegradable microcapsules of the present invention, the mineral coating is formed from an aqueous mineral solution. Preferably, the aqueous mineral solution comprises iron salts, calcium salts, phosphate salts, carbonate salts, titanium salts or zinc salts, or combinations thereof.

In preferred biodegradable microcapsules of the present invention, said coating is a polymeric coating, which is preferably formed from shellac.

Preferred biodegradable microcapsules of the present invention have a coating deposited on the covalently-modified or non-covalently modified plant-based protein matrix. Preferred features of the coatings are as described above.

In particularly preferred biodegradable microcapsules of the present invention, the plant-based protein is pea protein and the active ingredient is Vitamin D.

In preferred biodegradable microcapsules of the present invention, the biodegradation percentage based upon O₂ consumption of the plant-based protein carrier as measured according to ISO-14851 version 2019 after 28 days is 60 to 100%, based upon the ratio of the Biological Oxygen Demand (BOD) to the Theoretical Oxygen Demand, more preferably 65 to 100%, even more preferably 70 to 100%, even preferably 75 to 100%, even more preferably 80 to 100%, even more preferably 85 to 100%, most preferably 90 to 100%. ISO-14851 version 2019 describes a method whereby the biological oxygen demand in a closed respirometer is used to determine the degree of biodegradation of materials in a natural aqueous environment. This is achieved by exposure of the material under lab conditions in an aqueous standard test medium to an inoculum from unadapted activated sludge that has not been pre-exposed. The measured value is calculated as a percentage of the theoretical oxygen demand calculated from the molecular formula. An internal reference of microcrystalline cellulose is also tested and the test is valid if its biodegradation % is greater than 60% at the end of the test.

In preferred biodegradable microcapsules of the present invention, the biodegradation percentage based upon O₂ consumption of the microcapsule as measured according to ISO-14851 version 2019 after 28 days is 60 to 100% based upon the ratio of the Biological Oxygen Demand (BOD) to the Theoretical Oxygen Demand, more preferably 65 to 100%, even more preferably 70 to 100%, even preferably 75 to 100%, even more preferably 80 to 100%, even more preferably 85 to 100%, most preferably 90 to 100%. ISO-14851 version 2019 describes a method whereby the biological oxygen demand in a closed respirometer is used to determine the degree of biodegradation of materials in a natural aqueous environment. This is achieved by exposure of the material under lab conditions in an aqueous standard test medium to an inoculum from unadapted activated sludge that has not been pre-exposed. The measured value is calculated as a percentage of the theoretical oxygen demand calculated from the molecular formula. An internal reference of microcrystalline cellulose is also tested and the test is valid if its biodegradation % is greater than 60% at the end of the test.

The present invention also provides a composition comprising a biodegradable microcapsule as hereinbefore described and an external phase.

Preferably, said external phase is an external aqueous phase, preferably hard water or an acidic buffer solution.

Preferred compositions of the present invention further comprise a suspending agent(s) in the external phase.

Preferably, the suspending agent(s) is selected from acacia gum, alginic acid, pectin, xanthan gum, gellan gum, carbomer, dextrin, gelatin, guar gum, hydrogenated vegetable oil category 1, aluminum magnesium silicate, maltodextrin, carboxymethyl cellulose, polymethacrylate, poly vinyl pyrrolidone, sodium alginate, starch, zein, water-insoluble cross-linked polymers such as cross-linked cellulose, cross-linked starch, cross-linked CMC, cross-linked carboxymethyl starch, cross-linked polyacrylate, and cross-linked polyvinylpyrrolidone, and expanded clays such as bentonite and laponite.

The present invention also provides a formulated product comprising a biodegradable microcapsule as hereinbefore described.

The present invention also provides a method of making a formulated product, comprising:
(a) preparing a biodegradable microcapsule according to the method as hereinbefore described; and
(b) mixing said biodegradable microcapsule with a product formulation.

Preferably, the formulated product is a food, beverage, cosmetic, home care product, personal care product, pharmaceutical, industrial product (e.g. paint, adhesive, sandpaper, tape etc.), medical device, biomaterial, or agrochemical.

The present invention also provides the use of a biodegradable microcapsule as hereinbefore described in a formulated product.

The formulated product described in various aspects of the present invention may take any form suitable for use. Preferably, the formulated product is in the form of a vapour spray, aerosol, emulsion, lotion, liquid, cream, gel, stick, ointment, paste, mousse, powder, granular product, substrate, or semi-solid. More preferably the formulated product is a liquid, even more preferably an aqueous liquid.

### BRIEF DESCRIPTION OF THE FIGURES

FIGS. 1a and 1b are 10× optical microscope photographs of the spray-dried microcapsules of Examples 2B and 2E, respectively.

FIGS. 2a and 2b are 10× optical microscope photographs of the spray-dried microcapsules of Examples 4A and 4B, respectively.

FIG. 3 is a 10× optical microscope image of Example 5 control in water

FIGS. 4a and 4b are SEM images of the microcapsules of Example 6A at a magnification of ×80 and ×300, respectively.

FIGS. 5a, 5b and 5c are SEM images of the spray dried microcapsules of Example 6B at a magnification of ×60, ×500, and ×1250, respectively.

FIG. 6 is an SEM image of the spray dried microcapsules of Example 7 at a magnification of ×1200.

FIGS. 7a and 7b are 10× optical microscope images of the microcapsules of Example 7 before (FIG. 7a) and after (FIG. 7b) pressing them to release the oil.

FIGS. 8a, 8b and 8c are 10× microscope images of the concentrated slurry of Example 15A, 15B and Comparative Example 15C.

FIG. 9 is a 10× optical microscope image of the microcapsules of Example 20 in single strength citrate buffer.

FIGS. 10a and 10b are photographs of the inverted tubes of Example 21.

### EXAMPLES

### Materials

Pea Protein Isolate (PPI) (80% protein, 4 wt % carbohydrate'ProEarth P16109) was purchased from Cambridge Commodities Ltd.
Lactic acid (85%, Food Grade) was purchased from Fisher Scientific.
Acetic acid (glacial 99%) was purchased from Fisher Scientific.
Sodium benzoate was purchased from Fisher Scientific.
Vitamin D2 Oil (1 MIU/g) was purchased from Prinova Europe, UK.
Miglyol® 812N was purchased from IOI Oleochemical.
Softisan® 100 was purchased from IOI Oleochemical.
Vitamin D2 powder, Vitamin D2 100 CWS (100,000 IU/g), was supplied by Prinova Europe, UK.
Prinova GA (Gum Acacia) Vitamin D2 capsules sample was supplied by Prinova Europe, UK.
Robinsons Double Strength No Added Sugar Squash Apple & Blackcurrant (manufactured by Britvic Plc and commercially available from supermarkets in the United Kingdom).
MiWadi Single Concentrate Blackcurrant manufactured by Britvic Plc and commercially available from supermarkets in the Republic of Ireland.
Thymol was purchased from Fisher Scientific, UK
Calcium sulphate dihydrate (CaSO₄·2H₂O) was purchased from Fisher Scientific.
Magnesium sulphate dried (MgSO₄) was purchased from Fisher Scientific.
Potassium Chloride, Extra Pure, SLR, Eur. Ph., (KCl) was purchased from Fisher Scientific.
Sodium hydrogen carbonate, certified AR for analysis (NaHCO₃) was purchased from Fisher Scientific.
Potassium hydroxide (KOH) (>85%) was purchased from Sigma Aldrich.
Ethanol Absolute 99.8+%, (EtOH) certified AR for analysis was purchased from Fisher Scientific.
Maltodextrin was purchased from Sigma-Aldrich Gillingham, UK
Capsul® Starch was supplied by Ingredion, Manchester, UK
Polysorbate 80 was purchase from Sigma-Aldrich Gillingham, UK

All components for Phosphate buffered saline (PBS) (pH 7.4) were purchased from Fisher Scientific, UK, and were prepared in the following ratios: 138 mM NaCl, 2.7 mM KCl, 10 mM Na2HPO4, 2 mM NaH2PO4
Sodium acetate trihydrate was purchased from Alfar Aesar, UK
Calcium chloride dihydrate was purchased from Fisher Scientific, UK
Methylparaben was purchased from Alfar Aesar, UK
Gellan gum (high acyl) was purchased from Special Ingredients Ltd, UK
Flaxseed oil was purchased from Naissance, UK
Shellac was purchased from A.F. Suter & Company Ltd, UK.
Tannic acid was purchased from Fisher Scientific, UK
Xanthan Gum was purchased from CP Kelco, UK
Tetraethyl orthosilicate (TEOS) was purchased from Acros Organics, UK
Olive oil was purchased from a supermarket in the UK
Vitamin E was purchased as DL alpha-tocopherol 97+% from Fischer Scientific, UK
Curcumin was purchased as Curcumin 95% from Fisher Scientific
Beta-carotene was purchased as Synthetic beta-carotene ≥93% (UV) from Sigma Aldrich, USA

### Fragrance Materials:

| Common/trade name | CAS number | Supplier |
|---|---|---|
| Delta damascone | 57378-68-4 | Augustus Oils Limited and |
| | | Carvansons, UK |
| Geraniol BJ | 106-24-1 | Carvansons, UK |
| Undecavertol | 81782-77-6 | Carvansons, UK |
| Clonal (dodecane nitrile) | 2437-25-4 | Carvansons, UK |
| Dipentene | 138-86-3 | Carvansons, UK |
| Ortho tert.- butylcyclohexyl acetate | 88-41-5 | Carvansons, UK |
| (OTBCHA) | | |
| Diphenyl oxide | 101-84-8 | Carvansons, UK |
| Benzyl salicylate | 118-58-1 | Carvansons, UK |
| 2-[2-(4-Methyl-3-cyclohexen-1- | 95962-14-4 | Carvansons, UK |
| yl)propyl]cyclopentanone | | |
| (Peach cyclopentanone) | | |

### Standard Hard Water and Buffer Compositions:

### 1) Hard Water

This was prepared with the following concentrations of salts in deionised (DI) water: 192 mg/l of NaHCO₃, 120 mg/l CaSO4.2H2O, 120 mg/l MgSO4, 8 mg/l KCl. pH was adjusted to 3 with 1M HCl.

### 2) Single Strength Buffer

100 ml of single strength buffer solution was prepared as follows:
i. 1.904 g of citric acid monohydrate and 0.276 g of trisodium citrate dihydrate were weighed out;
ii. The mixture was completed to 80 g using DI water;
iii. The pH of the solution was adjusted to 3 using 1M HCl and 1M NaOH solutions, and the volume of the solution was completed to 100 g with DI water;
iv. 0.38 g of CaCl2 dihydrate was added; v. 0.2 g of sodium benzoate was added;
v. The solution was placed in an ultrasonic bath at 80° C. for 5-10 min (i.e. until complete dissolution was observed);
vi. The solution was allowed to cool down to 20° C.

### 3) Double Strength Buffer

100 ml of double strength buffer solution was prepared as for single strength buffer but with twice the level of all materials but with the mixture still being completed to 80 gs.

### Measurement Methods

### Vitamin D2 Analysis for Dry Powder Samples of Microcapsules: Version A

The vitamin D2 is extracted from the sample into dimethyl sulfoxide and then partitioned into hexane. The vitamin D2 is quantified using normal phase HPLC with UV detection. This method is suitable for the determination of vitamin D2 at high levels, typically from 35 iu/g to 500,000 iu/g.

### Vitamin D2 Analysis for Dry Powder Samples of Microcapsules: Version B

The powder sample (approx. 0.1 g) is suspended in 4 g of 50% w/v aqueous potassium hydroxide and 8 g of absolute ethanol. 0.1 g of sodium ascorbate is added. The sample is placed in an ultrasonic bath for 1 to 1.5 hours without heating, cooled on ice, then centrifuged at 14,500 rpm for 10 minutes before HPLC analysis on a reverse phase column using 100% methanol as eluent, and UV detection at 265 nm.

### Vitamin D2 Analysis for Dilute Liquid Systems Containing Microcapsules: Version A

The sample is cold saponified overnight with alcoholic potassium hydroxide (containing pyrogallol). The unsaponified fraction is separated by extraction into hexane. The extracted sample is then concentrated and injected onto a semi-preparative HPLC column. The fraction of the eluent containing the vitamin D2 is collected, evaporated to dryness, dissolved in methanol and the vitamin D2 is separated using reverse phase HPLC with UV detection. This method is suitable for determination of vitamin D2 at lower levels, typically from 0.5 iu/g to 100 iu/g. Higher levels can be quantified with further dilutions.

### Vitamin D2 Analysis for Dilute Liquid Systems Containing Microcapsules: Version B

The liquid sample (50 g) is centrifuged at 4500 rpm for 15 mins resulting in a sediment. To measure the concentration of Vitamin D2 in the supernatant about 5 g of supernatant is taken and 2 g of 50% w/v aqueous potassium hydroxide, 3 g of absolute ethanol and 0.05 g of sodium ascorbate are added, and the sample is mixed well. The sample is placed in an ultrasonic bath for 1 to 1.5 hours without heating, however by the end the temperature may have risen to 50-60° C. A microscope is used to check the microcapsules are fully broken, if not the mixture is sonicated for additional time. The sample is then cooled to room temperature using ice then about 1 ml is taken and centrifuged at 14,500 rpm for 15 minutes. The supernatant is transferred to an amber vial for HPLC analysis on a reverse phase column using methanol/water (95:5) as eluent, and UV detection at 265 nm. The Vitamin D2 content per 100 g of liquid is reported.

To measure the concentration of Vitamin D2 in the sediment the supernatant is carefully removed with a pipette leaving a small amount of the supernatant to avoid losing any sediment. This results in 1 to 2 g of wet sediment and the same steps as for the 5 g of supernatant are followed as described herein. This method is suitable for determination of encapsulated vitamin D2 at lower levels, typically from 0.5 iu/g to 100 iu/g. For shelf-life studies of encapsulated vitamin D2, only the sediment values are reported, since all vitamin D2 is found to remain within the microcapsules. All the vitamin D2 that has leaked from the microcapsule will have degraded.

### Hydrogel Slurry Particle Size

The particle size of the hydrogel was measured using an Anton Paar laser diffraction particle size analyser PSA 1190. Measurements were carried out by diluting the hydrogel within 1 h of finishing manufacturing in an aqueous solution with acetic acid or lactic acid adjusted to the same pH. The test material was diluted to the required concentration to ensure there are no agglomerate present and have the desired optical density (normally 5-15% obscuration) for the measurement. The d₅₀ quoted is for the volume distribution, as calculated via a general analysis using Mie theory.

### Droplet Size of Composition for Spray Drying

The droplet size of the oil or wax containing the active ingredient in the compositions used for spray drying was measured using an Anton Paar laser diffraction particle size analyser PSA 1190. Measurements were carried out by diluting the composition within 1 h of finishing manufacturing in an aqueous solution with acetic acid or lactic acid adjusted to the same pH. The test material was diluted to the required concentration to have the desired optical density (normally 5-15% obscuration) for the measurement. The d₅₀ quoted is for the volume distribution, as calculated via a general analysis using Mie theory.

### Protein Solids Content

The solids content of the hydrogel slurry was measured by the mass remaining on drying. Approximately 5 g of the hydrogel slurry was pipetted into a small polypropylene dish and the mass was accurately recorded. The dish was placed in a 40° C. oven overnight to dry. The dry mass was measured immediately after removing the dish from the oven and the solids content of the protein hydrogel slurry was calculated as a percentage of the initial wet mass.

### Viscosity of Composition for Spray-Drying

The viscosity of the compositions for spray drying were measured using an Anton Paar MCR 92 rheometer, within 1 h of finishing manufacturing the composition. The rheometer was set up using a cone (1 degree, 50 mm diameter) and plate geometry. The viscosity was measured at a temperature of 20° C. and a shear rate of 50/s.

### Spray-Dried Microcapsule Particle Size

The particle size of the final microcapsule powder was measured using an Anton Paar laser diffraction particle size analyser PSA 1190. The powder was added to reverse osmosis water diluted to the required concentration in order to have the desired optical density (normally 5-15% obscuration) for the measurement. The dispersion should be checked via an optical microscope for any larger agglomerates of microcapsules. If these are visible 0.5 wt % acetic acid can be added to ensure that the primary particles are well dispersed. The d₅₀ quoted is for the volume distribution, as calculated via a general analysis using Mie theory.

Alternatively, particle size can be measured using optical microscopy (e.g. using an open Frame microscope equipped with a CellCam 200CR camera, Aura Pro phase contrast illuminator and universal plan fluorite objectives at 4×, 10× and 20×). The microcapsule powder is added to either reverse osmosis water or single strength buffer as needed. In such a method, particle sizes are taken from the mean size measurements of 50 microcapsules, for each respective sample. The optical microscope is then calibrated using the grid of a Hirschmann counting chamber, (Fuchs Rosenthal). Using the straight line tool in ImageJ 1.53, particle diameters are measured from two center-edges with the overlay-text feature enabled to avoid repeating capsules.

### Scanning Electron Microscopy (SEM)

SEM images were obtained using a Hitachi TM3030.

### Optical Microscopy

Optical microscopy images were obtained using an open Frame microscope equipped with a CellCam 200CR camera, Aura Pro phase contrast illuminator and universal plan fluorite objectives at 4×, 10× and 20×. Samples were prepared for optical microscopy by first rehydrating the dried microcapsules in hard water having the following composition:

| | NaHCO₃ | CaSO₄•2H₂O | MgSO₄ | KCl |
|---|---|---|---|---|
| mg/l | 192 | 120 | 120 | 8 |

The microcapsules were left in the hard water or single strength buffer for 5 minutes before microscopy imaging. A cover slip was then placed on the top of the rehydrated sample, and images were taken. The slide was then removed from the microscope stage, and firm pressure was applied by hand to the cover slip before replacing the sample on the stage and taking images of the broken capsules.

### Total Fragrance and Fragrance Material Loading

Fragrance and fragrance material levels in the microcapsule can be determined by extraction of the fragrance and injection into a GC column. Quantification is achieved by use of a calibration curve for the given fragrance material diluted in ethanol.

To measure the total level of fragrance materials in an encapsulate sample, the capsules must first be broken by sonication. For example, 10-50 mg of dried encapsulate or 0.3-0.5 g of wet encapsulate collected on a 40 μm cell strainer in 3.0 g of deionised (DI) water and 10% KOH are vortexed to mix thoroughly. The solution is then sonicated with a Bandelin ultrasonicator, small probe TS104 for 1-2 minute at 30% amplitude (around 1.5-3.0 kJ in total). While sonicating, ice is used to keep the temperature below 20° C. to avoid any loss of fragrance through evaporation. An optical microscope as detailed above is used to check visually if the capsules are fully broken. If not, the sonication step is repeated. Ethanol is added to the mixture, which is then mixed and centrifuged. The supernatant is collected and retained as the first extraction. The residue is mixed with ethanol and centrifuged. The supernatant is collected and added to the first extraction. This mixture is then diluted as appropriate and injected into the GC for analysis.

For samples containing benzyl salicylate, this is quantified by measuring the benzyl alcohol formed from its hydrolysis.

### Total Thymol Loading

Thymol levels in the dried microcapsule was determined by extraction of the thymol and injection into a GC column as per the total fragrance loading method herein.

### Microcapsule Slurry Olfactive Evaluation

For any given encapsulate, the total fragrance level is quantified via GC as described herein. Based on the encapsulate loading the material is diluted to deliver the appropriate level of fragrance for an olfactive assessment. Optionally, preservatives and suspending agents are added to the dilute encapsulate sample.

50 μl of dilute encapsulate is pipetted using a low shear micropipette tip onto a standard microscope glass slide so that most of the surface area is covered, leaving the edges clear for handling the slide. This is repeated at least four times.

50 μl of dilute encapsulate slurry is also pipetted onto a rectangular fragrance evaluation card blotter (approx. 9 cm×5 cm). This is repeated at least in duplicate.

Slides and blotters were evaluated by trained evaluators at different points in time:
1) Immediately after application;
2) After being left overnight to dry at 20-25° C.;
3) After being left overnight to dry at 20-25° C. and then for glass slides rubbed with a gloved index finger with 10 strokes (in either direction);
4) After being left overnight to dry at 20-25° C. and then for glass slides pressed with another glass slide for 10 seconds and removed.
5) After being left overnight to dry at 20-25° C. and then for blotters rubbed with another clean blotter with 10 strokes (in either direction).

The evaluators graded the strength according to the following scale and provided comments on the character of the fragrance:

| Odour strength | Odour strength |
|---|---|
| 5 | Very strong |
| 4 | Strong |
| 3 | Moderate |
| 2 | Weak |
| 1 | Very weak |
| 0 | No odour |

### Moisture

The moisture content of microcapsules is measured by weighing a suitable mass of dried microcapsules into an Ohaus MB23 Moisture Analyzer (Ohaus Europe GmbH, Switzerland) and dispersing the sample evenly across the pan. The sample is heated to 120° C. and the moisture content reported as a weight %,

### Microcapsule Integrity

The physical integrity of microcapsules is assessed according to the following method. A sample of microcapsules (0.1 g) is weighed into a 15 ml falcon tube. Hot deionized water (>70° C.) is added to the capsules to a total of 10 g and hand shaken to redisperse the powder or pellet. The sample is then vortexed at 2000 rpm for 90 seconds to completely resuspend the microcapsules. The sample is checked for a uniform distribution by optical microscopy but, if required, can be vortexed for a further 30 seconds. Then the method for Vitamin D2 analysis for dilute liquid systems containing microcapsules: Version B given herein is followed for analysis of the supernatant and the sediment. Microcapsule integrity is reported as a % of Vitamin D2 that leaks into the supernatant. This is zero for complete microcapsule integrity.

### Example 1—Preparation of Protein Hydrogel Slurry with Acetic Acid

### Preparation of the Protein Hydrogel

800 ml of a mixture was prepared consisting of 12.5% (w/v) Pea Protein Isolate (PPI) in 30% (v/v) acetic acid solution.

The mixture was then heated in a water bath at 85° C., followed by a sonication step to disrupt large colloidal aggregates (Hielscher UIP), after which a transparent solution was obtained. The energy applied was 200 kJ.

The solution was then poured into a container to a depth of 1 cm and left to cool down at 5° C. for 28 hours to obtain a self-standing protein hydrogel.

### Application of Shear to Protein Hydrogel

Shear was then applied to the hydrogel as follows. The protein hydrogel was cut into ˜1 cm cubes via a low-shear cutting step. The cubes were placed inside a 75 micron filter bag, which was then submerged inside a bucket containing 10 L of reverse osmosis water. This formed a coarse protein hydrogel slurry within the filter bag. The hydrogel cubes were left to soak, with occasional gentle agitation. This step was performed to reduce the concentration of acetic acid in the hydrogel by diffusion to the continuous aqueous phase.

The strained gel cubes were transferred into a bottle with 4 wt % sodium benzoate added and were exposed to high shear followed by ultra-high shear (probe sonication with a Hielscher sonicator to 200 kJ) on ice so as to form a homogeneous low-viscosity protein hydrogel slurry.

### Example 2—Preparation of Spray-Dried Microcapsules Containing Vitamin D2

### Preparation of Wax/Vitamin D2 Mixtures

A 22 wt % (220,000 IU/g) and a 11 wt % (110,000 IU/g) mixture was made of Vitamin D2 oil (1 MIU/g) in Softisan® 100 wax. The wax was melted above 40° C., the vitamin oil added, and the mixture homogenised by hand shaking. The mixture was held above 40° C. until required.

### Example 2A: 5:1 Ratio of Acid-Treated Hydrogel Slurry Solids to Wax, with Vitamin D2 Added by Sonication

1500 g of the protein hydrogel slurry was made as per Example 1, wherein the PPI and acetic acid mixture was heated for 20 minutes, and the gel cubes were soaked for 2 hours, then high sheared at 15,000 rpm for 15 minutes and then ultra-high sheared at 0.25 kJ/ml to form the hydrogel slurry.

### Preparation of Composition

The protein hydrogel slurry was heated to 35° C. and the 22 wt % wax/vitamin D2 molten mixture was added, such that a 98.3:1.7 by weight ratio of protein hydrogel slurry to wax/Vitamin D2 was achieved. The resultant mixture was mechanically high sheared using an Ultra-Turrax disperser at 15,000 rpm for 10 minutes, followed by ultra-high shearing on ice using a Hielscher sonicator applying energy of 0.1 kJ/ml. This resulted in a composition having a viscosity of 27.3 cP, and a mean droplet size, d₅₀, of 0.67 microns.

### Spray-Drying of Composition

The composition was spray dried according to the process settings in Table 1, resulting in microcapsules having a mean particle size, d₅₀, of 23.8 microns. The Vitamin D2 concentration in the microcapsule powder was measured using the method version A herein described and was found to be 100,250 ug/100 g.

### Example 2B: 2:1 Ratio of Acid-Treated Hydrogel Slurry Solids to Wax, with Vitamin D2 Added by Mechanical Mixing

1500 g of the protein hydrogel was made as per Example 1 wherein the PPI and acetic acid mixture was heated for 20 minutes and the gel cubes were soaked for 2 hours, then high sheared at 15,000 rpm for 15 minutes and then ultra-high sheared at 0.25 kJ/ml to form the hydrogel slurry.

### Preparation of Composition

The protein hydrogel was heated to 35° C. and the 11 wt % wax/vitamin D2 molten mixture was added, such that a 96:4 by weight ratio of protein hydrogel slurry to wax/Vitamin D2 was achieved. The resultant mixture was mechanically high sheared using an Ultra-Turrax disperser at 15,000 rpm for 10 minutes. This resulted in a composition with a viscosity of 31.4 cP, and a mean droplet size, d₅₀, of 4.6 microns.

### Spray-Drying of Composition

The slurry was spray dried according to the process settings in Table 1 resulting in microcapsules having a mean particle size, d₅₀, of 35.9 microns. The Vitamin D2 concentration in the microcapsule powder was measured using the method version A herein described and was found to be 94,750 ug/100 g.

### Example 2C: 5:1 Ratio of Acid-Treated Hydrogel Slurry Solids to Wax, with Vitamin D2 Added by Mechanical Mixing

1500 g of the protein hydrogel slurry was made as per Example 1, wherein the PPI and acetic acid mixture was heated for 20 minutes and the gel cubes were soaked for 2 hours, then high sheared at 15,000 rpm for 15 minutes and then ultra-high sheared at 0.25 kJ/ml to form the hydrogel slurry.

### Preparation of Composition

The protein hydrogel slurry was heated to 35° C. and the 22 wt % wax/vitamin D2 molten mixture was added, such that a 96:4 by weight ratio of protein hydrogel slurry to wax/Vitamin D2 was achieved. The mixture was mechanically high sheared using an Ultra-Turrax disperser at 15,000 rpm for 10 mins. This resulted in a composition with a viscosity of 35.8 cP, and a mean droplet size, d₅₀, of 6.2 microns.

### Spray-Drying of Composition

The composition was spray dried according to the process settings in Table 1. Two different settings were used, yielding samples Ci and Cii. The Ci and Cii microcapsules were each measured to have a mean particle size, d₅₀, of 31.3 microns. The Vitamin D2 concentration in the Cii microcapsule powder was measured using the method version A herein described and was found to be 95,500 μg/100 g.

### Example 2D: 5:1 Ratio of Protein Solids in Sonicated PPI (in 5 wt % Acetic Acid) to Wax, with Vitamin D2 Added by Mechanical Mixing

### Preparation of PPI Mixture

A mixture of 8 wt % pea protein isolate in 5 wt % (v/v) acetic acid solution was prepared so that the solids concentration would be comparable to the protein hydrogel slurry prepared in Example 1. The mixture was ultra-high sheared using a Hielscher probe sonicator to an energy input of 1 kJ/ml. This yielded a protein mixture with a mean particle size, d₅₀, of 2.9 microns. The pH of the protein mixture was measured as 3.4.

### Preparation of Composition

The protein mixture was heated to 35° C. and the 22 wt % wax/vitamin D2 molten mixture was added, such that a 98.3:1.7 by weight ratio of protein mixture to wax/Vitamin D2 was achieved. The mixture was mechanically high sheared using an Ultra-Turrax disperser at 15,000 rpm for 10 minutes. This resulted in a composition having a viscosity of 5.0 cP, and a mean droplet size, d₅₀, of 4.4 microns.

### Spray-Drying of Composition

The composition was spray dried according to the process settings in Table 1 resulting in microcapsules with a mean particle size, d₅₀, of 20.2 microns. The Vitamin D2 concentration in the microcapsule powder was measured using the method version A herein described and was found to be 86,500 μg/100 g.

### Comparative Example 2E: 5:1 Ratio of Protein Solids in Sonicated PPI to Wax, Vitamin D Added by Mechanical Mixing

### Preparation of PPI Mixture

A slurry of 8 wt % pea protein isolate in reverse osmosis water was prepared so that the solids concentration would be comparable to the protein hydrogel slurry prepared in Example 1. The mixture was ultra-high sheared using a Hielscher probe sonicator to an energy input of 1 kJ/ml. This yielded a protein mixture with a mean particle size, d₅₀, of 1.4 microns. The pH of the protein mixture was measured as 6.85.

### Preparation of Composition

The protein mixture was heated to 35° C. and the 22 wt % wax/vitamin D2 molten mixture was added, such that a 98.3:1.7 by weight ratio of protein mixture to wax/Vitamin D2 was achieved. The mixture was mechanically high sheared using an Ultra-Turrax disperser at 15,000 rpm for 10 minutes. This resulted in a composition with a viscosity of 4.9 cP, and a mean droplet size, d₅₀, of 4.6 microns.

### Spray-Drying of Composition

The composition was spray dried according to the process settings in Table 1 resulting in microcapsules with a mean particle size, d₅₀, of 40.0 microns. The Vitamin D2 concentration in the microcapsule powder was measured using the method version A herein described and was found to be 93,000 μg/100 g.

### Spray Drying Conditions for Examples 2A to 2E

All the compositions prepared in Examples 2A to 2E were spray dried in a co-current spray dryer with a 1 m diameter chamber and a Spraying Systems FloMax nozzle. The inlet air flow was maintained at 1.5 kg/min and the slurry feed addition rate was adjusted within the specified range to maintain the outlet temperature. The feed properties and spray drying conditions are summarised in Table 1 below.

TABLE 1

| | Weight | | | | | Feed | | |
|---|---|---|---|---|---|---|---|---|
| | ratio of | | Viscosity | Feed | Atomising | addition | Inlet | |
| | protein | % | of feed | droplet | air | rate | air | Air out |
| | to | Solids | at 50/s | size | pressure | litres/ | temp | temp |
| Sample | VitD/wax | in feed | (cP) | (microns) | (psi) | hour | (° C.) | (° C.) |
| 2A | 5:1 | 10.5% | 27.28 | 0.67 | 40 | 3.3- | 160 | 80 |
| | | | | | | 3.5 | | |
| 2B | 2:1 | 12.4% | 31.4 | 4.57 | 40 | 4.5 | 160 | 80 |
| 2Ci | 5:1 | 10.4% | 35.82 | 6.16 | 40 | 3.3- | 180 | 90 |
| | | | | | | 3.5 | | |
| 2Cii | 5:1 | 10.4% | 35.82 | 6.16 | 40 | 3.3- | 160 | 80 |
| | | | | | | 3.5 | | |
| 2D | 5:1 | 9.6% | 4.95 | 4.37 | 65 | 3.3- | 160 | 80 |
| | | | | | | 3.5 | | |
| 2E | 5:1 | 9.6% | 4.93 | 4.62 | 40 | 3.5 | 160 | 80 |

The conditions in Table 1 demonstrate that despite the higher feed viscosities of Examples 2A, 2B and 2C, compared to the Examples 2D and 2E, the compositions were successfully spray dried with standard equipment and process conditions. This is thought to be due to the shear thinning nature of the compositions of Examples 2A, 2B and 2C.

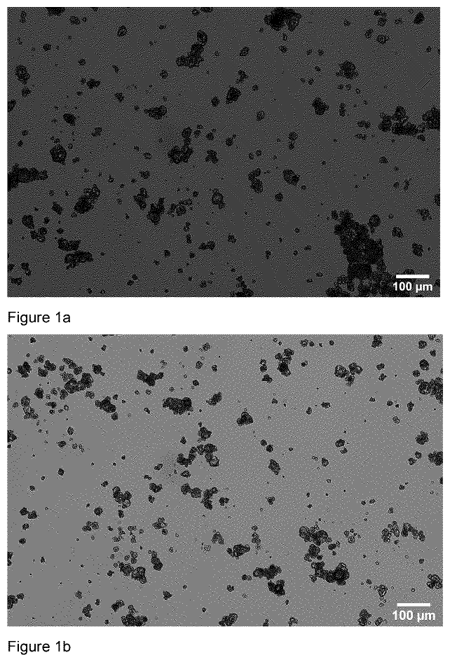

All compositions were successfully spray dried producing free flowing powders with a mean particle size, d₅₀, between 20 and 40 microns. FIGS. 1a and 1b are 10× optical microscope photographs of the spray-dried microcapsules of Examples 2B and 2E, respectively, showing the visual similarity of the microcapsule powders produced.

### Example 3—Preparation of Protein Hydrogel Slurry with Lactic Acid

### Preparation of the Protein Hydrogel

50.0 g pea protein isolate was added to 212.5 g of reverse osmosis water in a bottle. The bottle was shaken to disperse the protein and 187.5 ml of 85% (v/v) lactic acid was mixed in. The mixture was placed in an 85° C. water bath for 30 minutes and then ultrasonicated with a Bandelin sonicator until 100 kJ had been delivered. The hot protein solution was poured into a petri dish and allowed to cool at 5° C. overnight to obtain a self-standing protein hydrogel.

### Application of Shear to Protein Hydrogel

The hydrogel was cut into 1 cm cubes and placed in a 75 micron nylon mesh bag in a bucket. 5 L of reverse osmosis water was added and the cubes were left to soak for approximately 1.5 hours, with occasional gentle agitation. The water was then drained from the bag and the same soaking procedure repeated three more times, until the pH was above 3.0.

The hydrogel was then drained and added to a solution of 5 wt % of sodium benzoate at 20 mls per kilo of gel (with the pH adjusted to 4-5 using lactic acid). The mixture was sonicated using a Bandelin sonicator until 0.1 kJ/ml had been applied. The solids content of the hydrogel slurry was measured to be 5.1 wt % by the method described herein.

### Example 4—Preparation of Spray-Dried Microcapsules Containing Vitamin D2

### Preparation of Composition

Vitamin D2 oil (1 MIU/g) was diluted in Miglyol® 812N or Softisan® 100 wax to a concentration of 6000 ug/g. This concentration was chosen to target the manufacture of spray dried particles containing a Vitamin D2 concentration of 1,000 ug/g. In the case of Softisan® 100 wax, the wax was melted above 40° C., the vitamin oil added, and the mixture homogenised by hand shaking. The mixture was held above 40° C. until required.

The required mass of oil or wax and Vitamin D2 mixture was then added to the protein hydrogel slurry made in Example 3 to achieve a mass ratio of 5:1 hydrogel slurry solids to Vitamin D2: wax/oil mixture. A composition was formed by mixing with a IKA Ultra-Turrax for 15 minutes at 5000 rpm, followed by probe sonication to 0.1 kJ/ml with a Bandelin sonicator.

### Spray Drying Conditions

The spray drying was performed using a ProCept spray dryer in its three-column set-up. This gave a column height of 1.8 m with a column diameter of 0.15 m. The two-fluid nozzle was used with a tip size of 0.8 mm. The atomisation air flow rate was 10 l/min. The fluid was pumped into the spray dryer with a syringe pump at 6 ml/min. Other settings are given in Table 2 below. An additional air flow of 100 l/min was set for the cyclone.

TABLE 2

| | | Viscosity | Inlet air | Inlet air | Chamber | Air out |
|---|---|---|---|---|---|---|
| | | at 50/s | flow rate | temp | out temp | temp |
| Example | Sample type | (cP) | (m³/min) | (° C.) | (° C.) | (° C.) |
| 4A | Miglyol ®812N | 57.6 | 0.6 | 140 | 79 | 69 |
| | oil/VitD2 | | | | | |
| 4B | Softisan ® | 58.1 | 0.4 | 138 | 68 | 66 |
| | 100 | | | | | |
| | Wax/VitD2 | | | | | |

The results demonstrate that protein hydrogel slurries formed using lactic acid can also be spray dried using standard equipment and under standard conditions. The results also demonstrate that a range of different carrier materials for the active ingredient, here Vitamin D2, can be employed (e.g. oils and waxes).

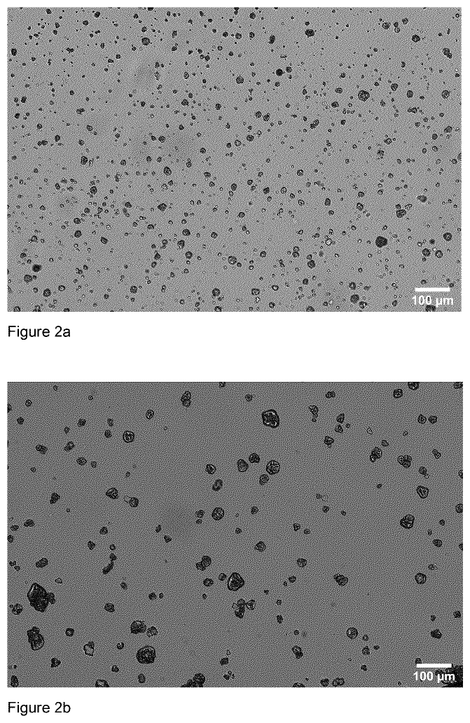

The spray drying experiments produced two free flowing powders with a mean particle size, d₅₀, of 15.8 microns for Example 4A and 32.5 microns for Example 4B measured by laser diffraction. The Vitamin D2 concentration in the microcapsule powder was measured using the method version A described herein and was found to be 89,000 μg/100 g and 86,500 μg/100 g in samples 4A and 4B, respectively. FIGS. 2a and 2b are 10× optical microscope photographs of the spray-dried microcapsules of Examples 4A and 4B, respectively, showing the visual similarity of the powders produced where the difference in particle size can be observed.

### Example 5—Accelerated Stability Testing of Vitamin D2 Microcapsules in a Beverage Formulation

### Preparation of Samples

The microcapsules of Examples 4A and 4B were each added to Robinsons Double Concentrate No Added Sugar Apple & Blackcurrant squash. More specifically, 45 mg of microcapsules were added to 1 litre of squash, to give a vitamin D concentration of 4.5 ug/100 ml in the product, on the assumption that the vitamin D concentration in the microcapsules was 100,000 μg/100 g. A comparative example was also prepared by adding 9 mg of Prinova Vitamin D2 powder to a sample of the same squash. Prinova Vitamin D2 powder is a commercially available powdered form of Vitamin D2. The three mixtures were heated to 95° C. on a hotplate, with constant stirring, and then held at 95° C. for 30 seconds to mimic the temperature profile during flash pasteurization. The mixtures were then allowed to cool to 20° C.

### Accelerated Stability Testing

200 ml transparent plastic PET bottles were filled with each of the three squash samples and placed in a Binder KBF P 240-230 V chamber, which was set for accelerated ambient light stability studies, in its standard configuration with visible and UV-A tubes. The nominal UV-A output was 1.1 W/m². The temperature was set to 20° C. and lab humidity was used (typically 50% relative humidity (RH)). Initial Vitamin D2 loading in the squash was measured using the method version A herein described and then again after 4 weeks in the Binder chamber. The % reduction in Vitamin D2 content over 4 weeks was calculated and is recorded in Table 3. Vitamin D2 is expected to degrade significantly on exposure to UV over this time frame.

TABLE 3

| | | Vitamin D2 reduction | |
|---|---|---|---|
| Example | Powder Description | from Week 0 to Week 4 | Vitamin D carrier |
| Comparative | Prinova Vit D2 powder | 95% | Unknown, likely |
| | | | carbohydrate |
| 4A | Spray dried Vit D2 | 54% | Miglyol ® 812N |
| | encapsulate with oil core | | |
| 4B | Spray dried Vit D2 | 0% (no reduction | Softisan ® 100 |
| | encapsulate with wax core | within method | |
| | | measurement error | |
| | | of +/− 18%) | |

As can be seen from Table 3, after 4 weeks in the squash the commercially available powdered Vitamin D2 degrades almost completely with 95% of the initial Vitamin D2 degraded.

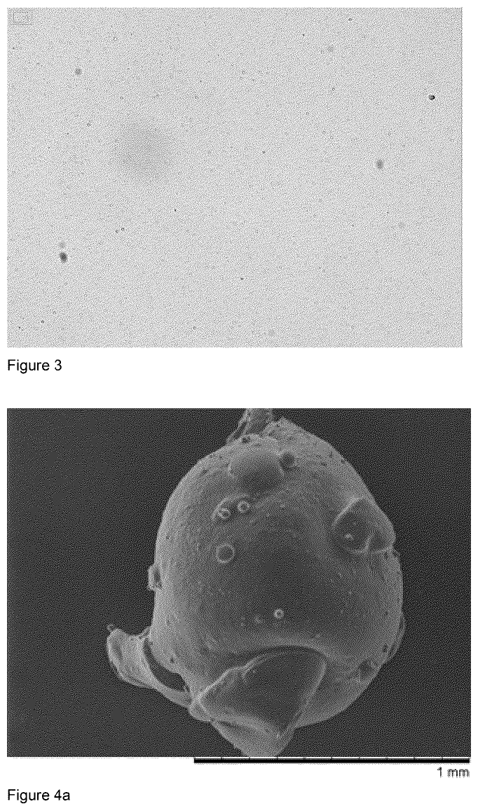

FIG. 3 is a 10× microscope image of the Prinova Vitamin D2 powder added to cold tap water showing the presence of a fine dispersion of material and no microcapsules. Without wishing to be bound by theory, it is believed that the carrier material in this powder is highly soluble in the squash so that it disintegrates in water and so is not able to protect the Vitamin D2 in accelerated stability testing.

However, for Example 4A, after 4 weeks in the squash the Vitamin D2 level is still approximately half of the original level, demonstrating that this microcapsule having a plant-based protein carrier and an oil-based active carrier phase has protected it from exposure to UV and hence reduced degradation. The presence of an insoluble plant-based protein carrier clearly helped to protect the encapsulated Vitamin D2 compared to the commercially available material tested in the comparative experiment. Furthermore, for Example 4B, after 4 weeks in the squash the Vitamin D2 level is substantially unchanged from the original level, demonstrating that this microcapsule having a plant-based protein carrier and a wax-based active carrier phase was able to fully protect the Vitamin D2 from degradation. Without wishing to be bound by theory, it is thought that the use of a solid wax carrier for the Vitamin D2 means the material surrounding the vitamin is more opaque and therefore provides even better protection for the vitamin from UV.

### Example 6—Preparation of Spray Dried Fragrance Microcapsules

### Preparation of Fragrance/Solvent Mixture

A mixture was prepared of delta-damascone and Miglyol® 812 in the weight ratio 80:20 by gentle mixing.

### Preparation of Spray Dried Microcapsules

The protein hydrogel slurry was made as per Example 1, wherein the PPI and acetic acid mixture was heated for 30 minutes, the gel cubes were soaked for 1 hour then high sheared at 25,000 rpm for 15 mins using an IKA Ultra-Turrax and then ultra-high sheared using sonication (Hielscher sonicator) to 1.0 kJ/ml to form the hydrogel slurry.

The mean particle size of the hydrogel slurry, d₅₀, was measured according to the method herein described and found to be 2.5 microns. The mean solids content of the hydrogel slurry was measured according to the method herein described and was found to be 8.6 wt %.

Two samples were prepared with the compositions shown in Table 4 by pouring the fragrance/solvent mixture into the hydrogel slurry and mixing with an Ultra-Turrax at 15,000 rpm for 5-15 seconds to achieve the desired dispersed droplet size of 5 μm.

TABLE 4

| Material (wt %) | Example 6A | Example 6B |
|---|---|---|
| Pea Protein | 4.9 | 5.4 |
| Delta-damascone | 36.7 | 32.2 |
| Miglyol ®812N | 12.4 | 10.9 |
| Water | 46.0 | 51.5 |
| Ratio PPI:Oil phase (fragrance + | 1:12.5 | 1:8 |
| Miglyol) | | |

Example 6A phase easily separated to give a clear liquid top layer and a bottom layer of hydrogel fragments, but this could be made homogenous again with vigorous mixing prior to spray drying. The well mixed slurry had a viscosity of 150 cP at 50/sec.

A 1.5 mg droplet of Example 6A was suspended on a fine wire connected to a microbalance and positioned in the middle of a pipe of internal diameter 2.5 cm. Air at a temperature of 97° C. and a velocity of 0.85 m/s was passed around the droplet and the weight of the droplet was recorded over 11 minutes. At the end of the experiment, the dried droplet was carefully detached from the wire and positioned on a Hitachi TM3030 SEM mounting pad for examination.

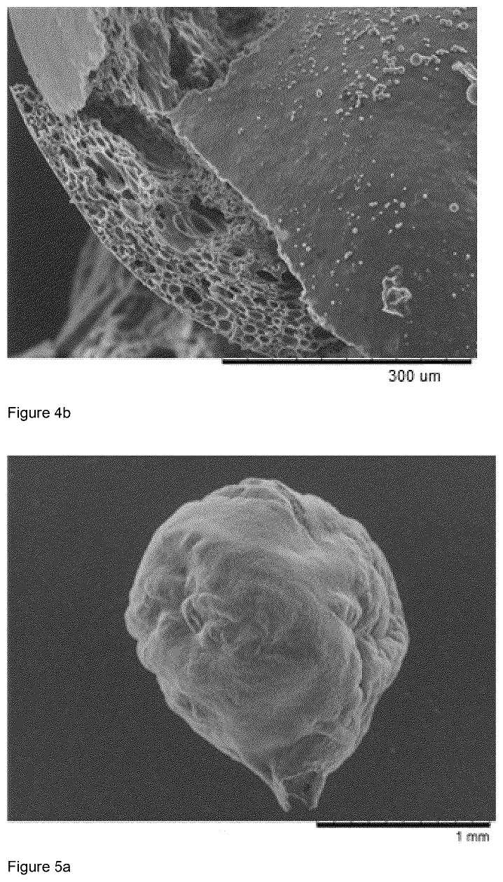

The resulting particle had an internal matrix structure and an external smooth skin as seen in the SEM images of FIGS. 4a and 4b.

Example 6B was a homogeneous, viscous and semi-solid with a viscosity of 1000 cP at 50/sec.

A 2.5 mg droplet of Example 6B was suspended on a fine wire connected to a microbalance and positioned in the middle of a pipe of internal diameter 2.5 cm. Air at a temperature of 85° C. and a velocity of 0.85 m/s was passed around the droplet and the weight of the droplet was recorded over 3.5 minutes. At the end of the experiment, the dried droplet was carefully detached from the wire and positioned on a Hitachi TM3030 SEM mounting pad for examination.

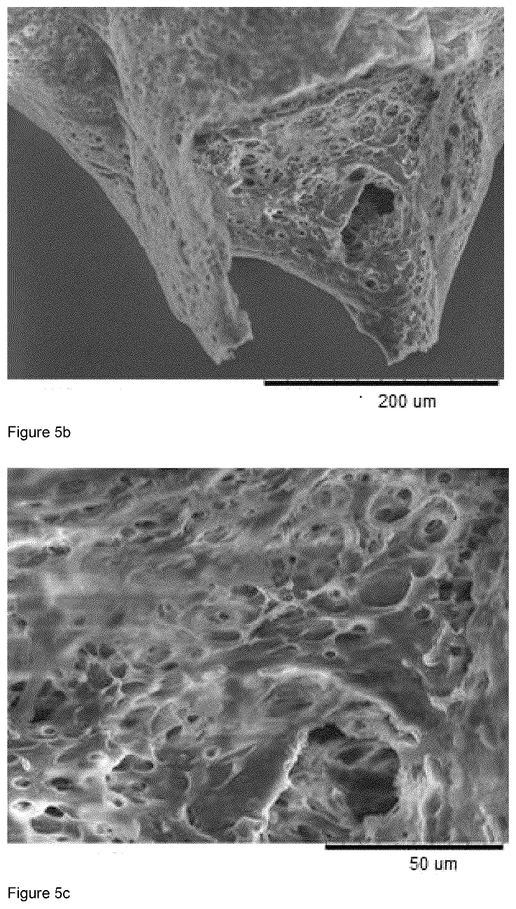

The resulting particle had a matrix structure and porous surface as can be seen in the SEM images of FIGS. 5a, 5b, and 5c.

### Example 7-Preparation of Spray Dried Fragrance Microcapsules

### Preparation of Fragrance/Solvent Mixture

A mixture was prepared of delta-damascone and Miglyol® 812 in the volume ratio 80:20 by gentle mixing.

### Preparation of Spray Dried Microcapsules

The protein hydrogel slurry was made as per Example 1. The hydrogel slurry was diluted with acetic acid to a mean protein solids content of 8.0 wt %. A 221 g batch was stirred with an UltraTurrax lab mixer at 8000 rpm for 2 minutes. 131 g of the fragrance: oil mixture containing 80% delta-damascone and 20% Miglyol® 812 oil was then added. The composition is given in Table 5 below.

TABLE 5

| Material (wt %) | Example 7 |
|---|---|
| Pea Protein | 5.0 |
| Delta-damascone | 29.8 |
| Miglyol ® 812N | 7.4 |
| Water (acetic acid content approx. 3%) | 57.8 |
| Ratio PPI:Oil phase (fragrance + Miglyol) | 1:7.4 |

The batch was subjected to high shear using an UltraTurrax lab mixer set to 8000 rpm for 5 minutes to make a fine emulsion. It was expected that the droplet size was around 5 microns.

The sample was then spray-dried using a ProCepT R&D Spray Dryer in its three-column set-up. This gave a column height of 1.8 m with a column diameter of 0.15 m. The air inlet temperature was 130° C. at a flowrate of 550 l/min. The two-fluid nozzle was used with a tip size of 1.0 mm. The atomisation air flow rate was 10 l/min. The fluid was pumped into the spray dryer with a syringe pump at 6 ml/min. An additional air flow of 140 l/min was introduced below the drying chamber to convey air to the cyclone.

The dried microcapsule powder formed from Example 7 was collected from the cyclone and also from the internal surfaces of the equipment where it had stuck, for example in the transfer pipe to the cyclone.

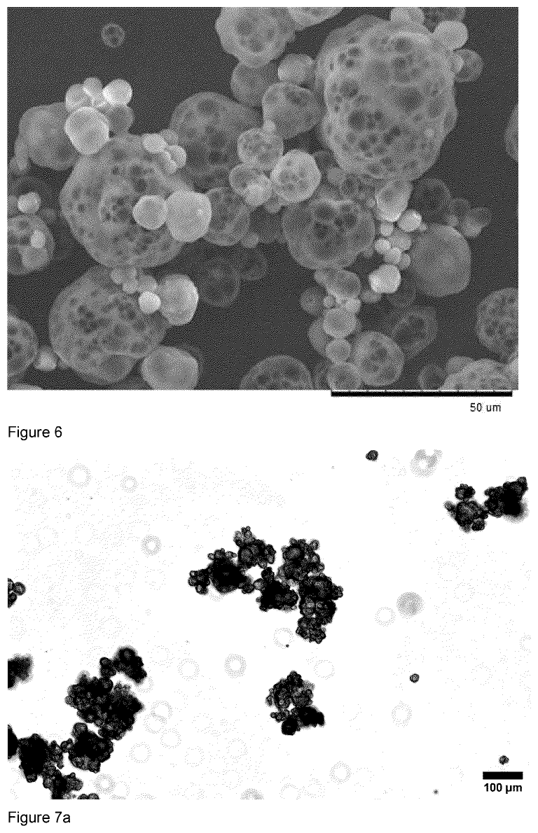

The microcapsules of Example 7 were examined under a Hitachi TM3030 SEM and mainly spherical fragrance encapsulates of diameter around 30 μm or less were observed (FIG. 6). The microcapsules appear to have a pock-marked surface, indicating that there was minimal free fragrance present and that the samples were reasonably dry.

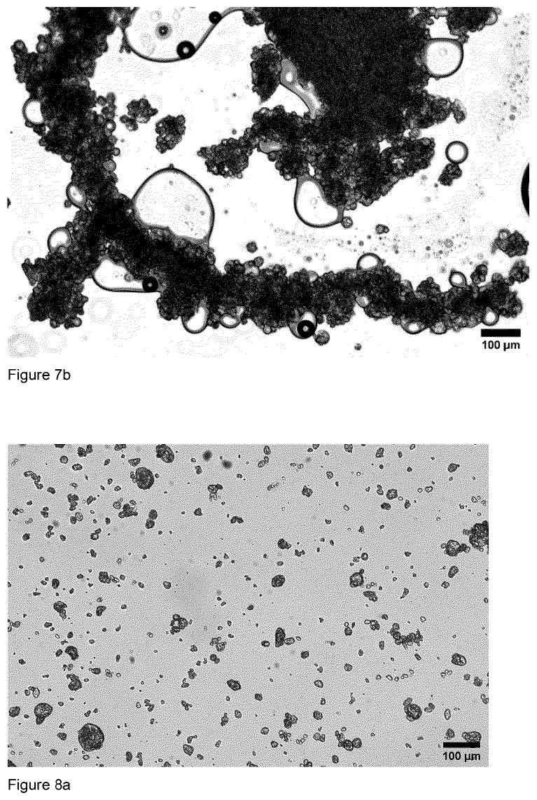

The microcapsules of Example 7 were rehydrated in hard water and optical microscope photographs taken (FIG. 7a). The same capsules were then pressed so that they broke to release the oil within (FIG. 7b). The oil droplets are clearly visible surrounding the broken microcapsules, with a very well-defined dark interface between the oil and water.

The microcapsules of Example 7 were analysed by the method described herein for their total fragrance loading. This was found to be 28.8%.

### Example 8—Preparation of Spray Dried Fragrance Microcapsules

### Preparation of Fragrance/Solvent Mixture

A mixture was prepared of delta-damascone and Miglyol® 812 in the volume ratio 80:20 by gentle mixing.

### Preparation of Spray Dried Microcapsules

The protein hydrogel slurry was made as per Example 1. The hydrogel slurry was diluted with acetic acid to a mean protein solids content of 8.0 wt %. A 221 g batch was stirred with an UltraTurrax lab mixer at 8000 rpm for 2 minutes. 33.1 g of the fragrance: oil mixture containing 80% delta-damascone and 20% Miglyol® 812 oil was then added. The composition is given in Table 6 below.

TABLE 6

| Material (wt %) | Example 8 |
|---|---|
| Pea Protein | 7.0 |
| Delta-damascone | 10.4 |
| Miglyol ® 812N | 2.6 |
| Water (acetic acid content approx. 3%) | 80.0 |
| Ratio PPI:Oil phase (fragrance + Miglyol) | 1:1.87 |

A fragrance emulsion was prepared as per Table 6, using hand shaking instead of high-shear mixing to form the emulsion. It was expected that the droplet size was around 20 microns

The sample was then spray-dried using a ProCepT R&D Spray Dryer in its three-column set-up. This gave a column height of 1.8 m with a column diameter of 0.15 m. The air inlet temperature was 120° C. at a flowrate of 400 l/min. The two-fluid nozzle was used with a tip size of 1.0 mm. The atomisation air flow rate was 10 l/min. The fluid was pumped into the spray dryer with a syringe pump at ml/min. An additional air flow of 1450 l/min was introduced below the drying chamber to convey air to the cyclone.

The dried microcapsule powder formed was collected from the cyclone and also from the internal surfaces of the equipment where it had stuck, for example in the transfer pipe to the cyclone.

The microcapsules of Example 8 were analysed by the method described herein for their total fragrance loading. This was found to be 18.7%.

### Example 9—Preparation of Spray Dried Fragrance Microcapsules

A fragrance composition was prepared as per Example 7 as in Table 5, however the emulsion was formed by hand shaking, instead of high-shear mixing with an Ultra Turrax. It was expected that the droplet size was around 20 microns.

The sample was then spray-dried using a ProCepT R&D Spray Dryer in its three-column set-up. This gave a column height of 1.8 m with a column diameter of 0.15 m. The air inlet temperature was 120° C. at a flowrate of 400 l/min. The two-fluid nozzle was used with a tip size of 1.0 mm. The atomisation air flow rate was 6.5 l/min. The fluid was pumped into the spray dryer with a syringe pump at 2 ml/min. An additional air flow of 150 l/min was introduced below the drying chamber to convey air to the cyclone.

The dried microcapsule powder formed was collected from the cyclone and also from the internal surfaces of the equipment where it had stuck, for example in the transfer pipe to the cyclone.

The microcapsules of Example 9 were analysed by the method described herein for their total fragrance loading. This was found to be 22.9%.

### Example 10—Coating of Spray Dried Fragrance Microcapsules with Shellac

30 g of 25 wt % of shellac solution were diluted in 120 ml of deionised water, in a tall form 250 ml glass beaker. Mechanical agitation was started at 350 rpm at room temperature using an anchor stirrer.

Separately hard water at pH3 was added drop by drop to 1.5 g of dry powder from Example 8 in a 50 ml Falcon tube, vortexing in between drop additions, in order to obtain a slurry paste. When the paste was homogeneous, further hard water at pH3 was added, still using vortexing to ensure a homogeneous dispersion of the particles. The microencapsulate slurry was further adjusted to a 50% (v/v) slurry in hard water at pH3.

The diluted microencapsulates were added dropwise to the stirring shellac solution, using a 3 ml transfer pipette. The mixture was left stirring for 30 minutes and then stored in the fridge for 3 days to allow the coating to form.

The crosslinked microcapsules were then subjected to the washing process whereby they were suspended in a separating funnel and sieved through a 250 μm sieve stacked with a 38 μm one to eliminate any possible aggregate resulting from the crosslinking step. The capsules collected on top of the 38 μm sieve were resuspended in a clean separating funnel in hard water to carry out an additional washing step. The decanted capsules were strained through a 40 μm cell strainer for 5 minutes. Any extra moisture was absorbed with a paper tissue. The strained capsules were diluted with single strength buffer to make a 50 wt % slurry.

The coated microcapsules of Example 10 were analysed by the method described herein for their total fragrance loading. This was found to be 0.8%.

### Example 11—Cross-Linking Spray Dried Fragrance Microcapsules with Tannic Acid and Complex Coacervation with Xanthan Gum

A 0.1 wt % xanthan gum solution was prepared under magnetic agitation in a water bath set at 60° C. until complete dissolution was observed. The solution was stored in the fridge overnight to allow for the gum to fully hydrate. The next day, the xanthan gum solution was adjusted to pH 3 using 1M HCl.

A 10 wt % tannic acid solution was prepared by hand shaking and vortexing for a few seconds using a Fisherbrand™ ZX4 IR Vortex Mixer, followed with immersion in an ultrasonic bath set at 50° C. for 5-10 min, until complete dissolution.

Separately hard water at pH3 was added drop by drop to 1.5 g of dry powder from Example 8 in a 50 ml Falcon tube, vortexing in between drop additions, in order to obtain a slurry paste. When the paste was homogeneous, further hard water at pH3 was added, still using vortexing to ensure a homogeneous dispersion of the particles. The microencapsulate slurry was further diluted in a total of 380 ml of hard water and adjusted to pH 3 using 1M HCl.

20.2 g of the tannic acid solution was added to the microencapsulate suspension under mechanical agitation at 170 rpm at 50° C., and left stirring for 5.5 hours to allow cross-linking to take place. After this the suspension was stored in the fridge overnight. The crosslinked microcapsules were then subjected to the washing process as in Example 10, resulting in brown coloured encapsulates indicating that crosslinking had occurred.

The cross-linked microencapsulates were then strained and suspended in 400 ml of hard water and adjusted to pH 3 using 1M HCl. The suspension was put under mechanical agitation at 350 rpm at room temperature. 545.7 g of the xanthan gum solution were slowly added while mixing and left to coacervate for 1.5 hour.

The final microcapsules were then subjected to the washing process as outlined in Example 10.

The microcapsules of Example 11 were analysed by the method described herein for their total fragrance loading. This was found to be 1.5%.

### Example 12—Coating Spray Dried Fragrance Microcapsules with Silica by Direct Immersion in TEOS

300 mg of dry powder from Example 9 was directly added to 2 g of TEOS and mixed by hand to form a suspension. The microencapsulate suspension was mixed at 200 rpm for 6 hours ensuring the capsules do not sediment during the reaction. The microcapsules were then sedimented by centrifugation at 4500 rpm for 5 min and the majority of the supernatant was removed. Sedimented microcapsules were resuspended in the remaining TEOS and pipetted onto filter paper to air-dry overnight and remove excess TEOS.

The microcapsules of Example 12 were analysed by the method described herein for their total fragrance loading. This was found to be 6.3%.

### Example 13—Preparation of Protein Hydrogel Slurry with Acetic Acid

### Preparation of the Protein Hydrogel

1120 g of reverse osmosis (RO) water was added to a 2-litre stainless steel container, and 216 g of pea protein isolate was added. The container was placed in a 92° C. water bath and mixed with an overhead stirrer at 1500 rpm. After stirring for 3 minutes, 480 g glacial acetic acid was added. The mixture was stirred for 15 minutes at 1500 rpm, then for 30 minutes at 1200 rpm, ensuring that the temperature of the mix surpassed 85° C. for at least 10 minutes. The mixture was poured into trays to a depth of approximately 10 mm, and left at room temperature overnight.

### Application of Shear to Protein Hydrogel

Shear was then applied to the hydrogel as follows. The protein hydrogel was cut into ˜1 cm cubes via a low-shear cutting step. The cubes were split between two 75 micron filter bags, which were each then submerged inside a bucket containing 16 L of RO water. This formed a coarse protein hydrogel slurry within the filter bag. The hydrogel cubes were left to soak, with agitation from an overhead stirrer at 600-800 rpm, for 90-150 min. This step was performed to reduce the concentration of acetic acid in the hydrogel by diffusion to the continuous aqueous phase. The pH of the wash water was then measured, and if it was above 3.2, soaking was continued for a further 30 minutes. If it was below 2.9, half of the water was drained and replaced with fresh RO water, then soaking was continued for a further 30 minutes. The filter bag was then suspended above the bucket to drain for 5 minutes. The washed gel from both filter bags was transferred to a 5 liter beaker, and homogenized with a Silverson mixer at 5000 rpm for 5 minutes, 6000 rpm for 5 minutes, and 7000 rpm for 5 minutes. The smooth slurry was then transferred to 1 L Nalgene bottles (800 g in each), and exposed to high shear ultrasonication (Hielscher UP500Hdt) with cooling on ice, until 250 kJ had been applied, with shaking every 75 kJ. The hydrogel slurry was then passed through a 200 micron sieve before use.

### Comparative Example 14—Preparation of Protein Slurry without Acid

450 g of reverse osmosis water was added to a 1 L stainless steel container, and 50 g of pea protein isolate was added. The container was placed in a 92° C. water batch and mixed with an overhead stirrer at 1500 rpm. The mixture was stirred for 15 minutes at 1500 rpm, then for 30 minutes at 1200 rpm, ensuring that the temperature of the mix surpassed 85° C. for at least 10 minutes. On cooling the mixture to ambient temperature, in contrast to the experiments with acetic acid, a gel was not formed. Shear was then applied with a Silverson mixer at 8000 rpm for 10 minutes, then with an ultrasonicator (Hielscher UP500Hdt) until 156 kJ had been applied. For comparison 0.3125 KJ/g was used for protein slurries prepared with organic acid. The mixture was then passed through a 200 micron sieve before use.

### Example 15—Preparation of Spray-Dried Microcapsules Containing Vitamin D2

### Preparation of Composition

Vitamin D2 oil (1 MIU/g) was diluted in Softisan® 100 wax to a concentration of 6000 ug/g. This concentration was chosen to target the manufacture of spray dried particles containing a Vitamin D2 concentration of 1,000 ug/g. The wax was melted above 40° C., the vitamin oil added, and the mixture homogenised by hand shaking. The mixture was held above 40° C. until required.

The required mass of wax and Vitamin D2 mixture was then added to the protein hydrogel slurry of Example 13 and mixed with a Silverson L5M-A homogenizer at 7000 rpm for 10 minutes to form a composition with 11.9 weight % total solids including vitamin D2 and wax. The composition was then further diluted with 3.0% aqueous acetic acid to produce 2 samples: Example 15A with a total solids content of 10.0% including the vitamin D2 and wax (8.9 weight % protein solids) and Example 15B with a total solids content of 5.0% including the vitamin D2 and wax (4.4 weight % protein solids).

The required mass of wax and Vitamin D2 mixture was also added to the protein slurry of Comparative Example 14 and mixed with a Silverson L5M-A homogenizer at 7000 rpm for 10 minutes to form a composition. The composition was then further diluted with 3.0% aqueous acetic acid to produce Comparative Example 15C with a total solids content of 11.7 weight % including the Vitamin D2 and wax (10 weight % protein solids).

### Spray Drying Conditions

For Examples 15A and 15B the spray drying was performed using a ProCept spray dryer in its three-column set-up. This gave a column height of 1.8 m with a column diameter of 0.15 m.

For Example 15A the two-fluid nozzle was used with a tip size of 1 mm. The atomisation air flow rate was 10 l/min. The fluid was pumped into the spray dryer with a syringe pump at 6 ml/min. An additional air flow of 150 l/min was set for the cyclone.

For Example 15B the ultrasonic nozzle was used. The atomisation air flow rate was 10 l/min. The fluid was pumped into the spray dryer with a syringe pump at 6 ml/min. An additional air flow of 150 l/min was set for the cyclone.

For comparative Example 15C the spray drying was performed using Buchi B290 Spray Dryer with a two-fluid nozzle was used with a tip size of 1.4 mm. The aspirator flow rate was set at 100%, with a Q-flow setting of 40. The fluid was pumped into the spray dryer with a peristaltic pump at a speed setting of 35% (approx. 10 ml/min). The dried microcapsule powder formed was collected from the collection pot.

Other process settings are given in Table 7 below.

TABLE 7

| Microcapsule | Inlet air flow | Inlet air temp | Chamber out | Air out temp |
|---|---|---|---|---|
| Example | rate (m³/min) | (° C.) | temp (° C.) | (° C.) |
| Example 15A | 0.4 | 140 | 79.6 | 63.1 |
| Example 15B | 0.4 | 140 | 82.6 | 71.0 |
| Comparative | 0.58 | 180 | 110-114 | n/a |
| Example 15C | | | | |

The spray drying experiments produced three free flowing powders with a mean particle size, d₅₀, measured by laser diffraction according to the method therein, of 28.9 microns, 40.8 microns and 9.1 microns for Examples 15A, 15B and Comparative Example 15C respectively. It is noted that for the acid treated plant protein encapsulate the more diluted composition dried with an ultrasonic nozzle produced larger microcapsules. The non-acid treated plant protein with a similar solids content the powder had a much smaller average particle size.

The Vitamin D2 concentration in the microcapsule powder was measured using the method Version B described herein and was found to be 963 μg/g, 919 μg/g and 1149 μg/g in Examples 15A, 15B and Comparative Example 15C, respectively. This was close to the expected target of 1000 μg/g of microcapsule indicating very little loss of Vitamin D2 during the spray drying process.

### Example 16—Accelerated Stability Testing of Vitamin D2 Microcapsules in a Beverage Formulation

### Preparation of Beverage Samples

The microcapsules of Examples 15A, 15B and Comparative Example 15C were each prepared into a concentrated slurry so that they could then be added to Miwadi Blackcurrant single concentrate squash.

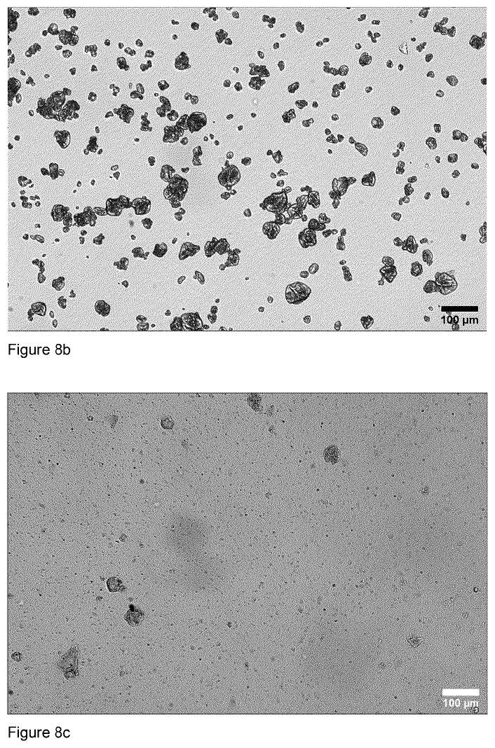

0.2 g of each microcapsule was first suspended in 30 g of boiling water with vigorous shaking, followed by vortexing at 2000 rpm for 1 minute to mimic the temperature profile during flash pasteurization. It was noted that the concentrated slurry prepared with Comparative Example 15C was more hazy and appeared finer than Examples 15A and 15B. FIGS. 8a, 8b and 8c are 10× microscope images of the concentrated slurry of Example 15A, 15B and Comparative Example 15C. Example 15A and 15B microcapsules can be seen and have clearly survived the processing. For Comparative Example 15C the image is notable for the lack of intact microcapsules demonstrating that subjecting them to heat and shear caused the microcapsules to quickly disintegrate. In their place smaller aggregates of proteins and very small particles of wax and Vitamin D2 can be seen.

### Accelerated Stability Testing

A calculated mass of each concentrated slurry was added to 2 liters of squash and mixed with an overhead mixer at 800 rpm for 5 minutes to ensure homogeneity before transferring to bottles.

200 ml transparent plastic PET bottles were filled with each of the three squash samples and initial Vitamin D2 loading was measured using method A herein described. The initial beverage Vitamin D loading of Examples 15A and 15B are given in Table 7. However, no initial measured beverage loading of Vitamin D2 in Comparative Example 15C could not be detected. Without wishing to be bound by theory it is believed that this is because in the concentrated slurry preparation, where the microcapsules were suspended and heated, they were broken so releasing the Vitamin D2. The Vitamin D2 oil floated to the top of the bottle and was not sampled.

Filled PET bottles of Examples 15A and 15B were then placed in a Binder KBF P 240-230 V chamber, which was set for accelerated ambient light stability studies, in its standard configuration with visible and UV-A tubes. The nominal UV-A output was 1.1 W/m². The temperature was set to 20° C. and lab humidity was used (typically 50% relative humidity (RH)). The Vitamin D loading was again measured, using the method A herein described, after 4 weeks in the Binder chamber. The % reduction in Vitamin D2 content was calculated and recorded in Table 8.

TABLE 8

| | Initial Beverage | | |
|---|---|---|---|
| | Vitamin D2 | Microcapsule | % Vitamin D2 |
| | Loading, | Particle Size, d 50 | loss after 4 |
| Microcapsule | μg/100 g | (microns) | weeks |
| Example 15A | 3.0 | 28.9 | 22.5% |
| Example 15B | 2.0 | 40.8 | 5.5% |

After 4 weeks in the squash the Vitamin D2 in the encapsulate of the invention had been protected from degradation so that for Example 15A only 22.5% of the initial Vitamin D2 had been lost and for Example 15B only 5.5% of the initial Vitamin D2 had been lost. Unencapsulated vitamin D2 is expected to degrade significantly on exposure to UV over this time frame, but this low level of loss demonstrated the protection provided by the inventive microcapsule. It can also be seen that the lowest loss was achieved with the larger average encapsulate size. Without wishing to be bound by theory, it is believed that the larger the average particle size of the encapsulate the lower the surface area available per mass of Vitamin D2 for exposure to UV and therefore the less the degradation.

### Example 17—Encapsulation of Natural Agrochemical Actives

### Example 17A: Thymol Protein Microcapsule Preparation

### Preparation and Application of Shear to the Protein Hydrogel

The protein hydrogel shell was prepared and sheared as described in Example 13.

The protein solid content was measured at 9.3 wt %. Dilute acetic acid (3 wt % in DI water) was added to reduce the protein solids content to 8.0%.

### Preparation of Spray-Dried Microcapsule

The diluted dispersion was homogenised with a Silverson L5M-A high-shear mixer (2 minutes at 8000 rpm). 16.81 g of thymol, diluted at 40 wt % in Miglyol 812N, was added to 103.25 g of diluted dispersion. The mixture was homogenised further with the Silverson at 8000 RPM for 5 minutes. It was expected that the droplet size was around 5 microns.

The sample was then spray-dried using a Buchi B290 Spray Dryer. The air inlet temperature was 120° C. at Q-flow setting of 40. The two-fluid nozzle was used with a tip size of 1.4 mm. The aspirator flow rate was set at 100%. The fluid was pumped into the spray dryer with a peristaltic pump at a speed setting of 13% (4-5 ml/min).

The dried microcapsule powder formed was collected from the collection pot. The microcapsules were analysed for their total thymol loading. This was found to be 4.9 wt %. The particle size was measured via laser diffraction and found to have a d50 of 23 microns.

### Example 17B: Comparative Maltodextrin Thymol Microcapsule Preparation

45 g maltodextrin was dissolved in 105 g of deionised water and mixed with a magnetic stirrer bar until a homogenous slurry was formed. 15.23 g of thymol, diluted at 40 wt % in Miglyol 812N, was added to the slurry, in addition to 0.14 g of polysorbate 80. The mixture was homogenised with a Silverson L5M-A high-shear mixer for 5 minutes at 8000 rpm.

The sample was then spray-dried using a Buchi B290 Spray Dryer. The air inlet temperature was 130° C. at Q-flow setting of 40. The two-fluid nozzle was used with a tip size of 1.4 mm. The aspirator flow rate was set at 100%. The fluid was pumped into the spray dryer with a peristaltic pump at a speed setting of 13% (4-5 ml/min).

The dried microcapsule powder formed was collected from the collection pot. The microcapsules were analysed for their total thymol loading. This was found to be 3.4 wt %. It was not possible to measure the particle size of these microcapsules as they were too fragile. When placed on a microscope slide with a cover slip it could be seen that the weight of the cover was sufficient to break them.

### Example 17C: Aqueous Release Test

The thymol microcapsule powder of Example 12A was suspended in phosphate buffered saline (PBS) (pH 7.4) containing 0.2% w/v methylparaben and a solution of sodium acetate and calcium chloride according to Table 9, such that the sample contained 4 mg of thymol. Replicate samples were prepared so that samples could be analysed at t=0, 24 hours, 72 hours, 1 week, and 2 weeks of incubation.

TABLE 9 - Composition of test sample

| | Mass of | Mass of 10 mM | Mass of PBS pH |
|---|---|---|---|
| | microcapsule | NaOAc + 5 mM | 7.4, +0.2% wt |
| Test sample | powder (mg) | CaCl2 (mg) | methylparaben (mg) |
| Example 17A | 82 | 459 | 3541 |

The samples were mixed on day 0 and placed in a 37° C. incubator in the dark, except for t=0 samples, which were analysed immediately. At each time point, samples were centrifuged for 30 min at 4900 RPM, and the supernatant transferred to 50 ml Falcon tubes. Ethanol was added to achieve a tenfold dilution of the supernatant samples. The concentration of thymol in the supernatant was measured by gas chromatography, which was used to calculate the quantity of thymol released from the microcapsules.

To the pelleted material after centrifugation of each sample, 2.9 ml of DI water and 0.1 ml of 10% KOH solution were added. The mixtures were ultrasonicated (Bandelin Sonopuls HD4200, with probe TS104) for 2 minutes at 30% amplitude, vortexed and centrifuged for 5 minutes at 4900 rpm. 100 μl of the supernatant from each sample was diluted tenfold with ethanol, then the concentration of thymol measured by gas chromatography. This was used to calculate the quantity of thymol remaining in the sample at the end of the experiment, which was taken to be the quantity of thymol not originally released in the first supernatants. The results are shown in Table 10.

TABLE 10

| Microcapsule | % extractable thymol in | % extractable thymol in |
|---|---|---|
| Example 17A | supernatant | remaining shell material |
| t = 0 | 3 | 97 |
| t = 24 hr | 5 | 95 |
| t = 72 hr | 5 | 95 |
| t = 1 week | 8 | 92 |
| t = 2 weeks | 6 | 94 |

This release study demonstrated that most of the active ingredient, more than 90%, remained encapsulated over 2 weeks. Such a slurry would be suitable for storage prior to being applied to fields and crops.

### Example 17D: Comparative Maltodextrin-Thymol Release Test

The thymol microcapsule powder of Comparative Example 17B was suspended in phosphate buffered saline (PBS) (pH 7.4) containing 0.2% w/v methylparaben according to Table 11, such that the sample contained 4 mg of thymol, the same level as in Example 17A. There was only a minor difference in the suspension composition with the absence of the low levels of NaOAc and CaCl2 which is of no consequence in this example.

TABLE 11

| | Mass of microcapsule | Mass of PBS pH 7.4, +0.2% |
|---|---|---|
| Test sample | powder (mg) | wt methylparaben (mg) |
| Comparative Example 17B | 119 | 4000 |
| Maltodextrin microcapsules | | |

The sample was prepared, mixed and incubated for one hour. The sample was centrifuged for 10 min at 4900 RPM, and the supernatant transferred to 50 ml Falcon tube. Ethanol was added to achieve a tenfold dilution of the supernatant samples. The concentration of thymol in the supernatant was measured by gas chromatography, which was used to calculate the quantity of thymol released from the capsules in buffered water.

To the pelleted material after centrifugation of sample, 2.9 ml of deionised water and 0.1 ml of 10% KOH solution were added. The mixture was ultrasonicated (Bandelin Sonopuls HD4200, with probe TS104) for 2 minutes at 30% amplitude, vortexed and centrifuged for 5 minutes at 4900 rpm. 100 μl of supernatant was diluted tenfold with ethanol, then the concentration of thymol measured by gas chromatography. This was used to calculate the quantity of thymol remaining in the sample at the end of the experiment, which was taken to be the quantity of thymol not originally released in the first supernatant. The results are shown in Table 12.

TABLE 12

| | % extractable | |
|---|---|---|
| Comparative Example 17B | thymol in | % extractable thymol in |
| Maltodextrin microcapsules | supernatant | remaining shell material |
| t = 1 hour | 42 | 58 |

This release study demonstrated that for maltodextrin microcapsules, in Comparative Example 17B, a substantial portion of active ingredient, more than 40%, was released after 1 hour in an aqueous buffer. Such a slurry would not be suitable for storage prior to being applied to fields and crops. Once prepared into an aqueous composition, or in the presence of water in the environment such as rain, it would prematurely release the active ingredient thereby demonstrating no controlled release properties.

### Example 18—Preparation of Spray-Dried Microcapsules Containing Fragrance Oils

### Fragrance/Solvent Mixtures

Two different fragrances (18A and 18B) were prepared according to Table 13. Each fragrance was then blended with one of two solvents: either Miglyol® 812N or isopropyl myristate (IPM) in a weight ratio of 80:20 fragrance: solvent.

Fragrance 18A comprises only moderately volatile fragrance materials of similar volatility with a range of hydrophobicity from very low to very high. Fragrance 18B comprises materials of similar hydrophobicity with the full range of volatilities including low, moderate and high volatility materials. Both fragrances had fragrance materials with HSP parameters in the range δD from 15 to 20, δP from 1 to 7 and δH from 2.5 to 8.

TABLE 13

| | | Vapour | | | | | | Fragrance | Fragrance |
|---|---|---|---|---|---|---|---|---|---|
| Fragrance | CAS | pressure | | HSP | | | Function- | 18A | 18B |
| material | number | (Torr) | LogP | δD | δP | δH | ality | (wt %) | (wt %) |
| Dipentene | 138-86-3 | 1.541 | 4.40 | 17.2 | 1.8 | 4.3 | Alkene | n/a | 20 |
| Ortho | 88-41-5 | 0.103 | 4.46 | 15.9 | 1.9 | 2.9 | Ester | n/a | 20 |
| tertiary | | | | | | | | | |
| butyl | | | | | | | | | |
| cyclohexyl | | | | | | | | | |
| acetate | | | | | | | | | |
| (OTBCHA) | | | | | | | | | |
| Diphenyl | 101-84-8 | 0.022 | 4.03 | 19.4 | 3.4 | 4.0 | Ether | n/a | 20 |
| oxide | | | | | | | | | |
| Geraniol | 106-24-1 | 0.013 | 3.42 | 16.9 | 4.2 | 7.6 | Primary | 25 | n/a |
| | | | | | | | alcohol | | |
| Undecavertol | 81782-77-6 | 0.011 | 3.97 | 16.4 | 3.4 | 7.5 | Secondary | 25 | n/a |
| | | | | | | | alcohol | | |
| Delta | 57378-68-4 | 0.010 | 3.56 | 16.9 | 3.5 | 2.6 | Ketone | 25 | 20 |
| damascone | | | | | | | | | |
| dodecane | 2437-25-4 | 0.005 | 5.12 | 16.0 | 5.4 | 3.1 | Nitrile | 25 | n/a |
| nitrile | | | | | | | | | |
| Benzyl | 118-58-1 | 0.0002 | 4.09 | 19.3 | 6.5 | 7.8 | Ester | n/a | 20 |
| salicylate | | | | | | | | | |

### Preparation of Spray Dried Microcapsules

The protein hydrogel slurry was made as per Example 13.

A first batch of fragrance composition was prepared as follows. A batch of protein hydrogel slurry of Example 13 of between 100 and 120 g was diluted by 20% with 3% acetic acid solution to give a slurry with a protein solids content of 8.0 wt %. The diluted slurry was then stirred with a Silverson lab mixer at 8000 rpm for 2 minutes in a 400 ml Nalgene beaker. 31.5 wt % (based on the weight of the slurry) of Fragrance 18B/Miglyol® 812N blend was then added to the slurry and emulsified using a Silverson mixer at 8000 rpm for a further 5 mins to form a fine emulsion. This fine emulsion was left to age at room temperature for approximately 2 days prior to spray drying. This mix was used in Run 2 TTB.

A second batch of fragrance composition was prepared as above except that the fragrance blend was emulsified just prior to spray-drying by gentle hand shaking rather than by use of the Silverson mixer. This mix was used in Run 3 TTB.

A third batch of fragrance composition was prepared as the first batch except that the Miglyol® 812N was replaced by isopropyl myristate (IPM) to form the fragrance: solvent blend. The fragrance and slurry mixture was emulsified by using a Silverson mixer at 8000 rpm for a further 5 mins to form a fine emulsion. This fine emulsion was left to age at room temperature for approximately 2 days prior to spray drying. This mix was used in Run 6 TTB.

A fourth batch of fragrance composition was prepared with the same composition and in the same manner as the material used in Run 6 TTB and used in Run 7 CPB and Run 8 TTB.

The samples were spray-dried using a ProCepT R&D Spray Dryer in its three-column set-up. This gave a column height of 1.8 m with a column diameter of 0.15 m.

The air inlet temperature was varied to achieve a dry powder. It was kept as low as possible to reduce the loss of volatile fragrance materials. The drying inlet air flow rate was kept constant at 0.4 m³/min. The two-fluid nozzle from Spray Systems was used with a tip size of 1.0 mm. The atomisation air flow rate was varied between runs as shown in Table 14 below. The fluid was pumped into the spray dryer with a syringe pump at 4 ml/min for all the runs. An additional air flow of 150 l/min was introduced below the drying chamber to convey air to the cyclone.

The dried microcapsule powder formed was collected in the collection pot and also from the internal surfaces of the equipment where it had stuck, for example in the transfer pipe to the cyclone. The microcapsules particle size was measured by laser diffraction.

Fragrance 18B was successfully encapsulated as given in Table 14.

TABLE 14

| Fragrance | | | | | |
|---|---|---|---|---|---|
| composition 18B | Run2TTB | Run3TTB | Run6TTB | Run8TTB | Run7CPB |
| Core | Miglyol ® | Miglyol ® | IPM | IPM | IPM |
| | 812N | 812N | | | |
| Emulsion type | Fine | Coarse | Fine | Fine | Fine |
| | aged | fresh | Aged | Aged | Aged |
| Atomisation | 15 | 15 | 15 | 12 | 8 |
| (ltr/min) | | | | | |
| Air inlet ° C. | 68.6-68.2 | 68.5-68.7 | 68.5-68.8 | 80.1-88.4 | 82.3-83.5 |
| Air outlet ° C. | 43.5-41.5 | 41.6-43.7 | 42.1-54.7 | 48.4-61.5 | 40.3-42.6 |
| Particle size, d50 | 13.3 | 30.1 | 12.2 | 14.6 | 40.1 |
| (microns) | | | | | |

When drying at a lower temperature (inlet around 69° C.) the largest particle size of 30.1 microns, was produced with the coarse fresh emulsion with Miglyol® 812N as the solvent, prepared just before spray drying. Changing the fragrance solvent from Miglyol® 812N to isopropyl myristate within the fine aged emulsion had very little effect on the particle size.

Reducing the atomising air flowrate resulted in larger particles (compare Run 8TT with Run 7CP).

The fragrance material composition of these microcapsules was analysed using the fragrance loading quantification method via GC described herein. Results are given in Table 15.

TABLE 15

| Fragrance | | | | | |
|---|---|---|---|---|---|
| composition 18B | Run2TTB | Run3TTB | Run6TTB | Run8TTB | Run7CPB |
| Core | Miglyol ® | Miglyol ® | IPM | IPM | IPM |
| | 812N | 812N | | | |
| Emulsion type | Fine | Coarse | Fine | Fine | Fine |
| | aged | fresh | Aged | Aged | Aged |
| Total fragrance | 45.4 | 51.3 | 48.3 | 41.5 | 44.4 |
| oil loading wt % | | | | | |
| Wt % Dipentene | 0.6 | 1.5 | 0.7 | 0.6 | 0.7 |
| Dipentene oil % | 1.3 | 2.9 | 1.4 | 1.4 | 1.6 |
| Wt % | 5.0 | 6.3 | 5.5 | 4.3 | 5.1 |
| (OTBCHA) | | | | | |
| (OTBCHA) oil | 11.0 | 12.2 | 11.4 | 10.4 | 11.5 |
| % | | | | | |
| Wt % Diphenyl | 11.4 | 11.8 | 11.6 | 9.0 | 10.5 |
| ether | | | | | |
| Diphenyl ether | 25.1 | 23.0 | 24.0 | 21.7 | 23.6 |
| % | | | | | |
| Wt % Delta | 6.4 | 6.8 | 5.8 | 4.5 | 5.7 |
| Damascone | | | | | |
| Delta damascone | 14.1 | 13.3 | 12.0 | 10.8 | 12.8 |
| oil % | | | | | |
| Wt % Benzyl | 22.0 | 24.9 | 24.7 | 23.1 | 22.5 |
| salicylate | | | | | |
| Benzyl salicylate | 48.4 | 48.5 | 51.1 | 56.3 | 49.5 |
| oil % | | | | | |

Samples dried at lower air inlet temperatures typically had a higher total fragrance loading than those dried at higher air inlet temperatures.

When drying at the lower temperature (inlet around 69° C.), isopropyl myristate resulted in higher total loading than Miglyol®812N for a fine emulsion. In addition, at these drying conditions the coarse emulsion resulted in higher loading.

When drying at a higher temperature (inlet at least 80° C.) with the atomisation air flowrate reduced a higher total loading was achieved than with high air atomisation.

Fragrance 18B demonstrated that for fragrance materials of similar hydrophobicity it is possible to encapsulate materials with a range of volatilities. However as expected the least volatile fragrance materials are enriched in the encapsulate with benzyl salicylate accounting for between 48.4 and 56.3% of the fragrance whereas it was 20% of the fragrance before encapsulation. Drying at lower temperatures and with a coarse emulsion improved the encapsulation of the more volatile fragrance materials such as Limonene from 0.6% to 1.5% of the fragrance.

Fragrance Composition 18A was also successfully encapsulated as given in Table 16.

A first batch of fragrance composition was prepared as follows. A batch of protein hydrogel slurry of Example 13 of between 100 g and 120 g was diluted by 20 wt % with 3% acetic acid solution to give a slurry with a protein solids content of 8.0 wt %. The diluted slurry was then stirred with a Silverson lab mixer at 8000 rpm for 2 minutes in a 400 ml Nalgene beaker. 28.3 wt % (based on the weight of the slurry) of Composition 18A/Miglyol® 812N blend was then added to the slurry and emulsified using a Silverson mixer at 8000 rpm for a further 5 mins to form a fine emulsion. This fine emulsion was left to age at room temperature for approximately 2 days prior to spray drying. This mix was used in Run 1 TTA.

A second batch of fragrance composition was prepared as above. Immediately prior to use, 120 g of the emulsion was diluted with 60 g of 3% acetic acid solution with gentle agitation. This mix was used in Run 9 TTA.

A third batch of fragrance composition was prepared in the same manner as the first batch above except that the fragrance blend was sheared and emulsified just prior to spray-drying rather than being allowed to age. This mix was used in Run 3 TTA.

A fourth batch of fragrance composition was prepared as above except that the fragrance blend was emulsified just prior to spray-drying by gentle hand shaking rather than by use of the Silverson mixer. This mix was used in Run 2 TTA.

A fifth batch of fragrance composition was prepared in the same manner as the first batch but now with the Miglyol®812N replaced by isopropyl myristate (IPM). This mix was used in Run 4TTA.

A sixth batch of fragrance composition was prepared in the same manner as the fifth batch and this mix was used in Run 5CPA.

The same equipment was used as in the earlier runs with drying air flowrates, emulsion flow rates and transfer air rates the same as for Fragrance Composition 18B. The dried microcapsule powder formed was again collected in the collection pot and also from the internal surfaces of the equipment where it had stuck, for example in the transfer pipe to the cyclone. The microcapsules particle size was measured by laser diffraction.

TABLE 16

| | | | | | | |
|---|---|---|---|---|---|---|
| Fragrance | Run1TTA | Run9TTA | Run3TTA | Run2TTA | Run4TTA | Run5CPA |
| composition 18A | | | | | | |
| Core | Miglyol ®812N | Miglyol ®812N | Miglyol ®812N | Miglyol ®812N | IPM | IPM |
| Emulsion type | Fine | Diluted | Fine | Coarse | Fine | Fine |
| | aged | Fine | fresh | fresh | aged | aged |
| | | aged | | | | |
| Atomisation air | 15 | 10 | 15 | 15 | 15 | 10 |
| flow (ltr/min) | | | | | | |
| Air inlet | 69.7-69.1 | 68.8-63.7 | 68.7-68.5 | 68.9-68.8 | 68.8-68.5 | 83.5-68.7 |
| temperature ° C. | | | | | | |
| Air outlet | 50.2-47.6 | 43.4-40.8 | 43.4-41.1 | 37.7-37.0 | 44.0-42.1 | 63.2-46.5 |
| (transfer tube) | | | | | | |
| temperature ° C. | | | | | | |
| Particle size, | 12.9 | 15.3 | 29.9 | 53.8 | 16.4 | 22.3 |
| d50 | | | | | | |

The largest particle size was produced with the coarse fresh emulsion with Miglyol®812N as the fragrance solvent prepared just before spray drying.

The fine aged emulsion, with Miglyol®812N as the fragrance solvent, resulted in the smallest microencapsulate particle size. Dilution of the fine aged emulsion and running at lower drying temperatures did not affect the particle size. Changing the fragrance solvent from Miglyol®812N to isopropyl myristate within the fine aged emulsion also had very little effect on the particle size.

A fine emulsion spray dried after ageing had a smaller particle size than the same freshly made emulsion.

The fragrance material composition of these microcapsules was analysed using the fragrance loading quantification method via GC described herein. Results are given in Table 17.

TABLE 17

| | | | | | | |
|---|---|---|---|---|---|---|
| Fragrance | Run1TTA | Run9TTA | Run3TTA | Run2TTA | Run4TTA | Run5CPA |
| composition 18A | | | | | | |
| Core | Miglyol812 | Miglyol812 | Miglyol812 | Miglyol812 | IPM | IPM |
| Emulsion | Fine | Fine aged - | Fine | Coarse | Fine | Fine |
| type | aged | diluted | fresh | fresh | aged | aged |
| Total | 29.1 | 24.6 | 27.8 | 26.7 | 26.3 | 30.4 |
| fragrance | | | | | | |
| oil loading | | | | | | |
| wt % | | | | | | |
| wt % | 6.7 | 6.3 | 6.9 | 6.6 | 6.3 | 7.1 |
| Geraniol | | | | | | |
| Geraniol oil | 21.3 | 25.6 | 25.1 | 24.7 | 24.0 | 23.3 |
| % | | | | | | |
| wt % | 5.0 | 5.1 | 5.5 | 5.5 | 5.1 | 5.6 |
| Undecavertol | | | | | | |
| Undecavertol | 17.2 | 20.7 | 19.8 | 20.6 | 19.4 | 18.4 |
| oil % | | | | | | |
| wt % Delta- | 4.4 | 3.2 | 4.0 | 4.3 | 3.6 | 4.3 |
| damascone | | | | | | |
| Delta | 15.1 | 13.0 | 14.4 | 16.1 | 13.7 | 14.1 |
| damascone | | | | | | |
| oil % | | | | | | |
| wt % | 13.1 | 10.0 | 11.5 | 10.4 | 11.3 | 13.4 |
| Dodecane | | | | | | |
| nitrile | | | | | | |
| Dodecane | 45.0 | 40.7 | 41.4 | 39.0 | 43.0 | 44.1 |
| nitrile oil % | | | | | | |

All samples had similar total fragrance loadings but the fine aged sample with IPM sprayed with the lower air atomisation rate had the highest loading at 30%. This is noticeably less than Fragrance Composition 18B which had a maximum total fragrance loading of 51%. Without wishing to be bound by theory this is likely due to the presence of very low log P fragrance materials in Fragrance 18A.

Fragrance Composition 18A contained 4 fragrance materials of similar moderate volatility and varying hydrophobicity and demonstrates that it is possible to encapsulate materials with a range of hydrophobicity. The composition of the encapsulated fragrance was richer in the most hydrophobic fragrance material, dodecane nitrile, for all the examples. This was least noticeable in the coarse emulsion sample.

Delta-damascone was the least enriched fragrance material despite having a higher log P than geraniol and undecavertol. These two alcohols were well encapsulated, particularly geraniol, demonstrating that it is still possible to encapsulate these relatively hydrophilic fragrance materials within a fragrance.

### Olfactive Evaluation of Spray Dried Microcapsules

A concentrated slurry of the microcapsule of Run 3TTA was prepared prior to making the diluted formulation for olfactive evaluation. Some single strength buffer solution with preservative was added drop by drop to the dry microcapsules in a 50 ml Falcon tube, vortexed at 2000 rpm in between drop additions, in order to obtain a slurry paste. When the paste appeared homogeneous, more buffer solution was added for further dilution, still using vortexing as a means of ensuring homogeneous dispersion of the microcapsules. The suspension was left to stand for 1h to allow for possible swelling of the microcapsules before being strained through a 40 μm cell strainer for 5 minutes. Any extra moisture was absorbed with paper tissue.

The total fragrance level was analysed by GC according to the method herein to be 12.6 wt %. This is lower than the total fragrance level of the spray dried microcapsules. Whilst not wishing to be bound by theory it is believed that as the hydrogel shell has been hydrated and swollen it has resulted in an increase in the mass of the shell. In addition, a small amount of surface fragrance may have been lost into the aqueous phase.

A diluted slurry of microcapsules containing gellan gum was prepared so that it contained 0.1 wt % total fragrance for olfactive evaluation. Gellan gum helps maintain the microcapsules well suspended. A solution of gellan gum was prepared by mixing 9.5 mg of gellan gum with 15 g of deionised water in a 50 ml Falcon tube. The mixture was heated to above 90° C. for 20 minutes and stirred at 500 rpm using an Eppendorf Thermomixer C set at 100° C. After cooling to room temperature, 15 g of double strength buffer solution was added. The concentrated encapsulate slurry was added to the gellan gum solution using a micropipette with a low shear tip, and the sample tube was closed and inverted repeatedly to mix.

The pH of the diluted slurry was 3.05. The odour of the diluted slurry was evaluated according to the method herein described, and the results are shown in Table 18.

TABLE 18

| Substrate | | Glass | Card |
|---|---|---|---|
| Initial | Strength | 3.75 | 4.25 |
| application | Character | Fruity (orange), ester, white | Fruity (orange), ester, |
| | | floral, green, slightly medicinal | medicinal, aldehydic, green |
| 24 hours | Strength | 0.5 | 0.7 |
| | Character | slightly fruity, harsh | Slightly floral, powdery |
| Rubbing | Strength | 4.4 | 3.0 |
| | Character | Sweet, ester, fruity, tangerine | Sharp fruity, aldehydic, fizzy |
| | | peel, green, harsh/chemical, | sherbet |
| | | aldehydic, medicinal | |
| Pressure | Strength | 4.2 | Not applicable |
| with a | Character | Sweet, ester, fruity, tangerine | Not applicable |
| slide | | peel, green, harsh/chemical, | |
| | | aldehydic, medicinal | |

On initial application there was a strong characteristic odour of the fragrance indicating that some of the fragrance oil is associated with the surface of the hydrogel shell. After 24 hours of drying the odour was very weak indicating that most of the surface fragrance had evaporated. On rubbing or pressing on glass a strong characteristic odour was noticed as the force ruptures that microcapsule releasing the fragrance. On rubbing on card the odour was of moderate strength and the fragrance character was somewhat different to that on glass, indicating that there was only partial release of the encapsulated fragrance.

### Example 19—Cross-Linking of Spray Dried Fragrance Microcapsules

Spray dried microcapsules were prepared as in Example 18 Run 3TTA.

Hard water adjusted to pH3 was added drop by drop to 0.8 g of dry microcapsules in a 50 ml Falcon tube, vortexed at 2000 rpm in between drop additions, to obtain a slurry paste. When the paste appeared homogeneous, more hard water at pH3 was added for further dilution, still using vortexing as a means of ensuring homogeneous dispersion of the microcapsules. The suspended microcapsules were further diluted in a total of 390 ml of hard water and the pH was adjusted to 3.0±0.5.

A 10 wt % tannic acid solution was prepared by hand shaking and vortexing for a few seconds using a Fisherbrand™ ZX4 IR Vortex Mixer, followed with immersion in an ultrasonic bath set at 50° C. for 5-10 min, until complete dissolution.

10.6 g of the tannic acid solution was added to the microcapsules suspension under mechanical agitation at 170 rpm at 50° C., and left stirring for 5.5 hours. After this time, the suspension was stored in the fridge for around 36 hours. The crosslinked microcapsule suspension was poured over a 38 μm sieve, washed with hard water pH3 and resuspended in a clean separating funnel in hard water pH3. The decanted capsules were collected a second time on the 38 μm sieve, washed with single strength buffer solution with preservative and resuspended in this buffer solution in the separating funnel. The capsules were collected as concentrated a slurry after decantation and appeared as brown coloured microencapsulates, indicating that crosslinking had occurred.

The crosslinked capsule slurry was directly strained through a 40 μm cell strainer for 5 minutes and any extra moisture was absorbed with a paper tissue. The total fragrance level was analysed by Gas chromatography by the method herein to be 4.1 wt %. This is lower than the total fragrance level of the microcapsules prior to cross-linking. Without wishing to be bound by theory it is believed that this is due to surface and loosely associated fragrance being removed by the washing process during the cross-linking. The particle size was measured by laser diffraction to give a D₅₀ of 47.9 μm. This is larger than the particle size of the original spray dried encapsulate indicating that the hydrogel shell has been hydrated and the encapsulate has swollen.

### Olfactive Evaluation of Spray Dried and Cross-Linked Microcapsules

A diluted slurry of the microcapsules with gellan gum was prepared so that it contained 0.1 wt % total fragrance. The gellan gum helped maintain the microcapsules well suspended. This was done according to the method in Example 18. The pH of the diluted slurry was 3.04. The odour of the diluted slurry was evaluated according to the method herein described, and the results are shown in Table 19.

TABLE 19

| Substrate | | Glass | Card |
|---|---|---|---|
| Initial | Strength | 2.8 | 3.1 |
| application | Character | Fruity, lemon, sweet, floral | Fruity, lemon, sweet, |
| | | | medicinal, tangerine peel |
| 24 hours | Strength | 1.1 | 0.7 |
| | Character | Sweet, fruity, floral | Floral |
| Rubbing | Strength | 4.0 | 3.9 |
| | Character | Sweet, ester, artificial fruit, | Sweet, fruity, ester, tangerine |
| | | tangerine peel, floral | peel, medicinal |
| Pressure | Strength | 4.25 | Not applicable |
| with a | Character | Fresh, citrus, tangerine peel, | Not applicable |
| slide | | fruity | |

On initial application there was a moderate characteristic odour for the fragrance indicating that a small amount of the fragrance is associated with the surface of the hydrogel shell. After 24 hours of drying the odour was very weak indicating that most of the surface fragrance had evaporated. On rubbing or pressing on glass a strong characteristic odour was noticed as the force ruptures that microcapsule releasing the fragrance. On rubbing on card a moderate odour was noticed and the fragrance character was similar to that on glass.

### Example 20—Preparation of Spray-Dried Microcapsules Containing Flaxseed Oil

### Preparation and Application of Shear to the Protein Hydrogel

The protein hydrogel slurry was prepared as described in Example 13. The protein solid content was measured as 9.5 wt %.

### Preparation of Spray-Dried Microcapsule

Flaxseed oil is a dietary supplement used for its high level of omega-3 fatty acids that is also a vegan alternative to fish oils. The slurry was homogenised with a Silverson L5M-A high-shear mixer for 2 minutes at 8000 rpm. Flaxseed oil was added to the slurry at a 1:1 oil:protein solids weight ratio and the mixture was homogenised further with the Silverson at 8000 RPM for 5 minutes to create a fine emulsion.

The slurry was spray-dried within a day of making, using a Buchi B290 Spray Dryer using a two-fluid nozzle with a tip size of 1.4 mm. The aspirator flow rate was set at 100%, with a Q-flow setting of 40. The fluid was pumped into the spray dryer with a peristaltic pump at a speed setting of 13% (4-5 ml/min). The air inlet temperature varied between 183 and 180° C. and the air outlet temperature between 126 and 122° C.

Dry microcapsule powder formed indicating that the oil was efficiently encapsulated. The particle size was measured via laser diffraction according the method herein and found to have a d50 of 45.3 microns. The powder had no rancid off-odour indicating that the temperature in the drier had not degrade the flaxseed oil. It had a weak nutty and very slight vinegar odour indicating that there was only a low level of surface oil.

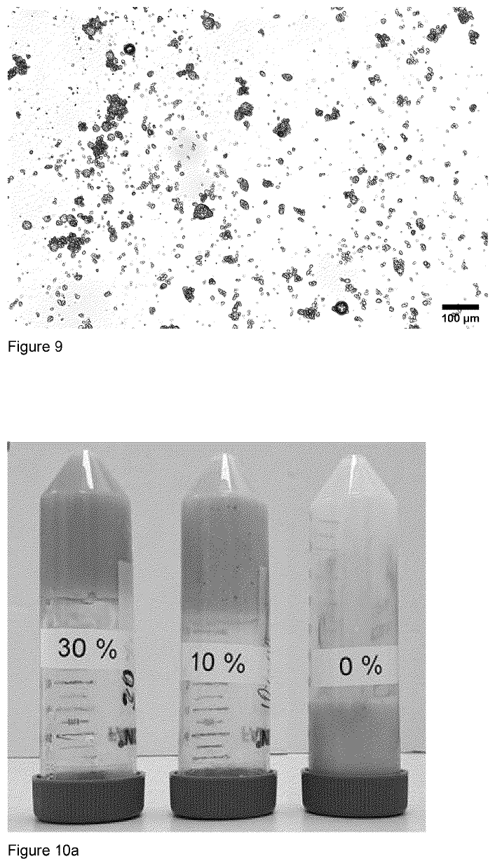

FIG. 9 shows a 10× optical microscope image of Example 20 suspended in single strength citrate buffer where it can be seen that the encapsulates remain intact.

The microcapsule powder of Example 20 was also suspended in Miwadi Blackcurrant single concentrate by vortexing at 2000 rpm for 1 minute at a level of 12.5 g/L. The encapsulate did not impart any negative odour to the characteristic red fruit note of the original product demonstrating that the encapsulate was insoluble and the flaxseed oil remained encapsulated thereby having very little impact on product aroma. In comparison the same level of flaxseed oil could not be incorporated into the beverage without emulsifiers and would have provided a very noticeable nutty odour and characteristic effect on the beverage taste.

### Example 21—Demonstration of Formation of Protein Gel

Reverse osmosis (RO) water and pea protein isolate was added to a 1-litre stainless steel container and placed in a 92° C. water bath and mixed with an overhead stirrer at 1300 rpm. After stirring for 3 minutes, glacial acetic acid was added. The mixture was stirred for a total of 45 minutes at 1300 rpm, ensuring that the temperature was maintained above 80° C. The mixture was then subjected to high shear with a Silverson L5M-A homogenizer, at 8000 rpm for 10 minutes.

20 ml aliquots of the hot liquid were measured into a 50 ml Falcon tubes, sealed and refrigerated overnight in a normal upright position. High levels of organic acid were tested as given in Table 20a.

5 g aliquots of the hot liquid were weighed into 14 ml glass vials, sealed and refrigerated overnight in a normal upright position. Lower levels of organic acid were tested as given in Table 20b.

TABLE 20a

| Example | 21A | 21B | 21C |
|---|---|---|---|
| Mass glacial acetic acid (g) | 0 | 50 | 150 |
| Mass pea protein isolate (g) | 50 | 50 | 50 |
| Mass water (g) | 450 | 400 | 300 |
| wt % Acetic acid | 0 | 10 | 30 |
| Weight ratio acetic acid:water | 0 | 1:8 | 1:2 |
| Weight ratio acetic acid:Protein solids | 0 | 1:1 | 3:1 |

TABLE 20b

| Example | 21D | 21E | 21F | 21G | 21H |
|---|---|---|---|---|---|
| Mass glacial acetic acid (g) | 0 | 5 | 10 | 15 | 25 |
| Mass pea protein isolate (g) | 50 | 50 | 50 | 50 | 50 |
| Mass water (g) | 450 | 445 | 440 | 435 | 425 |
| wt % Acetic acid | 0 | 1 | 2 | 3 | 5 |
| Weight ratio acetic acid:water | 0 | 1:445 | 1:220 | 1:145 | 1:85 |
| Weight ratio acetic acid:Protein solids | 0 | 1:10 | 1:5 | 3:10 | 1:2 |
| pH of mixture after homogenization | 6.21 | 4.16 | 3.87 | 3.66 | 3.50 |

After a minimum of 12 hours of refrigeration the formation of a gel was evaluated by inverting the vials.

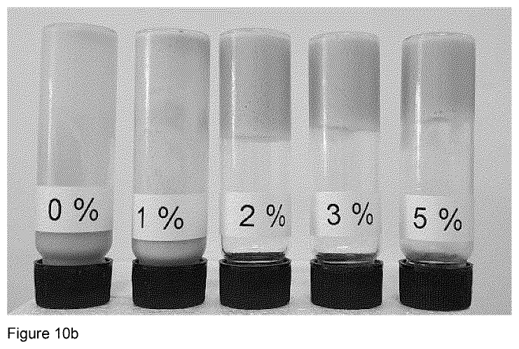

As can be seen in FIG. 10a, use of high level of acid, 30% and 10% glacial acetic acid, resulted in the formation of a solid gel that did not drop under gravity. Use of no acid did not form a gel and fell into the lid of the inverted Falcon tube.

As can be seen in FIG. 10b, use of 5%, 3% and 2% glacial acetic acid resulted in the formation of a solid gel that did not drop under gravity (within a 20 minute observation period). Use of 1% glacial acetic acid and no acid did not form a gel and fell into the lid of the inverted vial.

Relating the gel test results to pH measurements of the hot slurry after homogenization, the mixtures that formed gels were more than 0.5 units below the isoelectric point of the pea protein isolate, which is 4.5 according to Guldekin et al., Food Hydrocolloids (2023), 145:109029.

### Example 22—Preparation of Protein Slurries

A: Preparation of Protein Slurry with Acetic Acid and Sonication

336 g of Pea Protein Isolate (PPI) was added to 2731 g of room temperature reverse osmosis water and placed in a water bath at 90° C. The mixture was stirred with an overhead stirrer with a propeller impeller at 1700 rpm for around 1 to 2 mins to ensure homogeneous wetting of the PPI powder. 269 g of 80% (v/v) acetic acid solution was added to the stirred mixture, a lid placed over the vessel and stirring continued for another 45 mins. The temperature of the mixture was checked. If this was less than 85° C. stirring was continued until this temperature was reached.

The hot mixture was then transferred to a Silverson mixer and homogenised for 15 mins at 8000 rpm. The vessel was then covered and left to cool until the mixture temperature was below 40° C. which may take several hours, typically overnight.

Once this temperature was reached the mixture was gently stirred with an overhead mixer with an anchor impeller at 200 rpm for 15 mins to make the mixture pourable and it was divided into smaller 1 litre batches in bottles.

Each bottle containing around 860-890 g of mixture was sonicated using a Hielscher UIP500hdT whilst in an ice bath to ensure that the mixture temperature remains below 40C. This was done at 100% amplitude until the total energy applied was 175 kJ, over a period of 22-25 minutes. The sonication was stopped a quarter and half-way through the process to manually shake the bottle for a minute to ensure a homogeneous mixture. The mixture was then passed through a 200 micron sieve to produce a homogeneous low viscosity liquid. The viscosity was measured to be 81.8 mPas at 50 s⁻¹ and the pH was 3.5.

B: Preparation of Protein Slurry with Acetic Acid and High Pressure Homogenisation

The method for Example 22A was followed until the mixture was cooled below 40° C. The sample was processed using a Pressure Cell Homogeniser SPCH-EP Model FPG12805 (Homogenising System Ltd) equipped with a piston gap valve with a 1 mm contact diameter (HPVS-1). The slurry was put through 2 passes at 100 MPa. 0.1 wt % Methyl paraben preservative was added. The viscosity was measured to be 390.8 mPas at 50 s⁻¹ and the pH was 3.6.

C: Comparative Example: Preparation of Protein Slurry with No Acid

The method for Example 22B was followed with the changes being that in the formulation 2744.0 g reverse osmosis water was used and instead of acetic acid, 256 g of reverse osmosis water was added. The viscosity was measured to be 188.3 mPas at 50 s⁻¹ and the pH was 6.5.

D: Comparative Example: Preparation of Protein Slurry with HCl

The method for Example 22B was followed with the changes being that in the formulation 2658.7 g reverse osmosis water was used and instead of acetic acid, 341.3 g of 1M HCl was added. The viscosity was measured to be 204.8 mPas at 50 s⁻¹ and the pH was 3.1.

### Example 23—Preparation of Spray-Dried Microcapsules Containing Vitamin D2 with Modified Slurries

### Preparation and Application of Shear to the Protein Slurry

Protein slurries were prepared as described in Examples 22B and Comparative Examples 22C and 22D. The protein solid contents were all calculated at 10.07% wt.

### Preparation of Spray-Dried Microcapsule

Vitamin D2 oil (1 MIU/g) was diluted in the carrier Softisan® 100 wax to a concentration of 6000 ug/g. The wax was melted above 40° C., the Vitamin D2 oil added, and the mixture homogenised by hand shaking. The mixture was held above 40° C. until required.

The required mass of Vitamin D2 and wax was then added to each protein slurry 22B, C and D at a protein: carrier solids weight ratio 5:1. This was then mixed with a Silverson L5M-A homogenizer at 8000 rpm for 10 minutes to form emulsions of Mixtures 23 B, C and D with 11.5 weight % total solids including vitamin D2 and wax.

Each emulsion was spray-dried within a day of making, using a Buchi B290 Spray Dryer using a two-fluid nozzle with a tip size of 1.4 mm. The aspirator flow rate was set at 100%, with a Q-flow setting of 40. The air inlet temperature was kept at 180° C. The fluid was pumped into the spray dryer with a peristaltic pump at a speed setting of 6.7-23 g/min to achieve an outlet air outlet temperature between 85 and 120° C. The process conditions and results are given in Table 21.

TABLE 21

| | Encapsulate | | |
|---|---|---|---|
| | | Comparative | Comparative |
| | Example 23B | Example 23C | Example 23D |
| | Slurry | | |
| | | Comparative | Comparative |
| | Example 22B | Example 22C | Example 22D |
| Protein:carrier wt ratio | 5:1 | 5:1 | 5:1 |
| Carrier | Wax | Wax | Wax |
| Weight % total solids in | 11.5 | 11.5 | 11.5 |
| emulsion (including vitamin D2 | | | |
| and carrier) | | | |
| Emulsion Temperature (° C.) | 33.0 | 44.7 | 39.0 |
| Average Inlet Temperature (° C.) | 180 | 180 | 180 |
| Average Outlet Temperature | 98 | 116 | 109 |
| (° C.) | | | |
| Run Rate (g/mins) | 23 | 6.7 | 8.1 |
| Moisture Content (%) | 8.0 | 6.0 | 5.9 |
| Particle size (D50, μm) | 16.6 | 13.0 | 22.8 |
| Vitamin D2 (μg/g) | 1029.71 | 1018.95 | 1002.84 |

Dry microcapsule powder formed indicating that the wax and Vitamin D was encapsulated. The moisture content, particle size via laser diffraction and Vitamin D2 content (method Version B) and were all measured using the methods herein described.

All of the three Examples had very similar Vitamin D2 content. The large difference in run rates and outlet temperatures between Examples 23B and 23 C and 23D was due to the difference in viscosity, as the emulsion sample without added organic acid had lower viscosity than the emulsion sample made using organic acid. This is driven by the fact that they had been prepared with protein slurries with very different viscosities.

### Example 24-Preparation of Spray-Dried Microcapsules Containing Vitamin D2 with Different Carriers

### Preparation and Application of Shear to the Protein Slurry

A protein hydrogel slurry was prepared as described in Example 22A. The protein solid content was measured as 11.6 wt %.

### Preparation of Spray-Dried Microcapsule

Vitamin D2 oil (1 MIU/g) was diluted in the carrier Softisan® 100 wax to a concentration of 6000 ug/g. The wax was melted above 40° C., the Vitamin D2 oil added, and the mixture homogenised by hand shaking. The mixture was held above 40° C. until required.

Separately Vitamin D2 oil (1 MIU/g) was also separately diluted in the carrier olive oil to a concentration of 6000 ug/g and the mixture homogenised by hand shaking. The mixture was held at room temperature until required.

The required mass of Vitamin D2 and carrier was then added to the protein hydrogel slurry of Example 22A at a protein: carrier solids weight ratio of 5:1 and 2:1. This was then mixed with a Silverson L5M-A homogenizer at 7000 rpm for 10 minutes to form an emulsion.

The slurry was spray-dried using a pilot scale co-current spray dryer using a rotary disc atomizer. The drying chamber was approximately 1 m in diameter and 2.1 m in height. The nominal evaporative capacity was 3 kg H₂O/hr at 150° C., with a nominal airflow of 125 m³/hr. The fluid was pumped into the spray dryer using a peristaltic pump with the flow rate adjusted to achieve the target outlet temperature. The air inlet temperature varied between 179 and 181° C. and the air outlet temperature between 92 and 95° C. Processing conditions and results are given in Table 22.

TABLE 22

| Encapsulate | Example 24A | Example 24B | Example 24C |
|---|---|---|---|
| Protein:carrier wt ratio | 5:1 | 2:1 | 2:1 |
| Carrier | Wax | Wax | Olive oil |
| Weight % total solids in emulsion | 13.7 | 16.7 | 16.7 |
| (including vitamin D2 and carrier) | | | |
| Emulsion Temperature (° C.) | 20.3 | 41.5 | 16.5 |
| Rotary Disc Pressure | 30 | 50 | 50 |
| Run Rate (g/mins) | 60 | 55 | 49 |
| Moisture Content (%) | 7.7 | 7.1 | 4.8 |
| Particle size (D50, μm) | 56 | 45 | 51 |
| Vitamin D2 (μg/g) | 1062 | 1322 | 1369 |

Dry microcapsule powder formed indicating that the Vitamin D2 and carrier was encapsulated. The moisture content, particle size via laser diffraction and Vitamin D2 content (method Version B) and were all measured using the methods herein described.

The Vitamin D2 content of the microcapsule powders was in all cases higher than the target of 1000 ug/g. In Examples 24B and C, the increased level may be due to uneven distribution of oil and protein between the sample obtained from the collection vessel, and smaller particles not recovered by the cyclone.

### Example 25—Preparation of Spray-Dried Microcapsules Containing Vitamin D2 & Core Additives

### Preparation and Application of Shear to the Protein Hydrogel

A protein hydrogel slurry was prepared as described in Example 22A. The protein solid content was measured as 11.8 wt %.

A protein hydrogel slurry was also prepared as described in Example 13. The protein solid content was measured as 10.2 wt %.

### Preparation of Spray-Dried Microcapsule

Vitamin D2 oil (1 MIU/g) was diluted in Softisan® 100 wax to a concentration of 6000 ug/g. The wax was melted above 40° C., the Vitamin D2 oil added, and the mixture homogenised by hand shaking. A solution of 10 mg/g of curcumin in absolute ethanol was added to the mixture, with the curcumin at 0.46% wt of the mass of Vitamin D2 in the emulsion. This Mixture 25A was homogenised by hand shaking and held above 40° C. until required.

Vitamin D2 oil (1 MIU/g) was diluted in Softisan® 100 wax to a concentration of 6000 ug/g. The wax was melted above 40° C., the Vitamin D2 oil added, and the mixture homogenised by hand shaking. A solution of 10 mg/g curcumin in absolute ethanol was added to the mixture, with the curcumin at 0.62% wt of the mass of Vitamin D2 in the emulsion. This Mixture 25B was homogenised by hand shaking and held above 40° C. until required.

Vitamin D2 oil (1 MIU/g) was diluted in Softisan® 100 wax to a concentration of 6000 ug/g. The wax was melted above 40° C., the Vitamin D2 oil added, followed by Vitamin E at 10% wt of the mass of Vitamin D2 in the emulsion. This Mixture 25C was homogenised by hand shaking and held above 40° C. until required.

Vitamin D2 oil (1 MIU/g) was diluted in Softisan® 100 wax to a concentration of 6000 ug/g.

The wax was melted above 40° C., the Vitamin D2 oil added, and the mixture homogenised by hand shaking. A solution of 100 mg/g beta-carotene in absolute ethanol was added to the mixture, with the beta-carotene at 6.2% wt of the mass of Vitamin D2 in the emulsion. This Mixture 25D was homogenised by hand shaking and held above 40° C. until required.

The required mass of Mixtures 25 A, C and D were each then added to the protein hydrogel slurry of Example 22A and Mixture 25B was added to the protein hydrogel slurry of Example 13. The wax was added to the slurry at a protein: wax solids weight ratio of 5:1. This was then mixed with a Silverson L5M-A homogenizer at 7000 rpm for 10 minutes to form an emulsion with 13.8 weight % total solids including vitamin D2, wax and additive for the emulsions of Mixtures 25 A, C and D and 11.9 weight % total solids including vitamin D2, wax and additive for the emulsion of Mixture 25B.

The slurry was spray-dried within a day of making, using a Buchi B290 Spray Dryer using a two-fluid nozzle with a tip size of 1.4 mm. The slurry was first heated to between 40° C. and 45° C. The aspirator flow rate was set at 100%, with a Q-flow setting of 40. The fluid was pumped into the spray dryer with a peristaltic pump at a speed setting of 35% (13-14 ml/min). The air inlet temperature varied between 181 and 179° C. and the air outlet temperature between 127 and 111° C. Processing conditions and results are given in Table 23.

TABLE 23

| | Encapsulate | | | |
|---|---|---|---|---|
| | Example | Example | Example | Example |
| | 25A | 25B | 25C | 25D |
| | Slurry | | | |
| | Example | Example | Example | Example |
| | 22A | 13 | 22A | 22A |
| | Core Additive | | | |
| | | | Vitamin | Beta- |
| | Curcumin | Curcumin | E | carotene |
| Weight % total solids in emulsion | 13.8 | 11.9 | 13.8 | 13.8 |
| (including vitamin D2 and carrier) | | | | |
| Run Rate (g/mins) | 5.3 | 6.8 | 4.0 | 3.5 |
| Moisture Content (%) | 8.0 | 8.0 | 6.0 | 6.0 |
| Particle size (D50, μm) | 17 | 24 | 18 | 17 |
| Vitamin D2 (μg/g) | 1081.32 | 1152.39 | 1147.12 | 783.97 |

Dry microcapsule powder formed indicating that the Vitamin D and carrier was encapsulated. It was noted that the additives slightly modified the overall creamy colour of the powder. Examples 25A and B were pale yellow, Example 25C was whiter and Example 25D was pale orange. The moisture content, particle size via laser diffraction and Vitamin D2 content (method Version B) and were all measured using the methods herein described.

### Example 26—Integrity of Spray-Dried Microcapsules Containing Vitamin D2

Integrity of the microcapsules was determined according to the method herein described and results are given in Table 24. For each encapsulate 1% of dry powder was added to the water to ensure that the Vitamin D level was within the method detection limit.

A comparative test was performed using a commercially available polysaccharide Vitamin D microcapsule, Prinova GA. The Vitamin D content in the powder was measured as 2727.4 ug/g using the Method B herein described and the moisture as 7.4 wt % using the method herein described.

TABLE 24

| | | Vitamin D2 in | Vitamin D2 |
|---|---|---|---|
| Microcapsule Example | Protein treatment | supernatant (μg/g) | leakage (%) |
| Comparative Prinova Gum | Polysaccharide | 20.49 | 75.2 |
| Acacia Microcapsule | microcapsule | | |
| Example 23B | Acetic acid, wax | 0 (below detection | 0 |
| | carrier | limit, <<0.5 ug/g) | |
| Example 24C | Acetic acid, olive oil | 0 (below detection | 0 |
| | carrier | limit, <<0.5 ug/g) | |
| Comparative Example 23C | No Acid, wax | 1.72 | 15.3 |
| | carrier | | |
| Comparative Example 23D | HCl, wax carrier | 0.81 | 7.8 |

The Comparative Prinova GA microcapsules are highly soluble in water so they do not form a visible sediment. As they are highly soluble they release most of the encapsulated Vitamin D2 into the water. Leakage was measured at 75%.

The inventive compositions of Example 23B and Example 24C on the other hand are highly insoluble in water and maintained their integrity. 0% of the encapsulated Vitamin D2 leaked out.

Comparative Example 23C and 23D had 15 and 8% leakage demonstrating the importance of treating the plant protein with an organic acid.

TABLE 25

| Microcapsule | Active | Vitamin D2 in | Vitamin D2 |
|---|---|---|---|
| Example | protectant | supernatant (μg/g) | leakage (%) |
| Example 25A | Curcumin | 0 (below detection limit) | 0 |
| Example 25B | Curcumin | 0 (below detection limit) | 0 |
| Example 25C | Vitamin E | 0 (below detection limit) | 0 |
| Example 25D | Beta-Carotene | 0 (below detection limit) | 0 |

The inventive compositions of Examples 25A, B, C and D maintained their integrity and had 0% leakage demonstrating that using an active protectant does not adversely affect the microcapsules' integrity as shown in Table 25.

### Example 27—Accelerated Stability Testing of Vitamin D2 Microcapsules in a Beverage Formulation

### Preparation of Beverage Samples

The microcapsules of Examples 24A and B and Examples 25 A, B, C, D were each prepared into a concentrated slurry so that they could then be added to MiWadi Blackcurrant single concentrate squash.

0.2 g of each microcapsule was first suspended in 30 g of boiling water with vigorous shaking, followed by vortexing at 2000 rpm for 1 minute to mimic the temperature profile during flash pasteurization.

### Accelerated Stability Testing

A calculated mass of each concentrated slurry was added to 2 liters of squash and mixed with an overhead mixer at 500 rpm for 5 minutes to ensure homogeneity before transferring to bottles.

200 ml transparent plastic PET bottles were filled with each of the three squash samples and initial Vitamin D2 loading was measured using Method B herein. The filled PET bottles were then placed in a Binder KBF LQC 720 chamber, set to run with only the visible light bulbs. The total light doses for the study were determined by the expected visible light intensity in a typical supermarket (commonly 600 lux), running 24 hours per day for 6 months or 9 months. This corresponded to 2.6 MLUXh (representing 6 months on shelf) or 3.9 MLUXh (representing 9 months on shelf) at 35° C. and laboratory humidity (typically 50% relative humidity (RH)). The light photometry function on the Binder KBF LQC 720 was used to achieve the required dose. The Vitamin D loading was again measured, using the method B herein described, after the equivalent of 6 and 9 months on the shelf. The % reduction in Vitamin D2 content was calculated and recorded in Table 26 for Examples 24 A and, B, and in Table 27 for Examples 25 A, B, C, D.

TABLE 26

| | | | Moisture | % Vitamin | % Vitamin D2 |
|---|---|---|---|---|---|
| Microcapsules | Ratio | D50 | content | D2 loss after | loss after 3.9 |
| Example | protein:wax | (μm) | (% wt) | 2.6 MLUXh | MLUXh |
| Example 24A | 5:1 | 56 | 7.7 | 33 | 48 |
| Example 24B | 2:1 | 45 | 7.1 | 26 | 46 |

The results in Table 26, show that after the equivalent of 9 months shelf life in the drink, the Vitamin D2 in the encapsulate of the invention had been protected from degradation. Unencapsulated Vitamin D2 is expected to degrade to below the method detection limit on exposure to light over this time frame. For Examples 24A and 24B with a ratio of protein to wax greater than 1:1 there was a loss of only 48% and 46% respectively of the initial Vitamin D2. This low level of loss demonstrates the protection provided by the inventive microcapsule during long-term storage.

TABLE 27

| | | | Moisture | % Vitamin | % Vitamin D2 |
|---|---|---|---|---|---|
| Microcapsule | Active | D50 | content | D2 loss after | loss after 3.9 |
| Example | protectant | (μm) | (% wt) | 2.6 MLUXh | MLUXh |
| Example 25A | Curcumin | 17 | 8.0 | 44 | 54 |
| Example 25B | Curcumin | 24 | 8.0 | 30 | 41 |
| Example 25C | Vitamin E | 18 | 6.0 | 36 | 48 |
| Example 25D | Beta-Carotene | 17 | 6.0 | 38 | 47 |

The results in Table 27, show that after the equivalent of 9 months shelf life in the drink, the Vitamin D2 in the encapsulate of the invention had been protected from degradation in spite of the very small average particle size, resulting from the spray dryer used. Unencapsulated Vitamin D2 is expected to degrade to below the method detection limit on exposure to light over this time frame. Example 25B had the lowest loss of Vitamin D2 at 41% which, without wishing to be bound by theory, is believed to be due to the combination of the protective core additive of curcumin and the larger average particle size. Examples 25 A, C and D all had slightly higher loss than Example 25B which is believed to be due to the very small average particle size. Nonetheless the core additives provided additional protection so that their stability performance was in line with Example 24A which has a much larger average particle size. This low level of loss demonstrates the protection provided by the inventive microcapsule during long-term storage. Without wishing to be bound by theory, it is believed that the larger the average particle size of the encapsulate, the lower the surface area available per mass of Vitamin D2 for exposure to light and therefore the less the degradation.

### Example 28—Preparation of Spray-Dried Microcapsules Containing Fragrance with Shell Additives

### Fragrance/Solvent Mixtures

Fragrance 28A was prepared according to Table 28 and blended with Miglyol® 812N in a weight ratio of 80:20 fragrance: solvent. Fragrance 28A comprises materials of similar hydrophobicity with the full range of volatilities including low, moderate and high volatility materials. The fragrance had fragrance materials with HSP parameters in the range δD from 15 to 20, δP from 1 to 7 and δH from 2.5 to 8.

TABLE 28

| | | Vapour | | | | | | |
|---|---|---|---|---|---|---|---|---|
| Fragrance | CAS | pressure | | HSP | | | | |
| material | number | (Torr) | LogP | δD | δP | δH | Functionality | wt % |
| Dipentene | 138-86-3 | 1.541 | 4.40 | 17.2 | 1.8 | 4.3 | Alkene | 20 |
| Ortho tertiary | 88-41-5 | 0.103 | 4.46 | 15.9 | 1.9 | 2.9 | Ester | 20 |
| butyl cyclo | | | | | | | | |
| hexyl acetate | | | | | | | | |
| (OTBCHA) | | | | | | | | |
| Diphenyl oxide | 101-84-8 | 0.022 | 4.03 | 19.4 | 3.4 | 4.0 | Ether | 20 |
| Delta | 57378-68-4 | 0.010 | 3.56 | 16.9 | 3.5 | 2.6 | Ketone | 20 |
| damascone | | | | | | | | |
| Peach | 95962-14-4 | 0.0004 | 4.20 | 17.7 | 4.2 | 3.3 | Ketone, | 20 |
| cyclopentanone | | | | | | | alkene | |

### Preparation of Spray Dried Microcapsules

A first batch of fragrance composition was prepared as follows. A batch of protein hydrogel slurry of Example 13 was homogenized with a Silverson lab mixer at 8000 rpm for 2 minutes. 5.1 wt % (based on the weight of the slurry) of Fragrance 28A/Miglyol® 812N blend was then added to the slurry and emulsified using an overhead stirrer with a 4-blade impeller at 800 rpm for 2 minutes to form a coarse emulsion. This emulsion was used in Example 28B.

A second batch of fragrance composition was prepared as follows. A batch of protein hydrogel slurry of Example 13 was combined with 2.0 wt % (based on the weight of the slurry) of glycerol. The mixture was homogenized with a Silverson lab mixer at 8000 rpm for 2 minutes. 6.1 wt % (based on the weight of the slurry) of Fragrance 28A/Miglyol® 812N blend was then added to the slurry and emulsified using an overhead stirrer with a 4-blade impeller at 800 rpm for 2 minutes to form a coarse emulsion. This emulsion was used in Example 28C.

A third batch of fragrance composition was prepared as follows. A batch of protein hydrogel slurry of Example 13 was homogenized with a Silverson lab mixer at 8000 rpm for 2 minutes. 4.9 wt % (based on the weight of the slurry) of Fragrance 28A/Miglyol® 812N blend was then added to the slurry and emulsified using a Silverson mixer at 8000 rpm for a further 5 minutes to form a fine emulsion. This mix was used in Example 28D.

A fourth batch of fragrance composition was prepared as follows. A batch of protein hydrogel slurry of Example 13 was heated to 70° C. in a water bath and combined with 3.6 wt % (based on the weight of the slurry) of Capsul® Starch. The mixture was homogenized with a Silverson lab mixer at 8000 rpm for 2 minutes. The mixture was cooled to ambient temperature overnight. 6.4 wt % (based on the weight of the slurry) of Fragrance 28A/Miglyol® 812N blend was then added to the slurry and emulsified using a Silverson mixer at 8000 rpm for a further 5 minutes to form a fine emulsion. This emulsion was used in Example 28E.

Each of the emulsions was spray-dried using a pilot scale co-current spray dryer using a rotary disc atomizer. The drying chamber was approximately 1.2 m in diameter and 2.1 m in height. The nominal evaporative capacity was 3 kg H₂O/hr at 150° C., with a nominal airflow of 125 m3/hr. The fluid was pumped into the spray dryer using a peristaltic pump with the flow rate adjusted to achieve the target outlet temperature. The air inlet temperature varied between 129 and 135° C. and the air outlet temperature between 69 and 70° C.

Fragrance 28A was successfully encapsulated in Examples 28B, C, D and E as shown in Table 29.

TABLE 29

| | Example 28B | Example 28C | Example 28D | Example 28E |
|---|---|---|---|---|
| Shell | Plant | Plant Protein + | Plant | Plant Protein + |
| | Protein | Glycerol | Protein | Capsul ® Starch |
| Emulsion type | Coarse | Coarse | Fine | Fine |
| Feed rate | 53 | 42 | 47 | 45 |
| (g/min) | | | | |
| Particle size | 33 | 37 | 57 | 40 |
| (D50, μm) | | | | |
| Total Fragrance | 18.7 | 16.2 | 20.3 | 18.0 |
| (% weight) | | | | |

In all 4 examples dry microcapsule powder formed indicating that the fragrance was encapsulated. The particle size via laser diffraction and fragrance content were measured using the methods herein described. The addition of glycerol or Capsul® Starch to the encapsulate shell did not have much an effect on the fragrance loading or particle size. The use of a fine emulsion resulted in larger average encapsulate particle size.

The individual fragrance materials in oil Example 28A were measured using the method herein as shown in Table 30.

TABLE 30

| | Example 28B | Example 28C | Example 28D | Example 28E |
|---|---|---|---|---|
| Fragrance material | Fragrance % | Fragrance % | Fragrance % | Fragrance % |
| | weight | weight | weight | weight |
| Dipentene | 14.4 | 14.2 | 15.0 | 14.4 |
| Ortho tertiary butyl cyclo | 21.9 | 23.2 | 21.3 | 22.2 |
| hexyl acetate (OTBCHA) | | | | |
| Diphenyl oxide | 22.5 | 20.7 | 19.6 | 22.8 |
| Delta damascone | 13.9 | 11.4 | 14.3 | 15.6 |
| Peach cyclopentanone | 27.3 | 30.6 | 29.8 | 25.0 |

All of the fragrance components were encapsulated however not quite equally at 20 wt % each indicating that there is allow level of selectivity. There is very little difference between the 4 encapsulates indicating that the shell modifications do not affect any fragrance selectivity during encapsulation.

Delta-damascone and dipentene had the lowest encapsulation levels. Delta-damascone is the fragrance material with the lowest log P. Without wishing to be bound by theory it is expected higher log P materials will be more easily emulsified and therefore preferentially encapsulated. Dipentene is the most volatile material and so is most likely to be lost by evaporation in the drying process. However, it was still present at about three quarters of the level expected.

The encapsulates were also assessed for their olfactive performance according to the olfactive evaluation method herein. Example 28C performed as well as Example 28B with intensity grades of 3.5 when rubbing and pressing on glass, and 4.5 when rubbing on blotter. Example 28E performed as well as Example 28D with intensity grades of 3.5 when rubbing on glass and 4.0 when pressing on glass and rubbing on blotter. This confirms that the addition of Glycerol or Capsul® Starch does not prevent the dry encapsulate from breaking when rubbed or pressed and so releasing the fragrance so it can be smelled.

1. A method for the preparation of a biodegradable microcapsule, the method comprising:
(a) forming a mixture comprising one or more plant-based protein(s) in a solvent system, wherein the solvent system comprises miscible co-solvents; wherein a first co-solvent increases solubility of the plant-based protein(s), and a second co-solvent decreases solubility of the plant-based protein(s); wherein the co-solvents are added to the mixture either in concentrated or in a diluted form; and wherein the pH of the plant-based protein mixture is at least 0.5 pH units below the isoelectric point of the plant-based protein;
(b) subjecting the plant-based protein mixture to a shear treatment to form a plant-based protein hydrogel slurry;
(c) dispersing an active ingredient(s) in said plant-based hydrogel slurry to form a composition; and
(d) drying said composition to form a microcapsule.

2. The method according to claim 1, further comprising the step of inducing at least part of the plant-based protein in the mixture to form a plant-based protein hydrogel by one or more of cooling, addition of salt, addition of anti-solvent, solvent reduction, addition of cross-linking agent, or electrostatic cross-linking.

3. The method according to claim 2, wherein inducing at least part of the plant-based protein in the mixture to form a plant-based protein hydrogel involves a sol-gel-transition.

4. The method according to claim 1, wherein the mixture formed in step (a) is a slurry, a dispersion, an emulsion, or a solution.

5. The method according to claim 1, wherein drying in step (d) is done by spray drying.

6. The method according to claim 1, wherein drying in step (d) is done by fluid bed drying.

7. The method according to claim 1, wherein the plant-based protein(s) is selected from pea protein, potato protein, rapeseed protein, lentil protein, chickpea protein, fava bean protein, mung bean protein, sunflower seed protein, pumpkin seed protein, flax protein, chia protein, canola protein, lupine protein, alfalfa protein, moringa protein and/or rice protein.

8. The method according to claim 1, wherein the first co-solvent is an organic acid selected from the group consisting of acetic acid, lactic acid, formic acid, propionic acid, an α-hydroxy acid, and/or a β-hydroxy acid.

9. The method according to claim 1, wherein the second co-solvent is selected from water, ethanol, and/or ethyl acetate.

10. The method according to claim 3, wherein the sol-gel transition is achieved by heating the protein mixture to a first temperature above the sol-gel transition temperature of the one or more plant-based protein(s) mixture, then reducing to a second temperature below the sol-gel transition temperature of the one or more plant-based protein(s) mixture to form a hydrogel.

11. A method according to claim 1, wherein said shear treatment comprises a single shear step that involves fragmenting the plant-based protein hydrogel into fragments wherein said fragments have a d₅₀ as determined by laser diffraction of 0.2 to 50 microns.

12. A method according to claim 1, wherein said shear treatment comprises a first shear step followed by a second shear step.

13. A method according to claim 12, wherein said first shear step involves fragmenting the plant-based protein hydrogel into fragments wherein at least 80 wt % of said fragments produced in said first shear step have a particle dimension in the range 1 mm to 100 mm as determined by optical microscopy.

14. A method according to claim 12, wherein said second shear step involves further fragmenting the plant-based protein hydrogel wherein said fragments produced in said second shear step have a d₅₀ as determined by laser diffraction of 0.2 to 50 microns.

15. A method according to claim 12, wherein step (b) further comprises subjecting the plant-based protein hydrogel slurry to a solvent reduction step between said first shear step and said second shear step.

16. A method according to claim 15, wherein said solvent reduction step comprises the steps of:
(i) contacting the fragments of the plant-based hydrogel slurry with a non-solubilising solvent;
(ii) separating the fragments of the plant-based hydrogel slurry from the non-solubilising solvent to give a washed plant-based protein hydrogel slurry; and
(iii) optionally repeating steps (i) and (ii).

17. A method according to claim 1, further comprising a step of altering the pH of the plant-based protein hydrogel slurry such that it is different to the isoelectric point of the plant-based protein by more than 1 pH unit.

18. A method according to claim 17, wherein said step of altering the pH of the plant-based protein hydrogel slurry is carried out after step (b) or sequentially with step (b).

19. A method according to claim 1, wherein the composition formed in step (c) is a shear thinning composition.

20. A method according to claim 1, wherein the composition formed in step (c) has a viscosity in the range 1 to 10000 cP at 20° C. and 50 s⁻¹.

21. A method according to claim 1, wherein the composition formed in step (c) has a protein solids content in the range 1 wt % to 25 wt % based upon the total weight of the composition.

22. A method according to claim 1, wherein said active ingredient(s) is selected from a vitamin, a mineral, a flavour material, a fragrance material, a pro-flavour, a pro-fragrance, a flavour enhancer, a malodour counteractant, a nutraceutical, a live organism (e.g. a probiotic), a pharmaceutical, an anti-microbial agent, an anti-viral agent, an anti-inflammatory agent, a pesticide, a herbicide, a fertiliser, a fungicide, an insecticide, an animal repellent, an anti-acne agent, a skin lightening agent, an emollient, a skin moisturizing agent, a wrinkle control agent, a fabric softener active, a surface cleaning active, a skin conditioning agent, a hair conditioning agent, a dye, a pigment, and an adhesive, or combinations thereof.

23. A method according to claim 22, wherein said active ingredient(s) is at least one fragrance material or flavour material and wherein:
the at least one fragrance material or flavour material has a vapour pressure greater than or equal to 0.00001 Torr at 25° C.; and/or
the at least one fragrance material or flavour material has a log P greater than or equal to 3.0.

24. A method according to claim 22, wherein said active ingredient(s) is at least one fragrance material or flavour material which is part of a fragrance or flavour wherein the fragrance or flavour contains at least 20 wt % of fragrance material(s) or flavour material(s) with a log P greater than or equal to 3.0 based upon the total weight of the fragrance or flavour.

25. A method according to claim 22, wherein said active ingredient(s) is a vitamin or a mineral selected from the group consisting of Vitamin A, Vitamin B1, Vitamin B2, Vitamin B3, Vitamin B5, Vitamin B6, Vitamin B7, Vitamin B9, Vitamin B12, Vitamin C, Vitamin D, Vitamin E, Vitamin K, magnesium, sodium, potassium, zinc, iron, calcium, iodine, omega 3, folic acid, thiamin, riboflavin, niacin and phosphorous, or mixtures thereof.

26. A method according to claim 1, wherein said active ingredient(s) is part of a composition comprising the active ingredient(s) and an active carrier phase.

27. A method according to claim 26, wherein said active carrier phase is a solvent, a fat or a wax.

28. A method according to claim 1, wherein the composition formed in step (c) comprises droplets of said active ingredient(s) or said composition comprising the active ingredient(s) and an active carrier phase having a dso as determined by laser diffraction of 0.2 to 50 microns.

29. A method according to claim 1, wherein plant-based protein residues formed during step (d) but not incorporated into the microcapsule are recycled in the method.

30. A method according to claim 1, further comprising subjecting the microcapsule to a post-treatment step, wherein the post-treatment step comprises a non-covalent cross-linking step, a covalent cross-linking step or a coating formation step, wherein the coating formation step comprises:
(i) treating the microcapsule with a metal or silicon-containing compound; and/or
(ii) subjecting the microcapsule to a complex coacervation step using a polysaccharide; and/or
(iii) treating the microcapsule with an aqueous mineral solution.

31. A biodegradable microcapsule obtained by or obtainable by the method of claim 1.

32-52. (canceled)
